(19) United States
(12) Patent Application Publication
(10) Pub. No.: US 2025/0302997 A1
(43) Pub. Date: Oct. 2, 2025

(54) IMPROVED VACCINIA VIRUS VECTORS

(71) Applicant: STRATOSVIR LIMITED, Hertfordshire (GB)

(72) Inventors: Christopher ULLMAN, Hertfordshire (GB); Juan Antonio POSTIGO, Hertfordshire (GB); Jason MERCER, Hertfordshire (GB)

(21) Appl. No.: 18/723,770

(22) PCT Filed: Dec. 23, 2022
(86) PCT No.: PCT/EP2022/087815

§ 371 Date: Jun. 24, 2024

(30) Foreign Application Priority Data
Dec. 24, 2021 (GB) 2119008.7

(51) Int. Cl. A61K 48/00; C12N 15/86

(52) U.S. Cl. CPC A61K 48/0058; C12N 15/86; C12N 2710/24122; C12N 2710/24143; C12N 2710/24162

(57) ABSTRACT

Disclosed herein are polynucleotides encoding fusion proteins, the fusion proteins comprising a vaccinia virus envelope protein, such as A13 or part thereof and at least one complement regulatory protein, such as CD35, CD55, CD59, CD46, CR1, Factor H, VCP, MOPICE, SPICE and CCPH, or a functional fragment thereof. Modified vaccinia virus vectors and vaccinia virus virions are also disclosed, as are therapeutic uses and methods for treatment of cancers and/or proliferative diseases or disorders.

### CROSS REFERENCE TO RELATED APPLICATIONS

This application is a § 371 U.S. National Stage Application of International Application No. PCT/EP2022/087815, filed Dec. 23, 2022, and claims priority to GB 2119008.7, filed Dec. 24, 2021, the entire contents of which are incorporated herein by reference.

### SEQUENCE LISTING

A sequence listing XML having file name Sequence_Listing_STRA0001 PA.XML (111,004 bytes) created on Jun. 24, 2024 is incorporated herein by reference in its entirety.

### FIELD OF THE INVENTION

The invention relates to the field of viral immunotherapy, e.g. for the treatment of cancers and/or cell proliferation diseases or disorders using viruses. In particular, the invention relates to engineered nucleic acids and genetically modified vaccinia viruses, as well as therapeutic uses and methods of treating cancers and/or proliferative diseases or disorders using the same.

### BACKGROUND OF THE INVENTION

Despite advances in new therapeutics, the survival rates for patients with many cancer types (particularly solid tumours) remain as one of the biggest challenges in medicine today.

Oncolytic viruses are attractive therapeutics for treatment of cancers, especially of those that may be resistant to current conventional therapies (Wong et al., (2010), Viruses 2, 78-106). Oncolytic viruses can selectively infect and replicate in tumour cells, causing cell death and stimulation of the immune system both through innate and adaptive immune responses. This is known as viral immunotherapy and is a rapidly growing area of cancer research. However, despite much effort to improve the selective activity of viruses against different tumour types, viral immunotherapy is limited because viruses are rapidly recognised and eliminated in vivo by the immune system before they can reach and infect target cells. New approaches that effectively address this problem are needed to expand the use of viral immunotherapy beyond tumours that can be directly injected, with a viral bolus, to those that are more visceral or disseminated.

Vaccinia virus has been used successfully to immunise against and, ultimately, eradicate smallpox. Furthermore, modified forms have shown promise in human clinical trials to treat cancer or to deliver antigens. Owing to the success of the vaccinia vaccines to eradicate smallpox from the human population, it is no longer necessary to immunise against the disease and, therefore, many people now lack immunity and antibodies to vaccinia virus. This makes vaccinia an interesting choice for immunotherapy, yet, despite the lack of neutralising antibodies on first administration, vaccinia is rapidly inactivated when administered intravenously, primarily because of the action of components of the innate immune system against the viral envelope.

Therefore, it would be desirable to have an improved system and viruses for a therapeutic treatment, such as for cancer therapy, e.g. by providing engineered vaccinia virus capable of prolonged exposure of viable virus within a host organism.

The present invention seeks to overcome or at least alleviate one or more of the problems found in the prior art.

### SUMMARY OF THE INVENTION

In general terms, the present invention provides new engineered vaccinia viruses and encoding nucleic acid molecules, compositions and related uses and methods that can be used for the treatment of diseases or pathogenic conditions in vitro and/or in vivo. In particular, by engineering complement control proteins as fusions with exposed viral envelope proteins, this invention provides engineered viruses capable of evading the immune response of an animal subject, e.g. when administered systemically in vivo. Accordingly, the invention provides vaccinia viruses with extended viability in vivo, such as in the bloodstream of a subject or patient.

In this way the engineered vaccinia viruses of the invention may survive long enough in vivo to sustain infection and spread to and within target distal tumour cells.

The viruses, compositions and methods of the present invention may be suitable for the treatment of any disease that may be treatable by providing an active agent—particularly a therapeutic vaccinia virus according to the invention—to target cells.

The compositions and methods of the present invention may be particularly useful in the treatment of cancers and/or proliferative diseases or disorders.

In one aspect there is provided an isolated nucleic acid encoding a fusion polypeptide, the fusion polypeptide comprising a vaccinia virus envelope protein or part thereof and at least one complement regulatory protein or functional fragment thereof. Suitably, a functional fragment of a complement regulatory protein is a fragment of a full length complement regulatory protein that is sufficient to cause directly or indirectly the destruction of a complement protein in a physiological system. In particular, the vaccinia virus envelope protein may be selected from A13 and A27 or part thereof. Most suitably the vaccinia virus envelope protein is A13.

In various embodiments, the at least one complement regulatory protein is selected from one or more of the group consisting of: (i) CD35, CD55, CD59, CD46, CR1, Factor H, VCP, MOPICE, SPICE, CCPH, C4-binding protein, CD35, Kaposi-sarcoma associated herpesvirus Kaposica/KCP, Herpesvirus saimiri (HVS) and HVS-CD59, Rhesus rhadinovirus RCP-H and RCP-1, murine gammaherpesvirus 68 (yHV-68) RCA, Influenzavirus M1, EMICE and IMP, as well asmodified sequences thereof, or functional fragments thereof; or (ii) CD35 CD55, VCP, mutated VCP, SPICE, CCPH and ORF4 or functional fragments thereof.

In embodiments the at least one complement regulatory protein or functional fragment thereof is fused to a vaccinia virus MV envelope protein transmembrane region.

In embodiments of this and any other aspect, the vaccinia virus envelope protein transmembrane region is not H3 or D8.

The vaccinia virus envelope protein A13 may be selected from an A13 protein sequence from Vaccinia Copenhagen virus, Camelpox virus, Variola virus, Cowpox virus, Taterapox virus, Monkeypox virus Zaire-96-I-16, Volepox virus, Akhmeta virus, Ectromelia virus, Orthopoxvirus Abatino virus, Skunkpox virus, Raccoonpox virus, Yokapox virus, Murmansk poxvirus, NY_014 poxvirus, and Yaba monkey tumor virus or part thereof. The vaccinia virus envelope protein A13 may comprise at least amino acids 2 to 21 of a sequence selected from one of SEQ ID NOs: 1 to 16, or a sequence having at least about 90%, at least about 95%, at least about 98% or at least about 99% sequence identity thereto.

Various embodiments of the disclosure provide a fusion polypeptide selected from: A13 fused to VCP, A13 fused to two VCP proteins arranged in tandem, A13 fused to four VCP proteins arranged in series, A13 fused to a mutant VCP, A13 fused to CD55, A13 fused to CD35, A13 fused to CCPH, or A13 fused to ORF4 or functional fragments thereof; optionally, wherein the mutant VCP comprises SEQ ID NO: 31; wherein CD55 comprises amino acids 35 to 284 of CD55 (e.g. SEQ ID NO: 71), wherein the CD35 comprises amino acids 42 to 1584 of CD35 (e.g. SEQ ID NO: 72), wherein the CCPH comprises amino acids 21 to 266 of CCPH (e.g. SEQ ID NO: 73) or wherein the ORF4 comprises amino acids 22 to 268 of ORF4 (e.g. SEQ ID NO: 74). In embodiments, the VCP protein is a poxvirus complement control protein or modified poxvirus complement control protein selected from SPICE (SEQ ID NO: 32), MOPICE (SEQ ID NO: 33), EMICE (SEQ ID NOs: 75, 76—particularly SEQ ID NO: 76) or IMP (SEQ ID NOs: 77, 78—particularly SEQ ID NO: 78).

In embodiments the one or more complement regulatory proteins or functional fragments thereof is fused to the C-terminus of A13 protein. In other embodiments, the one or more complement regulatory proteins or functional fragments thereof is fused to the N-terminus of A27 protein.

The disclosure encompasses nucleic acid molecules that encode polypeptides that are similar according to function and/or sequence (but not identical) to those disclosed herein. Thus, encompassed are polynucleotides having the sequence of any one of SEQ ID NOs: 37 to 46 or 57 to 62, or a polynucleotide sequence having at least about 80%, at least about 90%, at least about 95%, at least about 98% or at least about 99% sequence identity to any of the specific sequences disclosed herein.

In aspects and embodiments of the disclosure, there is provided an engineered vaccinia virus vector comprising one or more nucleic acid according to this disclosure; particularly, an isolated nucleic acid as disclosed herein.

In embodiments, the nucleic acid encoding a fusion polypeptide comprising a vaccinia virus envelope protein or part thereof and at least one complement regulatory protein or functional fragment thereof is inserted into the vaccinia virus vector at a locus outside the corresponding wild-type vaccinia virus envelope protein locus. For example, the fusion construct may be inserted into the thymidine kinase (TK) gene locus. In various embodiments, the TK gene is deleted or inactivated as a result of the insertion.

In some embodiments, the nucleic acid encoding the fusion polypeptide is operably linked to the native promoter for the corresponding vaccinia virus envelope protein.

Suitably, according to embodiments, the engineered vaccinia virus genome is selected from one of Copenhagen, Western Reserve, Wyeth, Lister or a modified Vaccinia Ankara strain. In some embodiments, the vaccinia virus strain is Copenhagen or Western Reserve strain; particularly the Copenhagen strain.

In aspects and embodiments, there is provided a modified vaccinia virus virion comprising a nucleic acid according to the disclosure.

In aspects and embodiments, there is provided a fusion polypeptide encoded by a nucleic acid according to the disclosure. Polypeptides according to the disclosure may have an amino acid sequence selected from any one of SEQ ID NOs: 47 to 56 or 63 to 68, or a sequence having at least about 90%, at least about 95%, at least about 98% or at least about 99% sequence identity thereto.

In aspects and embodiments, there is provided a pharmaceutical composition comprising a nucleic acid, an engineered vaccinia virus vector, a modified vaccinia virus virion or a polypeptide according to the disclosure, and a pharmaceutically acceptable carrier.

In various embodiments, the pharmaceutical compositions of this disclosure may be formulated for systemic, local or regional administration.

A pharmaceutical composition according to aspects and embodiments of the disclosure comprising a modified vaccinia virus virion may be formulated to provide a unit dose of: (i) between about 1×10³ and about 1×10¹⁵ pfu per ml; (ii) between about 1×10⁴ and about 1×10¹⁴ pfu per ml; or (iii) between about 1×10⁶ and about 1×10¹² pfu per ml.

The disclosure provides therapeutic compositions and methods for treating one or more diseases. In aspects and embodiments, there is provided an engineered vaccinia virus vector, an isolated nucleic acid, a modified vaccinia virus virion, or a pharmaceutical composition according to this disclosure for use in a method of treating cancer and/or proliferative diseases or disorders in a subject. Similarly, in aspects and embodiments, there is provided a method for treating cancer and/or proliferative diseases or disorders in a mammalian subject, the method comprising administering to the subject a therapeutically effective amount of the engineered vaccinia virus vector, an isolated nucleic acid, a modified vaccinia virus virion, or a pharmaceutical composition according to the disclosure.

In therapeutic uses and methods for treatment according to the disclosure, the cancer and/or proliferative diseases or disorders is selected from: lung cancers (e.g. lung adenocarcinomas), cervical cancer, breast cancer, cardiac cancer, colon cancer, prostrate cancer, brain glioblastoma, pancreatic cancer, leukemia (e.g. acute monocytic leukemia), lymphoma, kidney cancer, colorectal cancer, bladder cancer, testicular cancer, gastrointestinal cancer, liver cancer (e.g. hepatocarcinoma), and/or glioblastoma. The invention may also be useful in the treatment of one or more of skin cancer (e.g. melanoma), head and/or neck cancer, gallbladder cancer, uterine cancer, stomach cancer, tyroid cancer, laryngeal cancer, lip and/or oral cancer, throat cancer, ocular cancer and bone cancer. In embodiments, the cancer may be a primary cancer, a secondary cancer or a metastasis. In particularly beneficial embodiments, the cancer is a metastasis.

The compositions according to the disclosure may be administered to the subject systemically or locally. Suitable routes of administration may be selected from intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage or oral. In embodiments, the therapeutic compositions may be administered in combination with one or more additional therapeutic agent or therapy. When used, the additional therapeutic agent or therapy may be administered simultaneously, separately or sequentially to the administration of the therapeutic or medical composition of this disclosure.

It will be appreciated that any features of one aspect or embodiment of the invention may be combined with any combination of features in any other aspect or embodiment of the invention, unless otherwise stated, and such combinations fall within the scope of the claimed invention. Accordingly, within the scope of this disclosure, it is expressly intended that the various aspects, embodiments, examples and alternatives set out in the preceding paragraphs, in the clauses and in the claims and/or in the following description and drawings, and in particular the individual features thereof, may be taken independently or in any combination. That is, all embodiments and/or features of any embodiment can be combined in any way and/or combination, unless such features are incompatible. More particularly, it is specifically intended that any embodiment of any aspect may form an embodiment of any other aspect, and all such combinations are encompassed within the scope of the invention. The applicant reserves the right to change any originally filed claim or file any new claim, accordingly, including the right to amend any originally filed claim to depend on and/or incorporate any feature of any other claim although not originally claimed in that manner.

### BRIEF DESCRIPTION OF THE DRAWINGS

The invention is further illustrated by the accompanying drawings in which:

FIG. 1 Schematic representation of complement activation pathways and their regulation by engineered envelope proteins comprising of complement regulatory protein domains (ENV-CRP).

FIG. 2 Schematic representation of vaccinia virus envelope illustrating envelope proteins distribution and complexes.

FIG. 3 Sequence alignment of A13L proteins from various vaccinia viral species. Key: ref|YP_233014.1| (Vaccinia virus; SEQ ID No.: 1); ref|NP_570520.1| CMLV130 (Camelpox virus; SEQ ID No.: 2); ref|NP_042161.1| VARVgp117 (Variola virus; SEQ ID No.: 3); ref|NP_619928.1| CPXV145 protein (Cowpox virus; SEQ ID No.: 4); ref|YP_717441.1| Taterapox virus (SEQ ID No.: 5); ref|NP_536551.1| A14L (Monkeypox virus Zaire-96-I-16; SEQ ID No.: 6); ref|YP_009281879.1| (Volepox virus; SEQ ID No.: 7); ref|YP_010085590.1| (Akhmeta virus; SEQ ID No.: 8); ref|NP_671634.1| EVM116 (Ectromelia virus; SEQ ID No.: 9); ref|YP_010085800.1| putative A13L protein (Orthopoxvirus Abatino virus; SEQ ID No.: 10); ref|YP_009282825.1| (Skunkpox virus; SEQ ID No.: 11); ref|YP_009143440.1| (Raccoonpox virus; SEQ ID No.: 12); ref|YP_004821469.1| (Yokapox virus; SEQ ID No.: 13); ref|YP_009408310.1| (Murmansk poxvirus; SEQ ID No.: 14); ref|YP_009408512.1| NY_014 poxvirus (SEQ ID No.: 15); and ref|NP 938359.1| (Yaba monkey tumor virus; SEQ ID No.: 16) FIG. 4 Sequence alignment of A27L proteins from various vaccinia viral species. Key: P20535.1 Vaccinia Copenhagen virus A27 (SEQ ID No.: 17); YP_233032.1 Vaccinia virus A27 (SEQ ID No.: 18); NP_619946.1 CPXV162 Cowpox virus A27 (SEQ ID No.: 19); NP_570536.1 CMLV146 Camelpox virus A27 (SEQ ID No.: 20); NP_042178.1 VARVgp134 Variola virus (SEQ ID No.: 21); YP_010085815.1 Orthopox Abatino virus (SEQ ID No.: 22); YP_010085606.1 Akhmeta virus (SEQ ID No.: 23); NP_671648.1 EVM129 Ectromelia virus (SEQ ID No.: 24); YP_717458.1 Taterapox virus (SEQ ID No.: 25); NP_536566.1 A29L Monkeypox virus (SEQ ID No.: 26); YP_009281894.1 Volepox virus (SEQ ID No.: 27); YP_009143455.1 Raccoonpox virus (SEQ ID No.: 28); and YP_009282840.1 Skunkpox virus (SEQ ID No.: 29)

FIG. 5 Schematic representation illustrating the manufacture of various vaccinia virus expression cassettes for expressing fusion proteins of the disclosure from the A13L locus in vaccinia virus.

FIG. 6 Schematic representation illustrating the manufacture of various vaccinia virus expression cassettes for expressing fusion proteins of the disclosure from the J2R(TK) locus in vaccinia virus.

FIG. 7 Virus infectivity assay following human serum incubation. (A) Pre-incubation for 30 minutes or 60 minutes of parental control virus (WT; left hand columns, blue) or virus encoding an A13-CD55-V5 fusion protein according to an embodiment of the invention (vA13-CD55; right hand columns, red), with human serum. Data shows that, in contrast to WT, the engineered virus of the invention (vA13-CD55) has significantly increased resistance to serum-mediated neutralisation, exhibiting much greater infectivity following incubation with human serum. (B) Whole cell lysates of cells infected with WT or vA13-CD55 virus were probed with antibodies against the V5 affinity tag to reveal expression of the fusion protein.

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs (e.g. in cell culture, molecular genetics, nucleic acid chemistry and biochemistry).

Unless otherwise indicated, the practice of the present invention employs conventional techniques of chemistry, molecular biology, microbiology, recombinant DNA technology, chemical methods, pharmaceutical formulations and delivery and treatment of animals, which are within the capabilities of a person of ordinary skill in the art. Such techniques are also explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

In order to assist with the understanding of the invention several terms are defined herein.

The terms ‘nucleic acid’, ‘polynucleotide’, and ‘oligonucleotide’ are used interchangeably and refer to a deoxyribonucleotide (DNA) or ribonucleotide (RNA) polymer, in linear or circular conformation, and in either single- or double-stranded form. For the purposes of the present invention such DNA or RNA polymers may include natural nucleotides, non-natural or synthetic nucleotides, and mixtures thereof. Non-natural nucleotides may include analogues of natural nucleotides, as well as nucleotides that are modified in the base, sugar and/or phosphate moieties (e.g. phosphorothioate backbones). Examples of modified nucleic acids are PNAs and morpholino nucleic acids. Generally, an analogue of a particular nucleotide has the same base-pairing specificity, i.e. an analogue of G will base-pair with C. For the purposes of the invention, these terms are not to be considered limiting with respect to the length of a polymer.

A ‘gene’, as used herein, is the segment of nucleic acid (typically DNA) that is involved in producing a polypeptide or ribonucleic acid gene product. It includes regions preceding and following the coding region (leader and trailer) as well as intervening sequences (introns) between individual coding segments (exons). Conveniently, this term may also include the necessary control sequences for gene expression (e.g. enhancers, silencers, promoters, terminators etc.), which may be adjacent to or distant to the relevant coding sequence, as well as the coding and/or transcribed regions encoding the gene product.

As used herein, the term ‘vector’ is used to refer to a nucleic acid vector, e.g., a DNA vector, such as a plasmid, an RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in e.g. WO 1994/11026. Expression vectors of the invention may contain one or more additional sequence elements used for the expression of proteins and/or the integration of these polynucleotide sequences into the genome of a host cell, such as a mammalian cell (e.g., a human cell). Exemplary vectors that can be used for the expression of antibodies and antibody fragments described herein include plasmids that contain regulatory sequences, such as promoter and enhancer regions that direct gene transcription. Vectors may contain nucleic acids that modulate the rate of translation of a target gene or that improve the stability or nuclear export of the mRNA that results from gene transcription. These sequence elements may include, e.g., 5′ and 3′ untranslated regions, an internal ribosomal entry site (IRES), a ribosomal skipping sequence, such as picornavirus 2A sequences (T2A, F2A. E2A), and a polyadenylation signal site in order to direct efficient transcription of the gene carried on the expression vector. The vectors described herein may also contain a polynucleotide encoding a marker for selection of cells that contain such a vector. Examples of a suitable marker include genes that encode resistance to antibiotics, such as neomycin, geneticin, ampicillin, chloramphenicol, kanamycin, or nourseothricin.

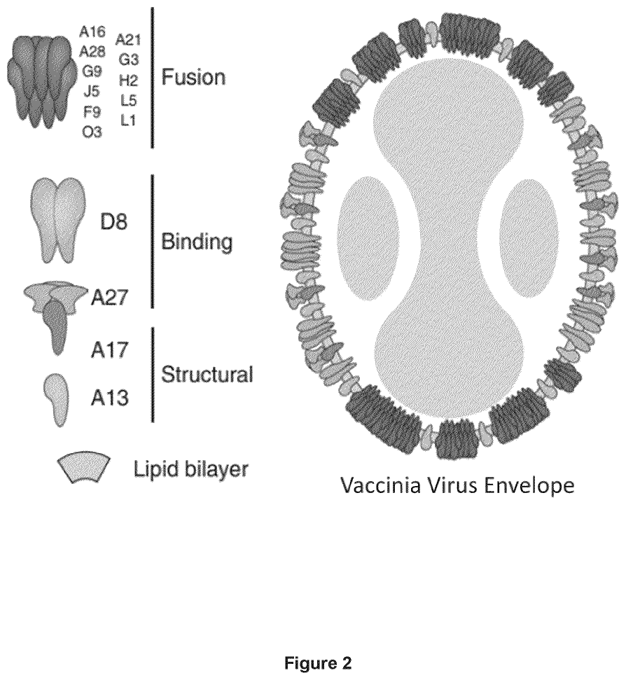

As used herein, ‘A13L’ refers to a vaccinia virus gene encoding a protein of approx. 70 amino acids that is one of the main components of the vaccinia virus membrane (FIG. 2 and see e.g. Unger & Traktman (2004), J Virol., 78(16): 8885-8901). It is thought to be essential for virion morphogenesis, playing a primary role in the transition of immature virions (IV) into intracellular mature virions (IMV). Examples of A13L in various species include: YP_233014.1 Vaccinia virus, NP_570520.1 CMLV130 Camelpox virus; NP_042161.1 VARVgp117 Variola virus; NP_619928.1 CPXV145 protein Cowpox virus; YP_717441.1 Taterapox virus; NP_536551.1 A14L Monkeypox virus Zaire-96-I-16; YP_009281879.1 Volepox virus; YP_010085590.1 Akhmeta virus; NP_671634.1 EVM116 Ectromelia virus; YP_010085800.1 putative A13L protein Orthopoxvirus Abatino; YP_009282825.1 Skunkpox virus; YP_009143440.1 Raccoonpox virus; YP_004821469.1 Yokapox virus; YP_009408310.1 Murmansk poxvirus; YP_009408512.1 NY_014 poxvirus; NP_938359.1 Yaba monkey tumor virus.

As used herein, ‘A27L’ refers to a vaccinia virus gene encoding a protein of approx. 110 amino acids that is one or the major component of the vaccinia virus membrane (FIG. 2 and see e.g. Vazquez et al., (1998), J Virol., 72(12): 10126-10137). It is located on the surface of the intracellular mature virus (IMV) form and is thought to be essential for both the release of extracellular enveloped virus (EEV) from the cells and virus spread. Examples of A27L in various species include: P20535.1 A27_VACCC RecName: Full=14 kDa fusion protein; YP_233032.1 IMV surface protein Vaccinia virus; NP_619946.1 CPXV162 protein Cowpox virus; NP_570536.1 CMLV146 Camelpoxvirus; NP_042178.1 hypothetical protein VARVgp134 Variola virus; YP_010085815.1 putative A27L protein Orthopoxvirus Abatino; YP_010085606.1 IMV surface protein Akhmeta virus; NP_671648.1 EVM129 Ectromelia virus; YP_717458.1 IMV surface protein Taterapox virus; NP_536566.1 A29L Monkeypox virus Zaire-96-I-16; YP_009281894.1 imv surface protein Volepox virus; YP_009143455.1 IMV surface protein Raccoonpoxvirus; YP_009282840.1 imv surface protein Skunkpoxvirus; YP_009408527.1 IMV surface protein NY_014 poxvirus; YP_009408325.1 IMV surface protein Murmansk poxvirus; YP_004821484.1 IMV surface protein Yokapox virus; YP_009408075.1 IMV membrane protein, fusion Eptesipox virus; YP_008658541.1 IMV-cell attachment, fusion, and microtubule transport Squirrelpox virus: YP_009389390.1 putative fusion protein-like protein Seal parapoxvirus; YP_009177162.1 A type inclusion-like/fusion protein Turkeypox virus; YP_009112843.1 putative fusion protein Parapoxvirus red deer/HL953; YP_009268837.1 imv surface protein, fusion protein Pteropox virus; NP_044084.1 MC133L Molluscum contagiosum virus subtype 1; YP_009480655.1 IMV surface protein Sea otter poxvirus; NP_957881.1 ORF104 fusion protein Orf virus; YP_010085255.1 P4c precursor Western grey kangaroopox virus; YP_010085419.1 P4c precursor Eastern grey kangaroopox virus; NP_955288.1 CNPV265 A type inclusion-like/fusion protein Canarypox virus; YP_009046422.1 A-type inclusion protein Pigeonpox virus; YP_009046188.1 A-type inclusion protein Penguinpox virus; YP_009448114.1 A-type inclusion protein Flamingopox virus FGPVKD09; NP_039154.1 A type inclusion protein Fowlpox virus; NP_958013.1 ORF104 fusion protein Bovine papular stomatitis virus; YP_005296322.1 MV attachment protein Cotia virus SPAn232; YP_003457410.1 viral fusion peptide Pseudocowpox virus; YP_009329750.1 putative fusion protein BeAn 58058 virus; NP_570274.1 Hypothetical protein SWPVgp114 Swinepox virus; NP_150551.1 Hypothetical protein LSDVgp117 Lumpy skin disease virus NI-2490; NP_073502.1 117L protein Yaba-like disease virus; YP_227501.1 Fusion protein Deerpox virus W-848-83; NP_938372.1 Hypothetical protein YMTVg 117L Yaba monkey tumor virus; NP_052004.1 Hypothetical protein SFV_s115L Rabbit fibroma virus; NP_659689.1 Hypothetical protein SPPV_112 Sheeppox virus; YP_001293308.1 Hypothetical protein GTPV_gp112 Goatpox virus Pellor; NP_051829.1 Hypothetical protein MYXV_gp119 Myxoma virus.

As used herein, ‘VCP’ refers to a vaccinia virus gene encoding a protein of approx. 243-amino acids, which is the major protein secreted from and expressed on the surface of vaccinia virus-infected cells (Girgis et al., (2008), J Virol., 82(9): 4205-4214). It is similar in sequence to the regulators of complement activation, and is homologous to the smallpox inhibitor of complement enzymes (SPICE) encoded by variola virus and to the monkeypox inhibitor of complement enzymes (MoPICE) (Liszewski et al., (2006), J Immunol., 176(6): 3725-3734). Its role is to defend the virus against attack by the host complement system by inhibiting complement produced through both the classical and alternative pathways (FIG. 2 and see e.g. Sahu et al., (1998), J Immunol., 160(11): 5596-5604). Examples of VCP in various species include: YP_232907.1 secreted complement binding C3b/C4b C3L Vaccinia virus: NP_570413.1 secreted complement-binding protein Camelpox virus: NP_042056.1 secreted complement-binding protein D15L B19L SPICE Variola virus: NP_619823.1 CPXV034 Cowpox virus: NP_671535.1 secreted complement-binding protein Ectromelia virus: YP_010085695.1 putative C3L protein Orthopoxvirus Abatino: YP_010085478.1 complement binding protein Akhmeta virus: NP_536444.1 D14L MOPICE Monkeypox virus Zaire-96-I-16: YP_009282718.1 complement binding Skunkpox virus: YP_009281772.1 complement binding Volepox virus: YP_009143334.1 Secreted complement binding protein C3b/C4b Raccoonpox virus: YP_717333.1 secreted protein Taterapox virus: YP_005296214.1 complement binding protein Cotia virus SPAn232: YP_009408407.1 complement binding NY_014 poxvirus: YP_009408205.1 complement binding Murmansk poxvirus; NP_051858.1 m144R Myxoma virus.

As used herein, ‘a ‘herpes virus complement regulatory protein’ is a complement regulatory protein (similar to VCP) expressed by a herpes virus, such as: NP_570746.1 complement binding protein Macacine gammaherpesvirus 5; NP_040205.1 complement control protein homologue Saimiriine gammaherpesvirus 2; YP_010084365.1 ORF4 Retroperitoneal fibromatosis-associated herpesvirus; NP_040206.1 complement control protein homologue Saimiriine gammaherpesvirus 2; NP_047979.1 complement control protein homolog ccph Ateline gammaherpesvirus 3; YP_010084544.1 ORF4 Macaca nemestrina rhadinovirus 2; YP_010084543.1 ORF4A Macaca nemestrina rhadinovirus 2; YP_009551812.1 hypothetical protein Rhinolophus gammaherpesvirus 1; YP_238307.1 JM4 Macaca fuscata rhadinovirus; YP_001129351.1 ORF4; KCP Human gammaherpesvirus 8; YP_009408125.1 Secreted complement binding protein C3b/C4b Eptesipox virus; YP_009229839.1 complement control protein-like protein Myotis gammaherpesvirus 8; YP_009552470.1 Complement control protein Eptesicus fuscus gammaherpesvirus; NP_044845.1 complement regulatory protein Murid gammaherpesvirus 4; YP_010085882.1 complement control protein Wood mouse herpesvirus; YP_004207839.1 complement regulatory protein Cricetid gammaherpesvirus 2; YP_004207845.1 complement regulatory protein Cricetid gammaherpesvirus 2.

The term ‘amino acid’ in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The term ‘peptide’ as used herein (e.g. in the context of a viral envelope protein or fusion protein of this disclosure) refers to a plurality of amino acids joined together in a linear or circular chain. term oligopeptide is typically used to describe peptides having between 2 and about 50 or more amino acids. Peptides larger than about 50 amino acids are often referred to as polypeptides or proteins. For purposes of the present invention, however, the term ‘peptide’ is not limited to any particular number of amino acids, and is used interchangeably with the terms ‘polypeptide’ and ‘protein’.

As used herein, the terms ‘chimeric’ or ‘fusion’ in the context of a polypeptide/protein are used interchangeably to refer to a polypeptide sequence, which comprises at least a first and a second polypeptide sequence that are covalently joined to one another (e.g. C-terminal to N-terminal), and wherein the first and second polypeptide sequences do not occur in nature within the same polypeptide/protein. The first and second polypeptide sequences may each be a single protein domain or may comprise more than one protein domain that combine to perform a function. Typically, such first and second polypeptide sequences are joined to one another by an amino acid/peptide linker which may have any suitably length, but is typically between 5 and 50 amino acids. Beneficially, such a peptide linker is relatively inert, i.e. it does not interfere with the function or the fusion protein or with the function of the first and second polypeptide sequences, and may comprise a predominance of Gly and/or Ser residues. Correspondingly, the terms ‘chimeric’ or ‘fusion’ may also be used to refer to a nucleic acid/polynucleotide sequence that encodes a chimeric or fusion polypeptide/protein.

A ‘complement regulator protein’ or ‘complement regulatory protein’ is a protein that plays a regulatory role in humans and animals to ensure that the complement system of (innate) immunity does not become over-activated, thus causing harm to self-tissues. There are several soluble regulatory proteins such as C1 inhibitor, C4b binding protein, and factors H, B, D, and I. In addition, membrane bound complement regulatory proteins (mCRPs) provide another complement control mechanism that includes CD35 (Complement receptor 1, CR1), CD46 (membrane cofactor protein, MCP), CD55 (decay acceleration factor, DAF), and CD59 (protectin). Complement regulatory proteins are expressed on every cell in the human body, though the expression of these mCRPs varies across tissue type. Since different tissues face different immune interactions within the body, it has been hypothesised that mCRP expression across tissue types may also be variable (Qin et al., (2001), Mamm. Genome, 12:582-589).

In the context of the present disclosure, the terms ‘individual’, ‘subject’, or ‘patient’ are used interchangeably to indicate an animal that may be suffering from a medical (pathological) condition and may be responsive to a molecule, composition, method, use, medical treatment or therapeutic treatment regimen of the disclosure. The animal is suitably a mammal, such as a human, non-human primate, cow, sheep, pig, dog, cat, rabbit, bat, mouse or rat. In particular, the subject may be a human, a rabbit or a mouse; and especially a human.

The viruses, nucleic acids, compositions or agents of the disclosure may be used to treat one or more diseases, infections or disorders. The terms ‘treat’, ‘treating’ or ‘therapy’ as used herein in the context of a disease state or condition, refer to a reduction in severity of the disease or condition or pathogenic symptom, such that the therapy is effective in curing, inhibiting, alleviating, reducing or preventing the adverse effects of the diseases or disorders to be treated, or is sufficient to achieve a physiological or biochemically-detectable effect. Thus, an ameliorative, inhibitory or preventative effect in relation to disease or disorder may be achieved. In various embodiments, such treatment may involve oncolysis or killing of tumour cells, inhibiting the growth or metastases or tumour cells, decreasing tumour size, and/or otherwise reversing or reducing the malignant phenotype of tumour cells. For example, in the treatment of cancer, tumor growth may be reduced by up to about 100%, by up to about 90%, up to about 80%, up to about 70%, up to about 60%, up to about 50%, up to about 40%, up to about 30%, up to about 20% or up to about 10%. In embodiments, tumour growth may be reduced by between about 10% and about 90%, between about 20% and about 80% or between about 30% and about 70%. Accordingly, in some embodiments, the viruses, nucleic acids, peptides or compositions (i.e. therapeutic agents according to the disclosure) may be manufactured into medicaments or may be incorporated or formulated into pharmaceutical compositions.

### Vaccinia Virus

Vaccinia virus is a member of the Orthopoxvirus or Poxviridae family, the Chordopoxvirinae subfamily, and the Orthopoxvirus genus. The Orthopoxvirus genus is more homogeneous than other members of the Chordopoxvirinae subfamily and includes 11 distinct but closely related species, which includes vaccinia virus, variola virus (causative agent of smallpox), cowpox virus, buffalopox virus, monkeypox virus, mousepox virus and horsepox virus species as well as others (see Moss, 1996). Certain embodiments of the invention, as described herein, may be extended to other members of Orthopoxvirus genus as well as the Parapoxvirus, Avipoxvirus, Capripoxvirus, Leporipoxvirus, Suipoxvirus, Molluscipoxvirus, and Yatapoxvirus genus. A genus of the Chordopoxviridae subfamily is generally defined by serological means, including neutralisation and cross-reactivity in laboratory animals.

Vaccinia virus is a large, enveloped virus having a linear double-stranded DNA genome of about 190 kbp and encoding for approximately 250 genes. Vaccinia, unusually for a DNA virus, replicates only in the cytoplasm of the host cell and, therefore, it is a non-integrative vector. The vaccinia genome encodes the enzymes and proteins necessary for viral DNA replication. During replication, vaccinia produces several infectious forms which differ in the composition of their outer membranes: the intracellular mature virion (IMV, also known as the mature virion, MV), the intracellular enveloped virion (IEV), the cell-associated enveloped virion (CEV), and the extracellular enveloped virion (EEV, also known as the extracellular virion, EV). IMV is the most abundant infectious form and is thought to be responsible for spread between hosts. On the other hand, the CEV is believed to play a role in cell-to-cell spread and the EEV is thought to be important for long range dissemination within the host organism.

### Vaccinia Virus Immunotherapy

Advantages of using vaccinia virus in therapy include that vaccinia vectors may carry up to 25 kb of foreign DNA without the need for viral deletions; they have a broad host range that permits the infection of primary cultures and many different cell lines; they exhibit cytoplasmic replication; and that the viral genome does not splice its primary transcripts. Replication of the virus in host cells leads to cell lysis on viral release.

The use of vaccinia viruses as clinical viral immunotherapy vectors demonstrates promise for cancer treatment, but as yet, only modest clinical success has been achieved due to the lack of efficient systemic delivery and virus spread. Typically, viral immunotherapy vectors can either be: (a) administered systemically through intravenous injection; or (b) locally through inoculating in the vicinity of the tumour (‘locoregional’ delivery) or directly injected into the tumour (‘intratumoral delivery’). Systemic delivery of the virus would be of benefit to treat both the primary tumour and disseminated, potentially undiagnosed, metastatic deposits simultaneously. However, viral immunotherapy is stymied by poor activity following systemic delivery, which limits efficacy to tumours that are accessible by local injection. This rapid inactivation is primarily a result of the innate immune response in non-immunised and antigen naïve individuals. The complement system is a major orchestrator of the innate immune response, being a critical defence mechanism against pathogens, recognising and opsonising viral membranes that leads to recognition by immune cells or virolysis. Enveloped RNA and DNA viruses are particularly sensitive to the action of the complement cascade.

As metastases are the major cause of death in cancer patients, a better solution is needed to avoid inactivation in the bloodstream so that the virus can reach deep into the vasculature of primary tumours or can reach the disseminated metastases. As such, methods that can sufficiently control this inactivation of vaccinia virus will allow improved delivery of active therapeutic viruses to target cells within tumours and will greatly benefit the treatment of cancer. Further, intratumoral delivery is complicated by the need to image and locate precisely the diseased tissue prior to injection, and the high interstitial pressure within the tumour, which can hamper delivery locally via a needle and leaking out at point of injection. Improved vaccinia therapies may therefore be achieved with modified vaccinia viruses that can avoid the host immune system in conjunction with the convenience of systemic delivery and improved activity of the treatment.

Accordingly, there is an acute need for engineered vaccinia viruses that are more robust against complement attack and can therefore be delivered systemically and spread more readily from the point of infection within the tumour and to distal metastases. The present invention addresses this need and provides a solution to the systemic delivery of vaccinia virus through modification of the viral proteins—particularly viral envelope proteins—to enhance tumour-targeted systemic delivery of the virus, intratumoral and intertumoral spread of the virus and enhanced tumour-specific replication of the virus. Advantageously, the modified, engineered virus of this disclosure can be utilised as a platform vector for systemic delivery for viral immunotherapy.

Many of the poxvirus genomes, including those of different strains of vaccinia virus, have been sequenced. The genome of the vaccinia virus Western Reserve (WR) strain contains 218 potential open reading frames. Analysis of the proteins in the intracellular mature virion (IMV) showed that it contains at least 81 viral proteins, including structural proteins, enzymes, transcription factors, including A2.5L, A3L, A4L, A5R, A6L, A7L, A9L, A10L, A12L, A13L, A14L, A14.5L, A15L, A16L, A17L, A18R, A21L, A22R, A24R, A25L, A26L, A27L, A28L, A29L, A30L, A31R, A32L, A42R, A45R, A46R, B1R, C6L, D1R, D2R, D6R, D7R, D8L, D11L, D12L, D13L, E1L, E4L, E6R, E8R, EI OR, E11L, F8L, F9L, F10L, F17R, G1L, G3L, G4L, G5R, G5.5R, G7L, G9R, H1L, H2R, H3L, H4L, H5R, H6R, I1L, I2L, I3L, I5L, I6L, I7L, I8R, J1R, J3R, J4R, K4L, L1R, L3L, L4R, L5R, 02L. Among these are important proteins involved in binding, virion fusion and structural integrity. Those of known function include: 4 attachment proteins A27, H3, D8, A26; entry fusion complex (EFC) of 11 components A16, A21, A28, G3, G9, H2, J5, L5, O3, L1 and F9 and structural proteins such as A13. Proteins encoded by A27L, H3L, L1R, and D8L have been identified as major immunogenic proteins and effort has concentrated on the engineering of these proteins for avoidance of neutralisation by the adaptive immune system (see e.g. WO 2020/086423 A1). Proteins A27, H3, and D8 are adhesion molecules that bind to host glycosaminoglycans (GAGs) heparan sulfate (HS) and chondroitin sulfate (CS) and mediate endocytosis of the virus into the host cell. L1 protein is involved in virus maturation.

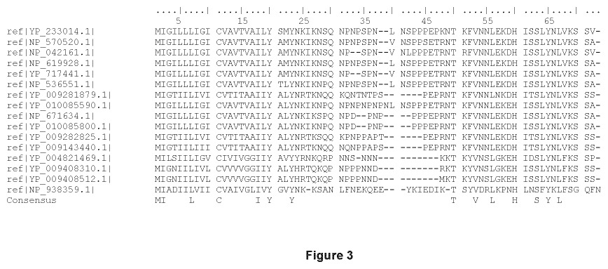

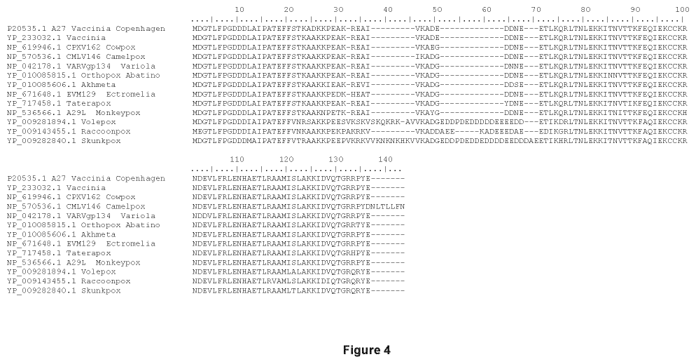

Various members of the Orthopoxvirus genus, as well as other members of the Chordovirinae subfamily show conservation in their envelope proteins as shown herein (see FIGS. 3 and 4 showing sequence alignments of A13 and A27, respectively).

The EEV form of the virus is released early in infection and has an additional membrane to that of the mature virion that helps shield the virus from complement by sequestering host complement proteins CD46, CD55, CD59 (Vanderplasschen et al. (1998) PNAS) as well as other proteins including MHCI. A56 on the EEV envelope can also disulphide bond with the secreted vaccinia complement control protein VCP to further reduce complement-mediated destruction of the virus. This is believed to provide the virus with the ability to spread more widely and infect new tissues. In contrast, the IMV does not have any innate or acquired ability to avoid complement and modifications of the viral envelope or proteins associated with the envelope can potentially affect packaging and reduce infectivity of the virus for target cells. As a consequence, many attempts at modification of these proteins have failed to generate viable viruses (Paul et al., (2007), Viral Immunol.).

The present disclosure provides engineered vaccinia virus having improved ability to evade the immune system, e.g. the innate immune system of a host animal subject. The present disclosure encompasses any oncolytic vaccinia viral strain which has been suitably engineered. Potential vaccinia viruses genomes that may be used in accordance with the disclosure include Abatino macacapox virus, Akhmeta virus, Camelpox virus 903, Camelpox virus CMG, Camelpox virus CMS, Camelpox virus CP1, Camelpox virus CP5, Camelpox virus M-96, Cowpox virus (Brighton Red), Cowpox virus (strain GRI-90), Cowpox virus (strain Hamburg-1985), Cowpox virus (strain Turkmenia-1974), Elephantpox virus, Belo Horizonte virus, Ectromelia virus ERPV, Ectromelia virus Moscow, Ectromelia virus Naval, Ectromelia virus WH, Callithrix jacchus orthopoxvirus, Monkeypox virus (strain Sierra Leone 70-0266), Monkeypox virus (strain Zaire 77-0666), Monkeypox virus Zaire-96-I-16, Raccoonpox virus, Skunkpox virus, Taterapox virus, Aracatuba virus, Buffalopox virus, Cantagalo virus, Guarani P1 virus, Guarani P2 virus, Horsepox virus, Modified Vaccinia Ankara virus, Rabbitpox virus, Rabbitpox virus Utrecht, SPAN 232 virus, Vaccinia Virus Acambis 3000 MVA, Vaccinia virus Ankara, Vaccinia virus Copenhagen, Vaccinia virus Dairen I, Vaccinia virus GLV-1h68, Vaccinia virus IHD-J, Vaccinia virus L-IPV, Vaccinia virus LC16M8, Vaccinia virus LC16MO, Vaccinia virus Lister, Vaccinia virus LIVP, Vaccinia virus Mariana, Vaccinia virus Tashkent, Vaccinia virus Tian Tan, Vaccinia virus WAU86/88-1, Vaccinia virus Western Reserve, Vaccinia virus WR, Vaccinia virus WR 65-16, Vaccinia virus Wyeth, Variola major virus, Variola minor virus, Variola virus human/India/Ind3/1967, Volepox virus, Alaskapox virus, Cetacean poxvirus 1, Dolphin poxvirus 1, Cetacean poxvirus 2, Cowpox-Vaccinia virus, Feline poxvirus ITA1_PG, Feline poxvirus ITA2_BC, Orthopoxvirus GCP2010, Orthopoxvirus GCP2013, Orthopoxvirus NY99014/1999, Orthopoxvirus OH08/2008, Orthopoxvirus Tena Dona, Orthopoxvirus VPXV_CA85, Orthopoxvirus WA01960/2001, Steller sea lion poxvirus, Orthopoxvirus sp (described in the NCBI taxonomy database).

In some embodiments, the vaccinia virus for use in accordance with the disclosure is based on the Copenhagen, Western Reserve, Wyeth, Lister or Modified Vaccinia Ankara strain. Beneficially, the genetically modified virus is based on a Copenhagen or Western Reserve viral strain, and particularly is a Copenhagen-derived vaccinia virus. Any such engineered virus may suitably be provided for use and in methods for the treatment of various cancers. The vaccinia viruses described herein can be administered to a patient, such as a mammalian patient (e.g., a human patient) to treat a variety of cell proliferation disorders, including a wide range of cancers.

### Modified Vaccinia Viruses

The study of vaccinia virus and modifications of the vaccinia virus genome have identified a large number of engineered/modified viruses that show benefits in cancer therapy.

Whilst wild type vaccinia virus has no tumour selectivity, viral strains have been modified for selectivity towards cancer cells through inactivating mutations or deletions of genes such as viral thymidine kinase (gene J2R) or ribonucleotide reductase (F4L).

For example, engineered vaccinia viruses have been identified that exhibit one or more of markedly improved oncolytic activity, replication in tumours, infectivity, immune evasion, tumour persistence, capacity for incorporation of exogenous DNA sequences, and amenability for large scale manufacturing when the viruses are engineered to contain deletions in one or more, or all, of the vaccinia viral genes: C2L, C1L, N1L, N2L, M1L, M2L, K1L, K2L, K3L, K4L, K5L, K6L, K7R, J2R, F1L, F2L, F3L, B14R, B15R, B16R, B17L, B18R, B19R, B20R, K ORF A, K ORF B, B ORF E, B ORF F, B ORF G, B21R, B22R, B23R, B24R, B25R, B26R, B27R, B28R, and B29R (see for example, WO 2019/134049). Aspects and embodiments of the present disclosure, therefore, relate to engineered vaccinia viruses or viral nucleic acid genomes comprising the deletion of one or more of the above genes; i.e. wherein various wild-type vaccinia virus genes have been deleted to enhance the oncolytic activity of the vaccinia virus. In various embodiments, at least one of the above genes is deleted from the recombinant vaccinia virus genome. In various embodiments, at least 2, 3, 4, 5, 6, 7, 8, 9, 10 or more of the above genes are deleted from the recombinant vaccinia genome.

In various embodiments, the modified vaccinia virus contains at least the deletion of the B8R gene. The vaccinia virus B8R gene encodes a secreted protein with homology to gamma interferon receptor (IFN-g). In vitro, the B8R protein binds to and neutralises the antiviral activity of several species of gamma inteterferon including human and rat gamma interferon. While inactive in mice, deleting the B8R gene prevents the impairment of IFN-gamma in humans. Deletion of the B8R gene thus results in enhanced safety without a concomitant reduction in immunogenicity.

It will be appreciated that where an engineered virus or viral genome of this disclosure comprises a deletion of one or more endogenous gene, one or more heterologous gene, expression construct or polynucleotide sequence may be inserted into the genome in its place, for example, through homologous recombination or any other suitable mechanism, as described elsewhere herein.

Various beneficial modifications to vaccinia virus for use in oncolytic therapy are known to the person skilled in the art, some of which will be described in this disclosure; but any other modifications that may be useful and apparent to the skilled person in the art are also envisaged herein.

Several current clinical studies testing vaccinia virus as an oncolytic virus harbor deletions in the viral Thymidine Kinase (TK) gene. This deletion attenuates the virus, rendering the virus dependent upon the activity of cellular thymidine kinase for DNA replication and viral propagation. Cellular thymidine kinase is expressed at a low level in most normal tissues but is expressed at elevated levels in many cancer cells. Through such metabolic targeting, TK-viruses can grow efficiently in cells that have a high metabolic rate (e.g., healthy cells or tumor cells) and will not growwell in cells that have low levels of thymidine kinase. Since some tumour cells are quiescent, however (e.g., cancer stem cells), some TK-viruses may be unable to kill all populations of cancer cells. Indeed, many chemotherapic drugs are also largely ineffective against such quiescent cells.

Therefore, in some embodiments, the engineered vaccinia viral vectors and viruses of the disclosure may lack the TK gene or to disable or render inactive the TK gene to promote activity (e.g. preferential replication) against rapidly dividing cells (particularly cancer cells); whereas in some alternative embodiments, the engineered viral vectors and viruses of the disclosure may include TK in order to allow propagation/replication in quiescent cancer cells.

In further embodiments, the oncolytic vaccinia virus may be engineered to also (or in some embodiments, alternatively) lack vaccinia virus growth factor (VGF). VGF is a secreted protein produced early in the infection process, which acts as a mitogen to prime surrounding cells for infection. Thus, in some embodiments, the oncolytic vaccinia virus may be engineered to lack both VGF and TK activity.

Deletions have also been created in several other vaccinia virus genes, such as ribonucleotide reductase (F4L or RR). In fact, double (or triple) deletions of viral genes alongside TK such RR and/or VGF, have been shown to confer a strict vaccinia virus tumour specificity.

Various different modified vaccinia virus strains have been developed and may provide the vaccinia viral genome on which the present invention is based. Clinical and preclinical stage examples of suitable vaccinia viruses include, for example: Pexa-Vec (JX-594), a TK deleted Wyeth strain; vvDD a TK VGF deleted Western Reserve strain; LV-1 h68 a Lister strain with TK, F14.5 L and A65R deleted; VG9-GMCSF a TK deleted Tiantan Guang9 strain; ΔF4LΔJ2R, a Western Reserve strain in which F4L and TK are deleted; CVV a Wyeth derived strain isolated through repeated selection where TK is deleted; deVV5 a chimeric virus of strains Wyeth, Modified Ankara, Western Reserve, and Copenhagen where TK has been deleted. Other suitable candidate vaccinia viruses include those disclosed in e.g. US 20190218522 A, such as CF33 virus.

In various embodiments, the oncolytic vaccinia virus may be engineered to lack one or more genes involved in evading host interferon (IFN) response such as E3L, K3L, B18R, or B8R. B18R is known to neutralise secreted type-I IFNs. Other modifications to modify anti-apoptotic viral genes, are deletion of SPI-1 and/or SPI-2. Attenuation of virus for use as a cancer selective oncolytic agent include deletion of A56R (hemaglutinnin) in a background of F14.5 L and J2R in the Lister strain. Strategies to accelerate mRNA decay by removing de-capping enzymes D9 and D10 have also been used to limit activation of host defence. Combined deletions, Western Reserve virus Delta4 (A48R, B18R, C11R, and J2R), have also been created that act in tandem on metabolic, proliferation, and signaling pathways (see e.g. Guo et al. (2019), J. Immunotherapy Cancer, 7, 6).

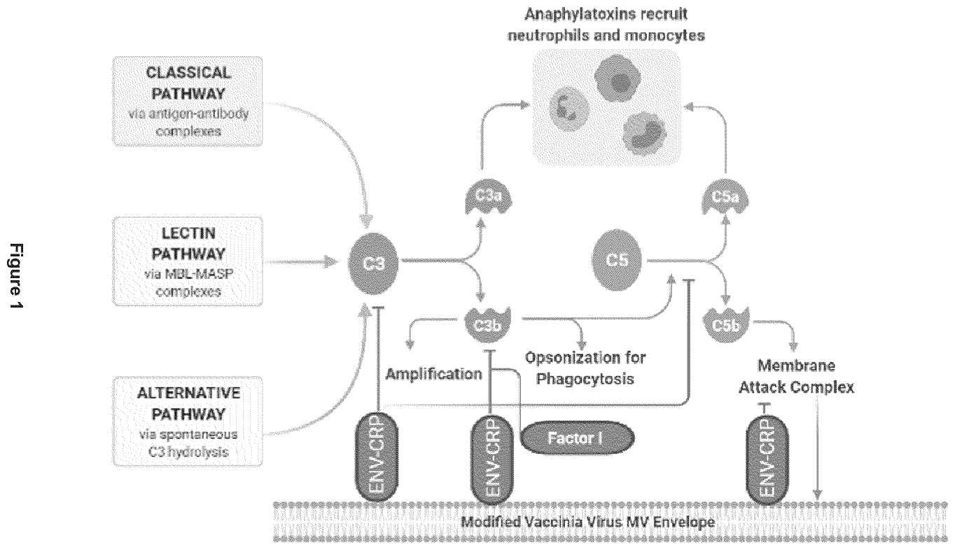

Vaccinia Virus Modifications to Overcome Complement-Mediated Neutralisation Complement is a key component of the innate immune system, targeting the virus for neutralisation and clearance from the circulatory system (FIG. 1). Complement enhances the potency of antibodies as demonstrated by diminished neutralisation against smallpox in the absence of complement. Therefore, complement is critical in the natural immune response against vaccinia virus. Complement component C1 recognises the Fc region of antibodies that are bound to viral epitopes and activates an enzymatic cascade of proteins which results in the formation of the C3 convertase C4b2b and cleavage of C3 and deposition of opsonic C3b fragments on surfaces. Alternatively, C3 might be activated spontaneously through hydrolysis of its internal thioester bond and reacting with a hydroxyl or amino group on the surface of a pathogen. The bound C3b serves as an opsonin for phagocytic cells and as a component of C3 convertase enzyme C3bBb (the Alternative Pathway for complement activation). Further cleavage and binding of C3b leads to formation of C5 convertase, cleavage of C5, and assembly of the membrane attack complex (C5b, 6, 7, 8, 9), which disrupts lipid bilayers such as the vaccinia envelope.

C3b can bind to normal cell membranes and unchecked activation can lead to inappropriate inflammation. Therefore, there are mechanisms that animals possess to regulate complement activation. In humans, complement can be negatively regulated by several membrane regulators of complement activation (RCA). RCAs downregulate complement activation in different ways—through inhibiting the formation and accelerating decay of C3 convertases by CD35 (complement receptor 1) and CD55 (decay-accelerating factor); by catabolising C3b and C4b through the action of CD35 and CD46, (membrane cofactor protein) acting as cofactors for regulatory proteins, Factor H and Factor I, to inhibit formation of C3 convertases C4b2a and C3bBb, and by preventing the formation of the membrane attack complex through CD59 activity. The extracellular enveloped vaccinia virus (EEV) is resistant to complement by sequestering these proteins in its viral envelope and it has been reported that incorporation of CD55 onto A27 of the IMV offers some complement protection.

The 14 kDa A27 protein binds to the HS receptor on a host cell surface via its N-terminal domain (residues 21 to 30) and is attached to the vaccinia virus envelope by interacting with the envelope protein A17 through its C-terminal domain (see e.g. FIG. 2). However, A27 has multiple roles in the virus lifestyle; in particular, A27 is involved in virus binding, fusion, and the intracellular transport and wrapping of IMVs. While A27L deletion mutants are not defective in IMV production, their EFC loses its polarisation. This likely underlies why such deletion variants are 8-fold less capable of mediating cell-cell fusion than wild type (WT) virions (Gray et al., 2019)

In view of the multiple and diverse roles that A27 plays in vaccinia virus life cycle, any changes to the encoded protein may be detrimental to one or more vaccinia virus functions.

However, it has been surprisingly found that certain mutations of A27, as disclosed herein, can provide for beneficial effects is complement avoidance while at the same time maintaining suitable levels of virus growth and/or infectivity. Thus, this disclosure provides fusion/chimeric proteins (and encoding nucleic acids) comprising complement inhibitors with A27, which beneficially may not be detrimental to A27 function and, as such, do not lead to loss of viral infectivity or undesirable aggregation. Various embodiments of the disclosure provide a modified native vaccinia virus complement control protein VCP fused at the N-terminus of A27, and to encoding nucleic acid molecules, particularly encoding nucleic acids or genes that are operably linked to/under the control of a native A27L promoter. Furthermore, the disclosure provides engineered viral genomes and engineered viral particles comprising such fusion/chimeric A27L genes. Such engineered vaccinia viruses may demonstrate greater inhibition of complement and/or improved infectivity relative to engineered vaccinia virus expressing A27-CD55 fused viral proteins.

Aspects and embodiments of this disclosure also relate to fusion/chimeric proteins (and encoding nucleic acids) comprising complement inhibitors with the A13L gene. Thus, there is provided a native or modified vaccinia virus complement control protein VCP (Liszewski et al., (2009), J. Immunol., 183: 3150-3159) fused with the A13 protein to chreate a chimeric protein. A13 is a structural protein, encoded by the A13L late gene, which is an integral component of the mature virion membrane. In view of the wide-spread/ubiquitous location of the A13 protein on the surface of vaccinia virus (FIG. 2), the inventors believe that there is no clear polarity in its location in the virion envelope, and as such, this presents an opportunity to broadly decorate the IMV envelope with complement control proteins whilst leaving the proteins involved in membrane fusion and binding unaffected. In order to optimise the physiological effect of such chimeric proteins in conjunction with the activity of a mature virion expressing such a fusion protein, VCP may conveniently be fused towards or at the N-terminus or towards or at the C-terminus of A13, depending on the desired effect. In this regard, the inventors have surprisingly found that A13 can accommodate a fusion with VCP, as described herein, and that both active components of the fusion are able to perform their intended, natural function. In particular, the fused VCP is able to bind to C3 and inhibit activity of C3 convertase. This disclosure, therefore, also encompasses nucleic acid molecules, particularly nucleic acids or genes that are operably linked to/under the control of a native A13L promoter, which encode an A13-VCP (or VCP-A13) fusion protein, such as those described herein. In addition, the disclosure provides engineered viral genomes and engineered viral particles comprising corresponding fusion/chimeric A13L genes.

In some suitable embodiments, the open reading frame of the complement control protein is fused towards the 3′ end of the A13L gene (corresponding to the C-terminus of the expressed protein) and the resultant fusion construct is flanked on either side by natural viral genone sequences, e.g. with the A14L gene and A13L promoter sequences at its 5′ end and the A12L gene at its 3′ end.

The present invention further provides recombinant vaccinia virus nucleic acids, genomes and virions comprising a heterologous nucleic acid encoding a complement activation modulator such as compstatin, CD55, CD59, CD46, CD35, factor H, C4-binding protein, CD35, VCP, Kaposi-sarcoma associated herpesvirus Kaposica/KCP, Herpesvirus saimiri (HVS) CCPH and HVS-CD59, Rhesus rhadinovirus RCP-H and RCP-1, murine gammaherpesvirus 68 (yHV-68) RCA, Influenzavirus M1, SPICE, MOPICE, EMICE or IMP, or modified sequences thereof. Expression of the complement activation modulators from viral genetic material in a host cell, for example, may provide the recombinant vaccinia viruses with the ability to regulate complement activation in a host/subject, and reduce complement-mediated virus neutralisation as compared to the wild-type virus. In some embodiments, the heterologous nucleic acid carried by an engineered/recombinant vaccinia virus according to this disclosure encodes a domain of human CD55, CD59, CD46, CD35, factor H, C4-binding protein, CD35 or VCP, or active modified sequences therefore, or other identified complement activation modulators. In some embodiments, the heterologous nucleic acid encodes a VCP or modified VCP protein (SEQ ID NO: 30 and SEQ ID NO: 31) or another poxvirus complement control protein such as SPICE (SEQ ID NO: 32, Variola virus VCP, encoded by D15L gene), MOPICE (SEQ ID NO: 33, Monkeypox virus Zaire-96-I-16 VCP encoded by D14L gene), EMICE (SEQ ID NOs: 75, 76; Ectromelia virus (ECTV) Moscow strain ECTV-MOS between nucleotides 27,564 and 26,776; Chen et al. (2003) Virology, 317: 165-186) or IMP (SEQ ID NOs: 77, 78; CPV-IMP protein from cowpox, as described by Miller et al. (1997) Virology, 229:126-133).

### Genetic Modification of Vaccinia Virus

Methods for the insertion or deletion of nucleic acids in or from a target genome include those described herein and known in the art.

In accordance with the disclosure, an endogenous, target viral gene can be mutated through recombination using a shuttle vector which encodes the modified protein and approximately 250-300 bp of homologous sequence flanking either side of the modification leading to homologous recombination.

Alternative methods for the insertion or deletion of nucleic acids from a target genome include CRISPR, zinc fingers mediated gene editing, TALENS etc.

In other embodiments, mutations may be produced and selected by processes of natural selection under selective pressure to promote and identify desired phenotypes.

Any suitable method known to the person skilled in the art may be used for nucleic acid delivery to effect expression of nucleic acids and/or virus genomes in accordance with the present disclosure. For example, any appropriate method by which a nucleic acid (e.g., DNA or RNA, including viral and non-viral vectors) can be introduced into an organelle, a cell, a tissue or an organism may be used. Such methods include, but are not limited to, direct delivery of nucleic acid by injection (U.S. Pat. Nos. 5,994,624 and 5,981,274), including microinjection (Harland and Weintraub, 1985; U.S. Pat. No. 5,789,215); by electroporation (U.S. Pat. No. 5,384,253); by calcium phosphate precipitation (Graham and Van Der Eb, 1973; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Kato et al., 1991); by microprojectile bombardment (WO 94/09699; U.S. Pat. No. 5,610,042); by agitation with silicon carbide fibres (Kaeppler et al., 1990; U.S. Pat. No. 5,302,523); by Agrobacterium-mediated transformation (U.S. Pat. Nos. 5,591,616 and 5,563,055); by PEG-mediated transformation of protoplasts (Omirulleh et al., 1993; U.S. Pat. No. 4,684,611); or by desiccation/inhibition-mediated DNA uptake (Potrykus et al., 1985). Through the application of techniques such as these, an organelle, cell, tissue or organism may be stably or transiently transformed, as desired.

### Transgene Insertions

In various embodiments, additional transgenes may be inserted into the nucleic acid or viral vector. For example, in embodiments, one, two or three transgenes may be inserted into the former locus of a wild-type viral gene that has been deleted. Suitable candidate genes for deletion/replacement may include B8R or particularly TK (also known as J2R). In some strains, in addition to the transgene(s) present at the site of the TK (and/or B8R) deletion, the engineered virus genome may also have at least one transgene inserted into an additional locus on the vaccinia virus genome that is not the locus of the deleted TK (or other) gene. Suitably, the replacements/insertions are located at the locus of genes that are non-essential for vaccinia viral infection and replication. Some such transgenes may beneficially encode cytokines such as GM-CSF; pro-drug converting enzymes such as cytosine deaminase (CD); theranostic payloads such as sodium iodide symporter (NIS); affinity reagents such as bispecific antibodies; and imaging/detection agents such as luciferase, Renilla luciferase-GFP fusion protein, β-galactosidase, β-glucuronidase, or green fluorescent protein.

In various embodiments of the engineered vaccina virus/viral genome of this disclosure, at least one transgene may be inserted at the 5′ and/or 3′ boundaries of a deleted viral gene locus, or towards the 5′ end and/or towards the 3′ end of a truncated endogenous viral gene.

In various, embodiments at least three, four, five or more transgenes are inserted into the modified vaccinia virus genome.

In some particularly suitable embodiments, the oncolytic vaccinia virus into which a fusion/chimeric gene as described herein is introduced is a Copenhagen strain oncolytic vaccinia virus that lacks a functional TK gene and has a transgene that expresses human GM-CSF (e.g. a current clinical example is known as ‘Pexa-vec’). Accordingly, the present disclosure provides engineered vaccinia virus, particularly engineered Copenhagen strain vaccinia virus, that may have one or more beneficial effect/demonstrable improvement over a prior art clinical example, such as greater ability to avoid a host immune system and, hence, greater infectivity and/or oncolytic activity.

### Virus Propagation

The present invention relates to vaccinia viruses, including those constructed with one or more gene deletions compared to the corresponding wild-type genome, such that the virus exhibits desirable properties; particularly for use against cancer cells, i.e. so-called ‘oncolytic viruses’, while being less toxic or non-toxic to non-cancerous cells.

By way of example, a number of different protocols are known to the skilled person which can be used to produce recombinant vaccinia viruses as described herein, and all such methods are envisaged for use in the context of the present disclosure.

First, any suitable molecular biology method can be employed for generating mutated/engineered viruses through the use of recombinant DNA technology. For example, vaccinia virus may be propagated using the methods described by Earl and Moss in Ausubel et al., 1994 or the methods described in WO2013/022764.

For example, to generate mutations in the vaccinia virus genome, native and modified polypeptides may be encoded by a nucleic acid molecule comprised in a vector. Vectors include plasmids, cosmids, viruses (bacteriophage, animal viruses, and plant viruses), and artificial chromosomes (e.g., YACs). The person skilled in the art would be well equipped to construct a vector through standard recombinant techniques, such as are described in Sambrook et al., (1989) and Ausubel et al., 1994, both incorporated herein by reference. In addition to encoding a modified polypeptide, a vector may encode non-modified polypeptide sequences such as a tag or targeting molecule. In order to propagate a vector in a host cell, it suitably contains one or more origins of replication sites (often termed ‘ori’), which are specific nucleic acid sequences at which polynucleotide replication is initiated. Alternatively, an autonomously replicating sequence (ARS) can be employed if the host cell is yeast.

In the context of expressing a heterologous nucleic acid sequence, the term ‘host cell’ refers to a prokaryotic or eukaryotic cell, and it includes any transformable organisms that is capable of replicating a vector and/or expressing a heterologous gene encoded by a vector. A host cell can be used as a recipient for vectors or viruses.

Host cells may be ‘transfected’ or ‘transformed’, which refers to a process by which exogenous nucleic acid, such as an engineered gene, vector or viral genome, is transferred or introduced into the host cell. A transformed cell includes the primary subject cell and its progeny. Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained, for example, through the American Type Culture Collection (ATCC; www.atcc.org). An appropriate host can be determined by the skilled person based on the vector backbone and the desired result. A plasmid or cosmid, for example, can be introduced into a prokaryote host cell for replication of many vectors.

Many host cells from various cell types and organisms are available and would be known to the person skilled in the art. Similarly, a viral vector may be used in conjunction with either a eukaryotic or prokaryotic host cell, particularly one that is permissive for replication or expression of the vector. Some vectors may employ control sequences that allow it to be replicated and/or expressed in both prokaryotic and eukaryotic cells. The skilled person would further understand the conditions under which to incubate any suitable host cells to maintain them and to permit replication of a vector. Also understood and known are techniques and conditions that would allow large-scale production of vectors, as well as production of the nucleic acids encoded by vectors and their cognate polypeptides, proteins, or peptides.

The engineered vaccinia viruses of this disclosure can be produced by methods known to the person skilled in the art. In certain embodiments, the modified oncolytic virus can be propagated in suitable host cells, e.g. selected from HeLa cells, 293 cells, or Vero cells. Once expressed and released from the host cells, engineered virus virions may be isolated from host cells and stored in conditions that promote stability and integrity of the virus, such that loss of infectivity over time is minimised. In certain exemplary methods, the modified oncolytic viruses are propagated in host cells using cell stacks, roller bottles, or perfusion bioreactors. In some examples, downstream methods for purification of the modified oncolytic viruses can comprise filtration (e.g. depth filtration, tangential flow filtration, or a combination thereof), ultracentrifugation, or chromatographic capture.

The engineered vaccinia virus so obtained may then be stored, e.g. by freezing or drying, such as by lyophilisation. In certain embodiments, prior to administration to a subject or in vitro cells or cell culture medium, the stored engineered vaccinia virus can be reconstituted (if dried for storage) and diluted in a pharmaceutically acceptable carrier for administration.

### Therapeutic Peptides and Nucleic Acids

The ‘active agent’ or therapeutic molecule may comprise any suitable engineered vaccinia virus particle as described herein, or any polypeptide moiety that is expressed by an engineered viral genome according to this disclosure—such a polypeptide (moiety) may include polypeptides that form structural components of an engineered vaccinia virus particle or polypeptides that are expressed from the engineered vaccinia virus genome as free polypeptides, for example, that are released from the virus particle or host cell. Particularly beneficial polypeptides include those that elicit an immune response, such as antigens or cytokines.

The invention also encompasses nucleic acid molecules that encode the peptide sequences of the invention. In view of codon redundancy, it will be appreciated that many slightly different nucleic acid sequences may accurately code for each of the fusion proteins of the invention, and each of these variants is encompassed within the scope of the present invention. The skilled person can readily determine suitable nucleic acid sequences for encoding each of the fusion proteins of the invention, and may select appropriate codon codes according to the system in which fusion protein is to be expressed (e.g. mouse, rabbit or human). Any nucleic acid sequences that encode for the peptides of the invention are encompassed within the invention.

Taking into account that minor modifications to the primary sequence of the peptides/proteins of the invention can be made without substantially altering the scope of the claimed invention, the invention should be considered to encompass, in addition, any polypeptide sequences that are substantially the same as the specific amino acid sequences disclosed herein. For example, the claimed invention encompasses polypeptide sequences that have at least 80% identity to the SEQ ID NOs of the polypeptide sequences disclosed herein; at least 85% identity, at least 90% identity, at least 95% identity, at least 98% identity, at least 99% identity or approx. 100% identity to the polypeptide sequences of the SEQ ID NOs explicitly disclosed herein.

Similarly, the claimed invention encompasses polynucleotide sequences that—particularly having regard to the effect of codon redundancy—have at least 70% identity to the polynucleotide SEQ ID NOs disclosed herein; at least 80% identity, at least 85% identity, at least 90% identity, at least 95% identity, at least 98% identity, at least 99% identity or approx. 100% identity to the polynucleotide sequences encoding the SEQ ID NOs explicitly disclosed herein.

Conveniently, the engineered vaccinia viral genomes according to the invention comprise at least one modified gene encoding a viral coat protein. In particular, the modified viral gene may be a fusion or chimeric gene comprising a polynucleotide sequence encoding a viral envelope protein and a heterologous polynucleotide sequence encoding a complement regulatory protein. As such, the heterologous polynucleotide is linked to the viral coat protein by a polynucleotide sequence encoding a covalent polypeptide linker sequence. Any suitable polypeptide linker sequence may be used; for example, suitable linker sequences may have from about 4 to about 50 amino acids, such as from about 5 to about 40 amino acids or from about 8 to about 30 amino acids, from about 10 to about 25 amino acids, or from about 12 to about 20 amino acids.

Non-limiting examples of polypeptide linker sequences for joining the respective portions/halves of the fusion proteins of this disclosure include linkers based predominantly on Gly and Ser residues; such as based on respective Gly₃Ser or Gly₄Ser sequences. Particularly suitable linker sequences include (Gly₄Ser), (Gly₄Ser)₂ and (Gly₄Ser)₃ (SEQ ID NOs: 34, 35 and 36, respectively)

### Nucleic Acids and Peptide Expression

The nucleic acid molecules according to the invention and, where appropriate, the engineered viruses of the invention may be produced by recombinant DNA technology and standard viral expression and purification procedures. Thus, the invention further provides nucleic acid molecules that encode the engineered viral coat proteins and/or heterologous (non-viral) proteins of the invention as well as their derivatives; and nucleic acid constructs, such as expression vectors that comprise nucleic acid encoding peptides and derivatives according to the invention. Typically, in accordance with this disclosure, nucleic acid constructs of the disclosure are incorporated into a suitable vaccinia virus vector for expression of engineered polypeptides in concert with production of engineered vaccinia virus particles. However, in some cases expression of engineered polypeptides of this disclosure may be desirable from non-viral expression sources.

For instance, the DNA encoding the relevant polypeptides can be inserted into a suitable expression vector (e.g. pGEM®, Promega Corp., USA), where it is operably linked to appropriate expression sequences, and transformed into a suitable host cell for protein expression according to conventional techniques (Sambrook J. et al., Molecular Cloning: a Laboratory Manual, Cold Spring Harbor Press, Cold Spring Harbor, NY). Suitable host cells are those that can be grown in culture and are amenable to transformation with exogenous DNA, including bacteria, fungal cells and cells of higher eukaryotic origin, preferably mammalian cells (e.g. particularly mice or human).

The term ‘operably linked’, when applied to nucleic acid sequences of this disclosure, for example in an expression vector or construct, indicates that the sequences are arranged so that they function cooperatively in order to achieve their intended purposes, i.e. a promoter sequence allows for initiation of transcription that proceeds through a linked coding sequence as far as the termination sequence.

It will be appreciated that, depending on the application, the fusion or other heterologous proteins of the invention may comprise an additional peptide sequence or sequences at the N- and/or C-terminus for ease of protein expression, cloning, and/or peptide or RNA stability, without changing the sequence of the final polypeptide sequence, such as the intended viral coat fusion peptide of the invention. Where considered appropriate, suitable N-terminal leader peptide sequences for incorporation into peptides of the invention are known to the person skilled in the art from the available literature.

In some applications it may be desirable to control the expression of the fusion or other heterologous polypeptides of the invention by use of inducible promoters or by use of modified wild type/native viral promoter sequences. In this way, for example, it may be possible to desirably alter heterologous/fusion protein expression patterns, e.g. to occur at different stages of viral morphogenesis. In some embodiments, native promoter sequences that are operably associated with one or more of the heterologous or fusion protein encoding nucleic acid sequences are modified to improve or reduce expression, and particularly, to cause expression of the gene product earlier or later in viral morphogenesis than the wild-type promoter sequence on which it is based.

It is particularly desirable to express the chimeric or fusion peptides of the invention, such as engineered viral coat proteins, from vectors—particularly viral vectors—suitable for use in vivo or ex vivo, e.g. for therapeutic applications (gene therapy). Where the invention relates to a therapy that involves use of engineered nucleic acid constructs for expression of protein in vivo, the expression system selected should be capable of expressing protein in the appropriate tissue/cells where the therapy is to take effect. Desirably an expression system for use in accordance with the invention is also capable of targeting the nucleic acid constructs or peptides of the invention to the appropriate region, tissue or cells of the body in which viral assembly and/or the eventual therapeutic treatment is intended.

Suitable medical uses and methods of therapy may, in accordance with the disclosure, encompass the combined use—either separate, sequential or simultaneous—of more than one different engineered viral vector. In other embodiments, the disclosure encompasses the use of one or more engineered viral vector as described herein, in combination with one or more additional therapeutic agent.

As the person skilled in the art would understand, strict compliance to the sequences provided is not necessary for the function of any promoter, provided that functional elements, e.g. enhancers, and their spatial relationships are essentially maintained. In particular, the promoter sequences provided herein may comprise flanking restriction sites for cloning into a vector.

Where appropriate, the person skilled in the art would know to adapt these restriction sites to the particular cloning system used, as well as to make any point mutations that may be required in the sequence of the promoter to remove e.g. a cryptic restriction site.

Suitable inducible systems may use small molecule induction, such as the tetracycline-controlled systems (tet-on and tet-off), the radiation-inducible early growth response gene-1 (EGR1) promoter, and any other appropriate inducible system known in the art.

### Diseases and Disorders

The agents, compositions, methods and uses of the present invention may be particularly suitable for the treatment of a wide range of diseases and disorders, including, for example, any disease or disorder that would benefit from a targeted reduction in the number of cells associated with the disease phenotype or disease progression. In particular, diseases and disorders that may be treated in accordance with the invention include cancers and/or proliferative or oncologic diseases, such as cancers of hematopoietic origin, or solid tumors.

The skilled person will appreciate that proliferative diseases may be associated with: (1) pathological proliferation of normally quiescent or normally proliferating cells; (2) pathological migration of cells from their normal location (e.g., metastasis of neoplastic cells); (3) pathological expression of proteolytic enzymes such as the matrix metalloproteinases (e.g., collagenases, gelatinases, and elastases), which can lead to unwanted turnover of cellular matrices; and/or (4) pathological angiogenesis, as occurs in proliferative retinopathy and tumor metastasis. Exemplary proliferative diseases include cancers, benign neoplasms, and angiogenesis that accompanies and facilitates a disease state (defined above as pathologic angiogenesis).

The compositions, agents, methods and uses of the present invention may have beneficial effects in treating a wide range of proliferative diseases and disorders and/or reducing the symptoms thereof; for example, by preventing cellular proliferation and especially in promoting cell death of pathogenic cells.

The invention may have utility in multiple cancer types, and/or have beneficial effects on tumour progression (such as, for example, reversing tumour progression) in vivo and/or in vitro. In particular, the invention may be useful in the treatment of lung cancers (in particular lung adenocarcinomas), cervical cancer, breast cancer, cardiac cancer, colon cancer, prostrate cancer, brain glioblastoma, pancreatic cancer, leukemia (e.g. acute monocytic leukemia), lymphoma, kidney cancer, colorectal cancer, bladder cancer, testicular cancer, gastrointestinal cancer, liver cancer (e.g. hepatocarcinoma), and/or glioblastoma. The invention may also be useful in the treatment of one or more of skin cancer (e.g. melanoma), head and/or neck cancer, gallbladder cancer, uterine cancer, stomach cancer, tyroid cancer, laryngeal cancer, lip and/or oral cancer, throat cancer, ocular cancer and bone cancer.

In some particular embodiments, the cancer may be selected from any one or more of the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

Furthermore, this disclosure also encompasses the therapeutic use of therapeutic agents and compositions of the invention, and methods for inhibiting or preventing local invasiveness or metastasis, or both, of any type of primary cancer. For example, the primary cancer can be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer can be liver cancer. For example, the liver cancer can be Hepatocellular carcinoma (HCC) and/or metastases to the liver.

Moreover, this disclosure can be used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer can be halted, disrupted, or delayed by the uses and methods of this disclosure involving the therapeutic agents disclosed herein.

As such, the invention provides agents and compositions for use in medicine and, in particular, for use in the treatment of cancers selected from lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, tyroid cancer, bone cancer and liver cancer. Methods for the treatment of such diseases are also provided. The uses and methods may comprise administering the agents according to the invention to a patient in need thereof.

The compositions, agents, methods and uses of the present invention may provide benefits in the treatment of any or all such diseases and disorders.

### Therapeutic Compositions

A fusion peptide, nucleic acid or engineered virus particle (e.g. ‘therapeutic agents’) of the invention may be incorporated into a pharmaceutical composition for use in treating an animal; preferably a human. A therapeutic peptide, nucleic acid or engineered virus of the invention (or derivative thereof) may be used to treat one or more diseases or infections, depending on the activity/properties of the engineered nucleic acid construct, fusion peptide, or virus. In various embodiments, a nucleic acid encoding the therapeutic peptide may be inserted into an expression construct/vector—particularly a vaccinia virus vector—and incorporated into pharmaceutical formulations/medicaments for the same purpose.

As will be understood by the person of skill in the art, potential therapeutic agents, such as according to this disclosure, may be tested in an animal model, such as a rabbit or mouse, before they can be approved for use in human subjects. Accordingly, fusion proteins or viruses of the invention may be expressed in vivo in rabbit or mice or ex vivo in rabbit or mouse cells as well as in humans/human cells. In accordance with the invention, appropriate expression cassettes and expression constructs/vectors may be designed for each animal system specifically. As such, the engineered fusion protein of the disclosure may be modified according to known or identified abilities to inhibit the complement pathway in the chosen animal host.

The therapeutic peptides and nucleic acids of the invention may be particularly suitable for the treatment of diseases, conditions and/or infections that can be targeted (and treated) intracellularly, for example, by targeting of vaccinia virus to an animal cell; and may also be suitable for in vitro and ex vivo applications. As used herein, the terms ‘therapeutic agent’ and ‘active agent’ encompass peptides and nucleic acids that encode a peptide of the invention, and also viral particles comprising peptides and/or nucleic acids as described herein. Therapeutic nucleic acids of the invention encompass modified/engineered vaccinia virus genomes, which comprise nucleic acid sequences encoding viral coat fusion proteins according to this disclosure and optionally one or more additional heterologous gene and/or protein.

Therapeutic uses and applications for the therapeutic agents of the invention include any disease, disorder or other medical condition that may be treatable by expressing an engineered vaccinia virus in the subject to be treated.

In accordance with aspects and embodiments of the present invention, particularly preferred diseases include cancers and other proliferative diseases or disorders, as disclosed elsewhere herein.

One or more additional pharmaceutically acceptable ‘carrier’ (such as diluents, adjuvants, excipients or vehicles) may be combined with the therapeutic agent(s) of the invention in a pharmaceutical composition. Suitable pharmaceutical carriers are described in “Remington's Pharmaceutical Sciences” by E. W. Martin. Pharmaceutical formulations and compositions of the invention are formulated to conform to regulatory standards and can be administered orally, intravenously, topically, or via other standard routes. As used herein, the term ‘carrier’ includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase ‘pharmaceutically acceptable’ or ‘pharmacologically-acceptable’ refers to molecular entities and compositions that do not produce an allergic or similar untoward reaction when administered to a human.

In accordance with the invention, the therapeutic agent(s) may be manufactured into medicaments or may be formulated into pharmaceutical compositions. When administered to a subject, a therapeutic agent is suitably administered as a component of a composition that comprises a pharmaceutically acceptable vehicle. The molecules, compounds and compositions of the invention may be administered by any convenient route, for example, intradermal, intramuscular, intraperitoneal, intravenous, subcutaneous, intranasal, epidural, oral, sublingual, intranasal, intravaginal, transdermal, rectally, by inhalation, or topically to the skin. Administration can be systemic or local. Delivery systems that are known also include, for example, encapsulation in microgels, liposomes, microparticles, microcapsules, capsules, etc., and any of these may be used in some embodiments to administer the agents of the invention. Any other suitable delivery systems known in the art are also envisaged in use of the present invention.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle particularly when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

In some embodiments the therapeutic compositions or medicaments of the invention are formulated in accordance with routine procedures as a pharmaceutical composition adapted for oral administration (more suitably for human beings). Compositions for oral delivery may be in the form of tablets, lozenges, aqueous or oily suspensions, granules, powders, emulsions, capsules, syrups, or elixirs, for example. Thus, in various embodiments, the pharmaceutically acceptable vehicle may be a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. The person skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the therapeutic agent(s) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the peptide or nucleic acid or derivative either alone or as a mixture in different ratios.

Typically, compositions for intravenous administration comprise sterile isotonic aqueous buffer. Where necessary, the compositions may also include a solubilising agent.

Mixtures of the viral particles or nucleic acids described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form may be sterile and may be sufficiently fluid to enable injection by an appropriate syringe. Suitably, the composition is stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art.

Another suitable route of administration for the therapeutic compositions of the invention is via pulmonary or nasal delivery.

Additives may be included to enhance cellular uptake of a therapeutic agent of the invention, such as the fatty acids, oleic acid, linoleic acid and linolenic acid.

The therapeutic agents of the invention may, in some embodiments, also be formulated into compositions for topical application to the skin of a subject.

In embodiments of the invention the therapeutic compositions may include only one therapeutic agent of the invention; or may include two or more e.g. two complementary therapeutic agents of the invention. For example, inhibition of different proteins involved in the complement pathways of the target animal may be achieved by more than one engineered protein of the disclosure. In particular, an engineered virus according to this disclosure may express two chimeric/fusion proteins, each comprising a heterologous protein or protein domain for targeting different stages of or different complement pathways, fused to expressed vaccinia proteins—such as two different viral envelope/structural proteins. When two (or more) therapeutic agents are contemplated, the different peptides or encoding nucleic acid constructs such as viral vectors or viral particles may be incorporated into the same pharmaceutical composition, or may be manufactured separately. Where two (or more) pharmaceutical compositions are manufactured for administration to the same individual, it will be appreciated that the compositions may be administered simultaneously, sequentially, or separately, as directed/required.

### Methods for Delivery of Engineered Vaccinia Virus

Immunisation through vaccinia virus infection is typically extremely safe and results in mild or asymptomatic infection in healthy individuals. Complications or adverse effects are generally only seen in vulnerable individuals, such as the immunocompromised. As a vaccine, the virus is typically administered by a multiple skin puncture technique using a bifurcated needle. In order to efficiently deliver virus to solid tumours, however, different routes of administration are required.

Any suitable delivery method for administering the therapeutic agents, engineered vaccinia virus or therapeutic compositions of the invention may be used in accordance with the disclosure. A suitable route of administration may be determined by the skilled medical practitioner, and may be dependent on one or more factors.

Delivery of the therapeutic agents or compositions of the invention can be systemic or local. For example, the route of administration may be determined by the location and/or nature of the disease, e.g. cancer to be treated, and may include: intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration, for example, as described in U.S. Pat. Nos. 5,543,158; 5,641,515 and 5,399,363. Typically, convenient forms of systemic administration may include oral administration, parenteral administration, intranasal administration, sublingual administration, rectal administration, transdermal administration, or any combinations thereof.

In various embodiments the therapeutic composition may be administered directly to the site of the tumour. In some particularly beneficial embodiments, therapeutic compositions of the disclosure may be delivered by injection into the vasculature of the subject,

In some embodiments, (continuous) administration/administration over a prolonged period of time may be preferred and may be achieved by any suitable mechanism, for example, by implanting a catheter into a tumor or into tumor vasculature. Suitably, the dose of the therapeutic composition via continuous perfusion may be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Injection of nucleic acid constructs may be delivered by syringe or any other method used for injection of a solution, as long as the expression construct (e.g. virus) can pass through the particular gauge of needle required for injection. In some embodiments, a needleless injection system may be used, as known to the skilled person, e.g. as described in U.S. Pat. No. 5,846,233.

### Therapeutic Treatment Regimes and Doses

Treatment regimens may vary, and often depend on the type and/or location of the tumour, the stage/progression of the disease, and/or the health and age of the patient. For example, some tumours may respond better to treatment with localised and/or high concentration doses of the therapeutic composition, whereas other diseases (and individuals) may benefit from a more diffuse and/or low dose and/or prolonged administration of the therapeutic agent. The skilled clinician will be able to determine a suitable therapeutic treatment regime under each circumstance.

In various aspects and embodiments, the disclosure provides therapeutic uses and methods for treating a subject by administration of one or more engineered vaccinia viruses, as disclosed herein. By contacting a target cell or first population of target cells with an engineered vaccinia virus, the virus may infect the target cell/first population of target cells and reproduce in those target cells thereby causing a toxic effect sufficient to kill the target cell. Accordingly, the disclosure provides uses and methods for reducing tumour size and/or growth by killing target infected cells via a toxic effect caused by the engineered virus in the target cancer cell(s). The uses and methods comprise administering to an individual, or to a cancer cell or first population of target cells of the individual, a therapeutically effective amount of an engineered vaccinia virus of this disclosure, or—as described above—a pharmaceutical composition containing an engineered vaccinia virus or other therapeutic agent of the invention. According to the therapeutic methods of the invention, upon infection of a first target cell (or first population of target cells), the vaccinia virus may reproduce within the first (population of) target cells and on release of new virus from the first target cell (or first population of target cells), a second target cell or second population of target cells may be infected with the released virus. Beneficially, therefore, by use of such an engineered virus, it is not necessary to directly administer to or infect every target cell in a tumour or subject with an initial population of engineered vaccinia virus. Rather, by enabling the engineered virus to maintain its proliferative capacity within a target cell, but to avoid rapid destruction by the host (individual) immune system, it is possible to infect and thereby destroy a far larger population of target cancer cells than would otherwise be possible through an initial, single administration of the therapeutic agent. Moreover, through the use of the vaccinia virus's natural ability to invade and infect neighbouring or remote target cells, the uses and methods of the invention may enable the destruction of cancer cells that it would not otherwise be easy to or even possible to administer the therapeutic agents to directly.

Accordingly, this disclosure further provides a method of treating a cancer or tumour, or for inhibiting the growth and/or proliferation of a second cancer cell comprising administering, to a first cancer cell, an engineered vaccinia virus according to the disclosure such that the first cancer cell is infected with the virus and subsequently, the second cancer cell is infected with the virus. The second cancer cell may be remote from the first cancer cell; for example, the second cancer cell may be in a solid tumour remote from a solid tumour in which the first cancer cell is located; or the second cancer cell may be a metastasis of the first cancer cell.

An effective amount of an engineered vaccinia virus of the present disclosure or a pharmaceutical composition thereof, can include an amount sufficient to induce oncolysis/killing of a target cancer cell and/or the disruption of lysis or proliferation of a target cancer cell, or the inhibition or reduction in the growth or size of a target cancer cell or tumour. Reducing the growth of a tumour or target cancer cell may be manifested, for example, by cell death or a slower replication rate or reduced growth rate of a tumour comprising the cell, or a prolonged survival of a subject containing the cancer cell.

Accordingly, in some embodiments, there is provided therapeutic uses and methods of treating a subject having a cancer or a tumor comprising administering, to the subject, an effective amount of an engineered vaccinia virus of the disclosure. An effective amount in such uses and methods can include an amount that reduces growth rate or spread of the cancer or that prolongs survival in the subject. In some embodiments, the tumour may be entirely or substantially eradicated. The uses and methods of the disclosure may include administering to the subject or individual the engineered vaccinia virus. Administration may be by any suitable means, for example, by injection or ingestion. Administration may be systemic or local, e.g. directly to the site of a tumour. In one embodiment, systemic delivery is preferred.

The therapeutic compositions and/or therapies may include various ‘unit doses’, where a unit dose can be defined as containing a predetermined quantity of the therapeutic agent or composition. Identifying a suitable quantity to be administered, and the particular route of administration and formulation, are within the skill of those in the clinical arts. A unit dose may be administered as a single injection or tablet, for example; but may also be administered over a prolonged period of time, such as via a continuous infusion over a set duration.

In relation to embodiments comprising engineered vaccinia virus, a unit dose may conveniently be described in terms of plaque forming units (pfu) for the viral construct, rather than by weight or molar concentration. A unit dose may be described alternatively as viral particles, infectious virus (PFU) or Tissue Culture Inhibitor Dose 50% (TCID50). Unit doses may range from between 1×10³ and 1×10¹⁵ PFUs; for example, about 1×10⁴, about 1×10⁶, about 1×10⁸, about 1×10¹⁰, about 1×10¹² or about 1×10¹⁴ or higher, per mL, per kg, per dosage form (e.g. per tablet or per injection). Deliver of such dosages may be directly to the tumor or tumor site, or delivery may be systemic.

In certain embodiments, the modified oncolytic virus can be administered in one or more doses over a set period of time (for example, over a 12 or 24 hour period), which together make a ‘unit dose’. In other embodiments each individual administration may be considered a ‘unit dose’.

Although the engineered vaccinia virus according to this disclosure may remain viable in vivo for longer than a comparison vaccinia virus absent the modifications described herein, the therapeutic compositions and/or agents of this disclosure may be administered in multiple doses (e.g., 2, 3, 4, 5, 6 or more doses), as deemed appropriate, within a suitable treatment period—for example, over a period of about 1, 2, 3, 4, 5, 6, 7, 8, 9, 10, 11 or 12 days, weeks, months or years as deemed appropriate. The frequency of administration of the engineered vaccinia virus or the pharmaceutical compositions as described herein may be determined by the skilled practitioner, but may be, for example, once daily, twice daily, once every week, once every 2 weeks, once a month, once every 2 months, once every 3 months, once every 6 months and so on.

Further, in accordance with the therapeutic treatment regimes that may be devised, the unit dose may vary according to the stage or type of treatments, as well as having regard to the size and/or age of the subject, or the type of cancer or tumour that it is intended to treat. For example, in some embodiments, the first dose of the engineered vaccinia virus, therapeutic agent or pharmaceutical composition of this disclosure administered to the subject may be less than or higher than any second, third or subsequent dose that may be administered. In other embodiments, during a first administration period of time the dose may be lower or higher than the dose administered during a second, third or subsequent administration period. The skilled practitioner can determine the duration of any such administration periods, for example, about 1 day, about 1 week or about 1 month, or for any intermediate or longer period of time.

In some examples, the subject to be treated may be administered one or more additional therapeutic agent or may receive one or more additional conventional (such as radiation treatment or chemotherapy) or complementary therapy. For example, in some embodiments the subject may be put on a reduced carbohydrate diet, e.g., a ketogenic diet prior to, concurrent with, and/or following administration of a therapeutic composition as disclosed herein.

### Combination Therapies

In various embodiments, the uses and methods of this disclosure comprise administering a therapeutic agent of this disclosure (e.g. an engineered vaccinia virus or an engineered vaccinia virus vector as disclosed herein), or a pharmaceutical composition containing a therapeutic agent of this disclosure, in combination with one or more additional therapy or therapeutic agent. As disclosed herein, the one or more additional therapy or therapeutic agent agent may be administered simultaneously, sequentially or separately (before or after) the therapeutic agent according to this disclosure.

The additional therapeutic agent may be an engineered vaccinia virus or an engineered vaccinia virus vector according to this disclosure, such that one or two engineered vaccinia viruses and/or one or two virus vectors of the invention are administered in combination. Other examples of the further therapy or therapeutic agent can include, but are not limited to chemotherapy, radiation, oncolytic viral therapy with an additional virus, treatment with immunomodulatory proteins, an anti-cancer agent, or any combination thereof.

In particular embodiments, the additional therapeutic agent is an anti-cancer agent or cancer therapy. Anti-cancer agents can include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. Cancer therapies can include chemotherapy, biological therapy, radiotherapy, immunotherapy, hormone therapy, anti-vascular therapy, cryotherapy, toxin therapy and/or surgery or combinations thereof.

Combination of the modified oncolytic vaccinia virus with chemotherapy beneficially achieves a synergistic effect with the additional therapeutic agent, such that the typical dose of the additional therapeutic agent alone can be reduced. As such, the therapeutic uses and methods of this disclosure may reduce toxicities associated with chemotherapy without reducing the therapeutic benefit of the invention.

In various embodiments, treatment of cancer may involve a surgical procedure. Such surgery can include resection in which all or part of a cancerous tissue is physically removed from the subject (e.g. by excision) and/or otherwise destroyed. Tumor resection refers to physical removal of at least part of a tumor. In addition to tumor resection, treatment by surgery includes laser surgery, cryosurgery, electrosurgery, and microscopically controlled surgery (Mohs' surgery). In some embodiments, it may be determined that a tumour (or part thereof) should be removed by resection, or alternatively, that the tumour is not suitable for resection. In such circumstances, embodiments of the invention may include treating the subject with therapeutic agents or compositions of this disclosure in order to improve the outcome of resection, or to enable the tumour or part thereof to be excised. Therapeutic treatments of the present disclosure may, therefore, increase the resectability of a tumour, e.g. due to shrinkage at tumour margins or by elimination of invasive portions. Additional treatments subsequent to resection will serve to eliminate microscopic residual disease at the tumor site.

The therapeutic agents of the disclosure may particularly be used in combination with (i.e. before and/or after) a surgical treatment of a cancer, for example, for the treatment and/or destruction of tumour margins surrounding the site of the cancerous tissue. In embodiments, upon excision of part of or all of cancerous cells, tissue, or tumor, one or more therapeutic agent of the disclosure, for example, an engineered vaccinia virus may be administered to the site of removal, by perfusion, direct injection or local application to the area, e.g. to the cell walls surrounding the cavity or space left by removal of the tumour. Depending on the treatment regime, such treatment may be repeated, for example, every 1, 2, 3, 4, 5, 6, or 7 days, or every 1, 2, 3, 4, 5 or 6 weeks, or every 1, 2, 3, 4, 5, 6, 7, 8, 9, 10, 11, or 12 months.

### Methods for Determining Efficacy

Aspects and embodiments of the disclosure also encompass methods for determining the efficacy of the therapeutic agents, such as engineered vaccinia viruses, of the disclosure. For example, methods may include determining the infectivity, anti-tumour activity, or amount of tumour specific viral replication of an engineered vaccinia virus of the disclosure. For this purpose, such an engineered vaccinia virus may further comprise a reporter gene construct capable of identifying an engineered virus of the disclosure. Such a reporter gene construct may, for example, express a reporter protein, such as luciferase, Renilla luciferase-GFP fusion protein, β-galactosidase, β-glucuronidase, or green fluorescent protein.

The method may comprise: (i) administering to a subject a therapeutically effective amount of a therapeutic agent—such as an engineered vaccinia virus or a pharmaceutical composition according to the present disclosure, which virus, vector or nucleic acid is capable of further expressing a reporter protein, alone or in combination with a further therapeutic agent; (ii) collecting a first biological sample from the subject immediately after administering the virus, vector or nucleic acid and determining the level of the luciferase reporter in the first biological sample; (iii) collecting a second biological sample from the subject following the administration in step (i) and determining/detecting the level of the reporter in the second biological sample, wherein the therapeutic agent (e.g. engineered vaccinia virus) is determined to be efficacious (e.g. infective, demonstrate anti-tumor activity and/or exhibit tumour specific viral replication) if the level of luciferase is higher in step (iii) than in step (ii). The second biological sample is collected a predetermined time after the first sample has been collected, for example, up to 1 day, up to 1 week or up to 1 month after the first sample is collected. In embodiments, the second sample may be collected about 1 hour, about 2 hours, about 4 hours, about 8 hours, about 12 hours, about 24 hours, about 2 days, about 4 days, about 7 days, about 14 days, about 1 month, or up to about 2 months after the administration in step (i).

In some embodiments, the method of determining the efficacy of the therapeutic agent (e.g. an engineered virus of the disclosure) may further comprise, detecting in steps (ii) and (iii), the level of one or more cytokine. The cytokine may be selected from one more of IL-2, IL-7, IL-8, IL-10, IFN-γ, GM-CSF, T F-a, IL-6, IL-4, IL-5, and IL-13.

Methods according to these aspects and embodiments may further comprise comparing the efficacy of a therapeutic agent (e.g. an engineered vaccinia virus) according to this disclosure with a comparison therapeutic agent (e.g. vaccinia virus), which is identical to the engineered therapeutic agent (e.g. vaccinia virus) of this disclosure, including a reporter construct, except that it is lacking the fusion protein and optional additional heterologous gene(s) expression constructs described herein. Suitably, the reporter construct of the comparison or control vaccinia virus expresses a different reporter to that of the engineered therapeutic agent (e.g. vaccinia virus) according to this disclosure. Thus, in some embodiments, the increase in reporter gene expression between steps (ii) and (iii) is compared to the difference in reporter gene expression for the comparison or control therapeutic agent (e.g. vaccinia virus).

Accordingly, the method further comprises, a step (iv) administering to a subject (which subject may be the same or different to the subject of step (i) a therapeutically effective amount of a comparison or control therapeutic agent (e.g. vaccinia virus) expressing a reporter construct; (v) collecting a first biological sample from the subject immediately after administering the therapeutic agent (e.g. virus) of step (iv) and determining the level of the reporter in the first biological sample; and (vi) collecting a second biological sample from the subject following the administration in step (iv) and determining/detecting the level of the reporter in the second biological sample; wherein steps (iv) to (vi) may be carried out simultaneously with streps (i) to (iii).

Other exemplary techniques known to the person skilled in the art for detecting and monitoring viral load after administration of the engineered therapeutic agents and vaccinia viruses of the disclosure may alternatively be used. An exemplary alternative technique is real-time quantitative PCR, such that the steps of determining viral load include performing real-time quantitative PCR of the collected biological samples to detect the engineered virus, rather than determining the amount of a reporter construct.

### Kits

In embodiments, this disclosure provides a kit for administering an engineered vaccinia virus, engineered vaccinia virus vector, or nucleic acid as described herein. In certain embodiments, a kit of this disclosure can include an engineered vaccinia virus, an engineered vaccinia virus vector, or nucleic acid or a (pharmaceutical) composition comprising such a therapeutic agent, as described above. In certain embodiments, a kit of this disclosure can further include one or more components such as instructions for use, devices and additional reagents, and components, such as tubes, containers and syringes for performing the methods disclosed above. In various embodiments, a kit of this disclosure can further include one or more active agent, e.g., at least one selected from the group consisting of an anti-cancer agent, an immunomodulatory agent, or any combinations thereof, that may be administered in combination (separately, sequentially or simultaneously) with the engineered virus.

In some embodiments, a kit according to the disclosure may comprise one or more containers containing an engineered virus and/or an engineered virus vector and/or a nucleic acid or protein according to the invention, one or more additional active agent and/or any reagents as described herein.

In some embodiments, a kit may further include an apparatus or device for administering a therapeutic agent (such as an engineered vaccinia virus) of the disclosure, and/or any additional active agent to a subject. A suitable apparatus or device may include one or more of a hypodermic needle, an intravenous needle, a catheter, a needle-less injection device, an inhaler and/or a liquid dispenser.

Instructions for use of the kit may suitably include a description of what the kit should include and how it should be properly used; for example, how the various components of the kit should be administered to an individual, including timing, concentrations and quantities; proper administration methods and how/whether the individual should be monitored during use/treatment.

### Host Organism Toxicity and Immunogenicity

It was proposed that toxicity and immunogenicity (immunotoxicity) of heterologous peptides when expressed in host organisms might be reduced by optimising the primary peptide sequence to match the primary peptide sequence of natural host peptides.

Polypeptides, including fusion/chimeric proteins according to the invention are up to 20%, 19%, 18%, 17%, 16%, 15%, 14%, 13%, 12%, 11% or 10% non-identical to endogenous/natural peptide sequences found in the host organism; for example, the peptides of the invention may be between approximately 1% and 25%, between approximately 3% and 20% or between approximately 5% and 15% non-identical to an endogenous peptide sequence of the host organism. The host organism may be considered to be a virus, or an animal subject to which the polypeptide will be administered or in which the polypeptide will be expressed.

Sequence identity can be assessed in any way known to the person of skill in the art, such as using the algorithm described by Lipman & Pearson (1985), Science 227, pp 1435; or by sequence alignment.

As used herein, ‘percent identity’ means that, when aligned, that percentage of amino acid residues (or bases in the context of nucleic acid sequences) are the same when comparing the two sequences. Amino acid sequences are not identical, where an amino acid is substituted, deleted, or added compared to the reference sequence. In the context of the present invention, since the subject proteins may be considered to be modular, i.e. comprising several different domains or effector and auxiliary sequences (such as NLS sequences, peptide expression sequences, and viral coat protein sequences), including that the polypeptides may be fusion/chimeric proteins, sequence identity may conveniently be assessed separately for each domain/module of the peptide relative to any homologous endogenous or natural peptide domain/module known in the host organism. This is considered to be an acceptable approach since relatively short peptide fragments (epitopes) of any host-expressed peptides may be responsible for determining immunogenicity through recognition or otherwise of self/non-self peptides when expressed in a host organism in vivo. By way of example, a peptide sequence of 100 amino acids comprising a first host protein domain directly fused to a second host protein domain, wherein neither protein domain sequence has been modified by mutation (but wherein the fusion protein is not a natural protein) would be considered to be 100% identical to host peptide sequences. It does not matter for this assessment whether such separate, fused domain(s) only represent a fragment of a natural, larger protein sequence expressed in the host, or that the fusion itself is not naturally occurring. If one of 100 amino acids has been modified from the natural sequence, however, the modified sequence would be considered 99% identical to natural protein sequences of the host.

Thus, the degree of sequence identity between a query sequence and a reference sequence may be determined by any means available to the person skilled in the art; for example, by sequence alignment.

Where a peptide sequence has been designed/engineered to match (or best match) an endogenous host sequence, such a that of a mouse or human, it may be considered ‘mousified’ or ‘humanised’. In practice, ‘humanisation’ or e.g. ‘mousification’, has the intention of reducing the potential for foreign epitopes in the peptide sequences of the invention. Similarly, the peptide sequence may of course be adapted for any other animal expression system, such as rabbit. Any such deliberate sequence changes should desirably be carried out within the constraints of achieving a fully active (or at least suitably viable) viral coat protein that may be used in the assembly of a viral particle for presenting the fused/chimeric protein at the surface of the vaccinia virus.

In order to improve sequence identity, a suitably ‘host-matched’ fused/chimeric protein sequence for use in humans, may be replaced by a suitable mouse analogue protein for mouse studies. Similarly, to further improve host optimisation, any associated peptide sequences, are preferably selected from human and mouse sequences for expression in humans or mice, respectively.

### EXAMPLES

The invention will now be further illustrated by way of the following non-limiting examples.

Unless otherwise indicated, commercially available reagents and standard techniques in molecular biological and biochemistry were used.

### Materials and Apparatus

The following procedures used by the Applicant are described in Sambrook, J. et al., 1989 supra.: analysis of restriction enzyme digestion products on agarose gels and preparation of phosphate buffered saline. General purpose reagents, oligonucleotides, chemicals and solvents were purchased from Merck Life Science Ltd (UK). Enzymes and polymerases were obtained from New England Biolabs (NEB Inc.; UK).

pUC57-Amp A27L fusions and pUC57-Amp A13L fusions, as in Table 1 (GENEWIZ). BSC-1 cells (ATCC, cat. #CCL-26), HeLa cells (ATCC, cat. #CCL-2). VACV-COP ΔJ2R(TK) Vaccinia virus stock. Lipofectamine 2000 Transfection Reagent (ThermoFisher, cat. #1603027). DMEM media (Gibco™ 41965039), FBS (Gibco™ 10500064), DPBS (Gibco™ 20012027). G418 (Gibco™ 11811031), Sonicating waterbath, 6-well tissue culture plates (Corning™ 3516), 6-cm dishes (Thermo Scientific™ 150288), 15 ml polypropylene tubes (Falcon™ 352196), disposable scraper, Dounce homogeniser, sterile 2 ml microcentrifuge tubes.

### Methods

Cell Preparation and Infection with Wild-Type Vaccinia Virus

BSC-40 cells (2×10⁵ cells/well) (RRID:CVCL_3656), a continuous line of African green monkey kidney cells derived from BSC-1 cells (Hruby, et al., 1979) were seeded in wells of a 6-well tissue culture plate in complete DMEM medium and incubated to 50-80% confluency (37° C., 5% CO₂ overnight). An aliquot of up to 100 μl of the parental virus (Copenhagen strain) was thawed and sonicated in a sonicating waterbath for 1 minute to disperse virus aggregates. Virus was diluted in complete DMEM to 1.2×10⁴ pfu/ml. Medium was removed from confluent monolayers of cells and cells were infected with 0.5 ml diluted vaccinia virus (0.03 pfu/cell) and incubated for 3 hours at 37° C. Infected monolayers were mixed every 15 minutes. After 3 hours the inoculum was washed once with and then replaced with 2 ml complete DMEM (DMEM, 10% FBS, 1% DMEM with non-essential amino acids, 1% sodium pyruvate, 1% Glutamax).

Transfection with Gene Targeting Plasmid

For each well to be transfected, 250 μl serum-free medium was added into two sterile tubes. Six μl Lipofectamine 2000 was added drop-wise directly to the serum-free medium in Tube A, and mixed thoroughly by inversion followed by incubation for 5 minutes. Three μg of DNA was added to a second tube (Tube B) containing 250 μl serum-free media and the contents of Tube B were mixed with the contents of Tube A by dropwise addition of Tube B to Tube A followed by room temperature incubation for 15 minutes. The entire volume of Lipofectamine 2000/DNA mixture was then added drop-wise to the infected cells 3 hours post infection in complete DMEM medium. The dish was gently rocked to ensure even distribution. The transfection mixture was removed after at least 12 hours incubation and replaced with complete DMEM medium containing 2.8 mg/ml G418 followed by incubation for 24 to 48 hours at 37° C. (5% CO₂). After 48 to 72 hours, the cells were dislodged from the wells using a cell scraper and transferred to a 2 ml screw-top sterile microcentrifuge tube. The cell suspension was then lysed by performing three freeze-thaw cycles, each time by freezing at −80° C., thawing in a 37° C. water bath, and vortexing. The cell lysate was stored at −80° C. until needed.

### Plaque Purification

Following integration of the entire gene targeting plasmid into the Vaccinia genome viruses were purified by infection of BSC-40 cells seeded in 6-well dishes. Infected cell lysates were thawed and spun down to sediment cell debris. When the BSC-40 cells were 50-80% confluent wells were infected in duplicate with 1 μl of the infected lysate and two ten-fold dilutions thereof in serum-free media. After one hour the inoculum was replaced with complete DMEM medium containing 2.8 mg/ml G418 followed by incubation for 24 to 48 hrs at 37° C. (5% CO₂). After 48 hours, cells were washed with PBS followed by plaques recovery using a pipet tip. Plaques were dissolved in 100 μl 1 mM Tris pH9 and freeze/thawed three times. Plaque picking was performed three times.

The final viruses were recovered through three rounds of plaque purification as above, or more in case clonal purity was not achieved. Recombination of the targeted genes in the candidate viruses was verified by PCR assay and sequencing.

### Example 1

Introducing Modified Mature Virion (MV) Genes A13L and A27L into the Vaccinia Virus Genome by Homologous Recombination

In order to inhibit complement mediated lysis of virions or opsonisation and uptake by macrophages, complement control proteins were engineered in fusion with vaccinia envelope proteins. The candidate envelope proteins were A13 and A27. As discussed above, whilst A27 protein is localised in the vaccinia envelope in complexes with A17 protein and in a polarised distribution relative to the viral particle, A13 has a non-polar distribution and so is found throughout the envelope.

Each virion envelope gene was modified by introduction of DNA encoding for a fusion protein at either the amino-terminus or carboxy-terminus of the A13 and A27 genes, depending upon which termini is exposed upon the virion surface, while ensuring that the genes of resulting fusion proteins are driven by their native A13L and A27L promoters, respectively. In some embodiments, a V5-tag was also fused to the protein and used to aid in detection of the expressed product. Fusions between the viral genes and their respective fusion partner genes were, in some embodiments, established by insertion of a Gly/Ser linker peptide. Various linkers based on Gly/Ser are envisaged, as described herein; however, it will be appreciated that any suitable peptide linker could alternatively be used, the primary requirement being that the linker sequence allows appropriate folding, assembly and presentation of each protein of the fusion pair at the surface of the virion, and that the linker does not otherwise interfere with the intended function of each protein domain. in some embodiments the linker sequence is optional and so the fusion protein/nucleic acid partners may be fused directly to one another without the use of additional linker sequences.

In the present embodiments, (Gly₄Ser) linkers were used, i.e. a linker peptide based on 4 adjacent Gly residues followed by a Ser residue, which pattern may be repeated one or more times, as desired. In these examples of the invention, a single Gly₄Ser linker peptide was used to fuse the A13 protein sequence to the N-terminus of each respective transgene sequence, and a (Gly₄Ser)₃ linker was used to fuse the A27 protein sequence to the C-terminus of each respective transgene sequence, as described below.

In some exemplified embodiments, a targeting construct encoding the viral gene, A13L or A27L (with or without modifications—i.e. wild type or mutant sequences of each protein), joined to the intended fusion partner by an appropriate linker sequence (where used) and flanked by regions of homology derived from the vaccinia genome of approx. 250 bps were synthesised by Genewiz and cloned into a plasmid (pUC57) containing both a positive mCherry-Neo selection cassette. It will be appreciated, however, that the exact length of the flanking sequence is to an extent arbitrary, and homologous sequences of e.g. 200 bp or more may alternatively be used; the requirement being that the length of homology on each side of the transgene construct is sufficient to direct homologous recombination with a target viral genome.

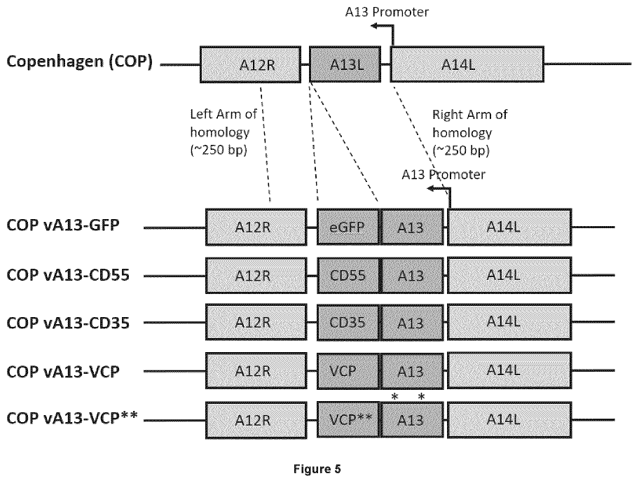

In some embodiments, the flanking sequence for homologous recombination with the appropriate vaccinia virus genome corresponds to (i.e. is homologous with) a sequence region flanking the natural locus of the respective A13L or A27L viral gene, as appropriate, for insertion of the engineered fusion construct in place of the corresponding wild-type gene (see e.g. FIG. 5). In these examples, the resultant modified vaccinia virus does not contain a wild-type A13L and/or A27L gene (depending on the variant strain produced (see below), and so all of the A13 and/or A27 protein produced in the viral envelope will be the engineered fusion protein.

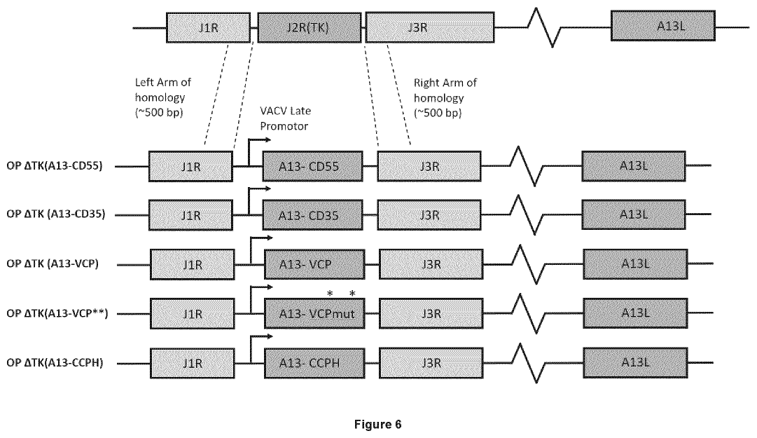

In some applications, it may be desirable that the engineered vaccinia virus maintains a level of expression of wild-type A13 and/or A27: for example, for improved viral assembly and/or adjusted viral influence on the complement cascade. In some alternative embodiments of the invention, therefore, the A13L or A27L fusion constructs are recombined into the J2R/TK gene (FIG. 6). In this way, (most of) the TK coding sequence is removed, which can be beneficial for the reasons already discussed and known to the skilled person. However, in this case, the wild-type copy of the respective genes (A13L and/or A27) will still be present and so at least some of the A13 or A27 protein expressed by the virus will be wild-type. On the other hand, the presence of the wild-type A13 and A27 genes provides a template for homologous recombination with the inserted A13L/A27L fusion constructs, which is generally undesirable. The plasmid constructs used to insert A13L and A27L fusion sequences into the TK locus particularly, may therefore contain A13L and A27L nucleotide sequences that are codon optimised for expression in human cells, thus diminishing the possibility of non-intended recombination with the wild-type gene sequence.

BSC40 cells were transfected with the linearised targeting constructs using Lipofectamine 2000 followed by infection (separately) with various vaccinia virus strains, including Copenhagen (COP), Western Reserve (WR) and Lister to achieve recombination with the viral genome of each of the vaccinia strains. Resulting cell lysates were titrated on BSC40 cell layers overlayed with 1.0% methylcellulose or bacto-agar/DMEM containing G418. mCherry-positive clones were plaque purified through 3 or 4 rounds of selection of fluorescent plaques in G418.

Correct insertion of the transgene gene fusions was confirmed by PCR and gene sequencing.

### Constructs

In order to prepare fusion constructs for homologous recombination, the following A13-gene fusions (Table 1) were synthesised by Genewiz and inserted into the pUC57-Amp plasmid:

TABLE 1
A13L fusion contructs. The A13L-x-CD55₍₃₅₋₂₈₄₎ and A13L-CD55₍₃₅₋₂₈₄₎-V5 gene
fusion constructs were prepared in order to establish (i) that the same/similar
technical effect can be achieved with or without a flexible peptide linker
between the fusion partners, and (ii) that irrespective of the presence of
C-terminal identification tag. The nucleotide sequences of SEQ ID NO: 37 to
46 encode corresponding A13 fusion proteins according to SEQ ID NOs: 47 to
56, respectively. V5 peptide tag (SEQ ID NOs: 69 (nt), 70 (prt).

| Fusion construct | Name | SEQ ID NO: |
|---|---|---|
| VCP gene sequence attached to the 3′ end of the | A13L-VCP | 37 |
| A13L gene via a linker sequence encoding a GGGGS | | |
| peptide | | |
| Two VCP gene sequences attached to the 3′ end of | A13L-VCPx2 | 38 |
| the A13L gene via a linker sequence encoding a | | |
| GGGGS peptide | | |
| Four VCP gene sequences attached to the 3′ end of | A13L-VCPx4 | 39 |
| the A13L gene via a linker sequence encoding a | | |
| GGGGS peptide | | |
| A mutant VCP gene sequence attached to the 3′ end | A13L-VCPmut | 40 |
| of the A13L gene via a linker sequence encoding a | | |
| GGGGS peptide | | |
| A CD55 gene sequence attached to the 3′ end of the | A13L-CD55₍₃₅₋₂₈₄₎ | 41 |
| A13L gene via a linker sequence encoding a GGGGS | | |
| peptide | | |
| A CD35 gene sequence attached to the 3′ end of the | A13L-CD35₍₄₂₋₁₅₈₄₎ | 42 |
| A13L gene via a linker sequence encoding a GGGGS | | |
| peptide | | |
| A CCPH gene sequence attached to the 3′ end of the | A13L-CCPH₍₂₁₋₂₆₆₎ | 43 |
| A13L gene via a linker sequence encoding a GGGGS | | |
| peptide | | |
| An ORF4 gene sequence attached to the 3′ end of | A13L-ORF4₍₂₂₋₂₆₈₎ | 44 |
| the A13L gene via a linker sequence encoding a | | |
| GGGGS peptide | | |
| A CD55 gene sequence directly attached to the 3′ | A13L-x-CD55₍₃₅₋₂₈₄₎ | 45 |
| end of the A13L gene without a linker sequence | | |
| A CD55 gene sequence attached to the 3′ end of the | A13L-CD55₍₃₅₋₂₈₄₎-V5 | 46 |
| A13L gene via a linker sequence encoding a GGGGS | | |
| peptide with a 3′ sequence encoding a V5 peptide tag | | |

Similarly, in order to prepare further fusion constructs for homologous recombination, the following A27-gene fusions (Table 2) were synthesised by Genewiz and inserted into the pUC57-Amp plasmid:

TABLE 2
A27L fusion contructs. The nucleotide sequences of SEQ
ID NO: 57 to 62 encode corresponding A13 fusion proteins
according to SEQ ID NOs: 63 to 68, respectively.

| Fusion construct | Name | SEQ ID NO: |
|---|---|---|
| VCP gene sequence attached to the 5′ end of the | VCP-A27L | 57 |
| A13L gene via a linker sequence encoding a | | |
| (GGGGS)₃ peptide | | |
| A mutant VCP gene sequence attached to the 5′ end | VCPmut-A27L | 58 |
| of the A13L gene via a linker sequence encoding a | | |
| (GGGGS)₃ peptide | | |
| A CD55 gene sequence attached to the 3′ end of the | CD55₍₃₅₋₂₈₄₎-A27L | 59 |
| A13L gene via a linker sequence encoding a | | |
| (GGGGS)₃ peptide | | |
| A CD35 gene sequence attached to the 3′ end of the | CD35₍₄₂₋₁₅₈₄₎-A27L | 60 |
| A13L gene via a linker sequence encoding a | | |
| (GGGGS)₃ peptide | | |
| A CCPH gene sequence attached to the 3′ end of the | CCPH₍₂₁₋₂₆₆₎-A27L | 61 |
| A13L gene via a linker sequence encoding a | | |
| (GGGGS)₃ peptide | | |
| An ORF4 gene sequence attached to the 3′ end of | ORF4₍₂₂₋₂₆₈₎-A27L | 62 |
| the A13L gene via a linker sequence encoding a | | |
| (GGGGS)₃ peptide | | |

A13 and A27 protein sequences from the vaccinia Copenhagen strain were used in this Example. However, the A13L and A27L gene sequences from any other desired vaccinia strain could be used to engineer other strains of vaccinia virus, particularly since all gene sequences are orthologous and are expected to behave similarly. By way of demonstration, sequence alignments of A13 and A27 from different vaccinia strains are shown in FIGS. 3 and 4, respectively, and demonstrate how well conserved the sequences of these genes are across viral genomes. In addition, the A13 and A27 protein sequences may be modified/mutated in some embodiments if it is desired to modify the natural behaviour of the protein in some way, or, for example, to achieve an efficient or optimal fusion with its fusion partner.

In various other embodiments, fusion constructs without GGGGS linkers between the respective domains are encompassed, as well as fusion constructs with different flexible or structured linkers to those described herein; provided that each member of the fusion pair is able to perform its intended function.

To create gene targeting constructs the respective inserts were excised and cloned into a suitable targeting vector, particularly a bacterial plasmid, for example pUC57, which contains a positive mCherry-Neo selection cassette and appropriate homolgous recombination sequences for recombining into the desired locus of the vaccinia virus.

Gene targeting by homologous recombination, followed by virus selection and purification resulted in the following 29 engineered vaccinia viruses (Table 3) where Env protein 1 is A13 and Env Protein 2 is A27. In each of the below embodiments, the A13L-fusion construct was inserted into the corresponding A13L gene locus, and the A27L-fusion construct was inserted into the corresponding A27L gene locus to create the engineered viruses.

TABLE 3
Engineered vaccinia virus strains according to the disclosure. Each
engineered vaccinia virus vector contains an A13L-fusion construct
to express an A13-fusion protein (engineered strain (STV) numbers
1 to 5, 10 to 18, 21 to 25 and 28); an A27L-fusion construct to express
an A27-fusion protein (engineered strain numbers 6, 8 and 29): or
both an A13L-fusion construct and an A27L-fusion construct to express
an A13-fusion protein and an A27-fusion protein in the same virion
(engineered strain numbers 7, 9, 19, 20, 26 and 27).

| | Env Protein 1 | Env Protein 2 |
|---|---|---|
| Strain No. | (A13-fusion) | (A27-fusion) |
| STV0001 | GFP | |
| STV0002 | VCP | |
| STV0003 | VCPmut | |
| STV0004 | CD55 | |
| STV0005 | CD35 | |
| STV0006 | | VCP |
| STV0007 | CD55 | VCP |
| STV0008 | | CD55 |
| STV0009 | VCP | CD55 |
| STV0010 | VCPx2 | |
| STV0011 | VCPx4 | |
| STV0012 | VCP-CD55 | |
| STV0013 | CD55-VCP | |
| STV0014 | CCPH | |
| STV015 | CCPHx2 | |
| STV016 | CCPHx4 | |
| STV017 | VCP-CCPH | |
| STV018 | CCPH-VCP | |
| STV019 | CCPH | VCP |
| STV020 | VCP | CCPH |
| STV021 | ORF4 | |
| STV022 | ORF4x2 | |
| STV023 | ORF4x4 | |
| STV024 | VCP-ORF4 | |
| STV025 | ORF4-VCP | |
| STV026 | ORF4 | VCP |
| STV027 | VCP | ORF4 |

### Example 2

### Virus Purification and Quantitation

HeLa cells were grown in T175 flasks containing complete DMEM containing 5% FBS. When 50-70% confluency was achieved cells were washed once with PBS and infected with 5 ml serum-free media containing virus (MOI=0.02). After 1.5 hours the inoculum was removed and replaced with 20 ml complete media. At 48 to 72 hours post-infection cells were scraped in media and spun at 1,500 rpm for 5 minutes at 4° C. The cell pellet was washed twice in cold PBS before resuspension in 10 ml of 10 mM Tris-HCl pH9 and incubated on ice for 15 minutes. The suspension was lysed on ice using a Dounce homogeniser.

Cell nuclei were pelleted for 5 minutes at 1,500 rpm at 4° C., and the supernatant (called the post-nuclear supernatant, PNS) loaded in ultracentrifuge tubes on an equal volume of 36% sucrose (w/v) in 10 mM Tris-HCl pH9.

Virus was pelleted for 90 minutes at 24,000 rpm, and the supernatants discarded and resuspended in 1 mM Tris-HCl pH9 and aliquots frozen at −80° C. Aliquots were titrated on BSC40 cells. BSC40 cells were grown to confluency in 6-well plastic plates before viral aliquots were diluted in a ten-fold series in 1 ml serum free media. The 6-well plastic plate samples were then washed with serum-free media and the inoculum was added. After 1.5 hours the media was removed, the wells washed twice with PBS, and overlayed with 1% methylcellulose in DMEM containing 5% FBS. After 3 days the overlay was removed by aspiration and the wells washed twice with PBS before fixation with 4% formalin. Wells were stained with 1% crystal violet in 20% ethanol for 30 minutes at room temperature, washed in water and dried.

Plaques were counted and the titre was calculated as plaque-forming units per ml.

### Example 3

### Protein Expression by Western Blot

To determine expression of full-strength envelope protein-complement control protein fusions, Western blot was performed.

Cells were washed once with ice-cold PBS and lysed in wells with 1× lithium dodecyl sulfate buffer supplemented with 0.5 M DTT and 1× proteinase/phosphatase inhibitor mixtures (Thermo Fisher Scientific). Cell lysates were sonicated for 1 min to reduce viscosity. Proteins were resolved on 4 to 12% NuPAGE Bis-Tris gels (Thermo Fisher Scientific) and transferred to nitrocellulose membranes by blotting. Membranes were blocked with 5% non-fat milk in Tris-buffered saline (TBS) for 1 hour and then incubated with anti-V5 rabbit antibody (Clone Poly29038 Biolegend) diluted in 5% non-fat milk in TBS with 0.1% Tween 20 (TBST) overnight at 4° C. The membranes were washed three times with TBS and then treated with Goat Anti-Rabbit IgG H&L (HRP) (Abcam ab6721) diluted 1/3000 in TBST containing 5% non-fat milk for 1 hour at room temperature. The membrane was washed three times with TBS with 0.1% Tween 20 (TBST) and twice with water.

Bound proteins were detected using Novex™ HRP Chromogenic Substrate (TMB) (Invitrogen™ WP20004).

### Example 4

### Virus Growth Analysis

### Plaque Morphology

In some cases, envelope protein fusions may alter viral replication, infectivity and spread which may be apparent, e.g. from altered plaque morphology, which can be determined using the following method.

Viruses were titrated by serial dilution on BSC40 cells and overlayed with 1% methylcellulose semi-solid media as above. The appropriate dilution was established that results in 20 to 50 plaques per well of a 6-well tissue culture plate, and these were used to determine the average plaque size.

Representative images of plaques as formed by the respective virus strains in BSC40 cells were visualised with crystal violet 4 days post infection. The diameters of at least 125 individual plaques from such low-density titrations were determined at about 48 hours postinfection (hpi), using NIH ImageJ software. Quantitative analysis of infectious virus (plaque forming units per ml) present in the culture supernatant of the indicated virus strains 24 hours post-infection is then determined using standard plaque assays on BSC40 cells.

The results (not shown) indicate that none of the viruses containing modified A13L genes exhibit a multiplication defect that results in a reduced size plaque compared to the unmodified gene and the A13L-EGFP fusion virus.

### Step Growth Analysis

The replicative ability of candidate viruses was also determined by performing growth curves.

For the one-step curve HeLa cells were infected in 6-well plates at a multiplicity of infection of 10 (MOI=10), whereas for the multi-step curve cells were infected with a MOI of 0.02. Cells from individual wells were harvested at 0, 2, 4, 6, 8, 12, 16 and 24 hours post-infection (one-step) or at 0, 8, 12, 24, 48, 72 and 96 hours post-infection (multi-step) in 1 ml of serum free media. For multi-step growth the released virus population was collected with the overlaying media before the cells were harvested in 1 ml of media. Supernatants and scraped cells were each freeze-thawed separately three times, spun down at 13,000 k for 30 minutes and the resulting clarified supernatants were titrated on BSC40 cells in triplicate. The resulting infectivity was plotted (pfu/ml) and the data revealed that all A13 fusion protein expressing viruses exhibited both intracellular multiplication and cell-to-cell spread at levels comparable to parental viruses (i.e. comparable viruses lacking the relevant A13L fusion gene construct).

### Example 5

### In Vitro Complement Inhibition Assays

### Human Serum Assay

The ability of protein fusion variants of the vaccinia mature virion (MV) to escape serum complement inhibition was investigated in human serum.

Viruses (2×10⁴ to 2×10⁷ pfu) were incubated in serum-free media containing 50% pooled human complement sera (Merck S1764) for 60 minutes at 37° C. Control serum samples were heat-inactivated for 30 minutes at 56° C. To determine the level of infectivity samples were serially diluted in 1 ml serum-free DMEM and added to confluent BSC40 cells in 6-well plastic plates for 1 hour. After this the media was removed, the wells washed twice with PBS, and overlayed with 1% methylcellulose in DMEM containing 5% FBS. After 2 days the overlay was removed by aspiration, the wells washed twice with PBS before fixation with 4% formaldehyde (Merck). Wells were stained with 1% crystal violet in 15% ethanol for 30 minutes at room temperature, washed in water and dried.

Plaques were counted and the percentage of neutralisation calculated.

Pooled human serum inhibited infectivity of wild-type Copenhagen viral strain and the A13L-EGFP mutant virus strain by over 80%. However, when virus was incubated with heat-inactivated serum less than 10% inhibition of viral infectivity was observed.

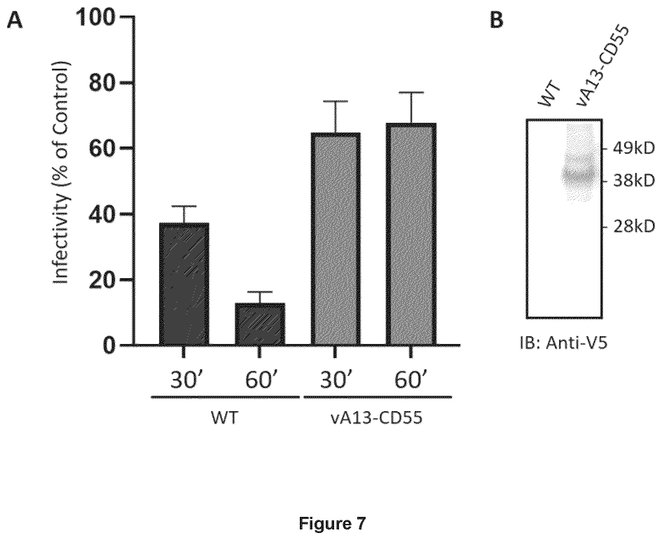

Corresponding experiments were performed with each of the following engineered viruses described herein expressing the following fusions: A13-VCP, A13-VCPmut, A13-CD55, A13-CD35, A13-Compstatin, A13-CCPH and A13-ORF4. The results (not shown) indicate that each of the test viruses expressing complement-inhibiting fusion proteins of this disclosure were significantly able to retain infectivity in the presence of complement containing pooled human serum. FIG. 7 is provided by way of example, and illustrates the results of incubating either vA13-CD55/A13-CD55 or a corresponding wild-type (WT) virus with human serum, and demonstrates that an engineered virus of the present disclosure exhibits a significant increase in resistance to serum-mediated neutralisation. Furthermore, improved retention of infectivity relative to wild-type and reporter viruses was also observed in VCP-A27, SPICE-A27, CD55-A27 and CD35-A27, Compstatin-A27, CCPH-A27 amd ORF4-A27 viruses, demonstrating that the engineered vaccinia viruses of the disclosure exhibit improved complement avoidance in human serum. Moreover, the results indicate that either the A13 or A27 envelope proteins are suitable candidates for fusion proteins designed to avoid complement-induced inactivation.

### Rabbit Complement Assay

The ability of MV protein fusion variants to escape serum complement inhibition was also investigated in rabbit serum.

Viruses (2×10⁴ to 2×10⁷ pfu) were incubated in 1 ml PBS containing 50% rabbit complement (Merck S7764) for 60 minutes at 37° C. Control serum samples were heat-inactivated for 30 minutes at 56° C. To determine the level of infectivity samples were serially diluted in 1 ml serum-free DMEM and added to confluent BSC40 cells in 6-well plastic plates for 1 hour. After this the media was removed, the wells washed twice with PBS, and overlayed with 1% methylcellulose in DMEM containing 5% FBS. After 2 days the overlay was removed by aspiration, and the wells washed twice with PBS before fixation with 4% formaldehyde. Wells were stained with 1% crystal violet in 15% ethanol for 30 minutes at room temperature, washed in water and dried.

Plaques were counted and the percentage of neutralisation calculated.

As in the above-described assays using human serum, rabbit serum inhibited infectivity of wild-type and A27-GFP and A13-GFP viruses by over 80%, whereas virus incubation with heat-inactivated serum resulted in less than 10% inhibition. The results indicate that the test, mutant viruses: A13-VCP, A13-VCPmut, A13-CD55, A13-CD35, A13-Compstatin, A13-CCPH and A13-ORF4 were all significantly able to retain infectivity in the presence of complement containing rabbit serum. This effect was also observed in VCP-A27, SPICE-A27, CD55-A27 and CD35-A27, Compstatin-A27, CCPH-A27 amd ORF4-A27 viruses, again demonstrating that the engineered vaccinia viruses of the disclosure exhibit improved complement avoidance in rabbit serum.

### Antibody Neutralisation

The ability of MV-fusion mutants to escape antibody neutralisation was investigated.

BSC40 cells were seeded in 6-well plates and infected when they reached confluence. Viruses (2×10⁴ pfu) were diluted in 1 ml serum-free media in the presence or absence of 50% rabbit complement sera and incubated for 1 hour at 37° C. with 40 μg of the following antibodies: 9503-2057 (BioRad), PA1-7258 (Invitrogen), both anti-Vaccinia; and the control Rabbit IgG antibody ab37415 (Abcam). Serial dilutions were prepared in 1 ml serum-free media and added to BSC40 containing wells. After 2 hours the inoculum was removed, the wells were washed twice with PBS, and overlayed with 1% methylcellulose in complete DMEM containing 5% FBS. After 2 days the overlay was removed by aspiration, the wells washed twice with PBS before fixation with 4% formaldehyde. Wells were stained with 1% crystal violet in 15% ethanol for 30 minutes at room temperature, washed in water and dried.

Plaques were counted and the percentage of neutralisation calculated.

The results indicate the fusion constructs reduce antibody mediated neutralisation in the presence of complement.

Many modifications may be made to the above examples without departing from the scope of the present invention as defined in the accompanying claims.

### DISCUSSION AND CONCLUSIONS

Vaccinia virus fusion proteins/chimeric genes have been designed that can be expressed in the virus and localise in the viral envelope. The fusion proteins comprise one portion (or domain) that is a natural (or engineered) viral protein sequence, and one portion (or domain) that is a heterologous gene.

As disclosed herein, the viral gene portion of the fusion/chimeric gene may encode the vaccinia viral protein A13 or A27, which are expressed and found with different distribution patterns, in the viral envelope. While A13 is widely distributed around the viral envelope, A27 exhibits a more polarised distribution.

In these Examples, the heterologous gene is a complement regulator (or complement regulatory) protein, such as VCP, SPICE etc. as described elsewhere herein, which are known to inhibit complement activity. As such, it was hypothesised that A13- and/or A27-fusion proteins may enable an engineered vaccinia virus particle to evade complement-mediated inactivation, provided that such fusion proteins are suitable expressed and displayed on the viral envelope in order that their VCP (or functionally equivalent domain) may interact with complement.

Fusion/chimeric nucleic acid construct sequences encoding fusion proteins comprising the relevant vaccinia envelope protein, A13 and/or A27 with one or various complement-inhibitory peptide domains have been produced and used to express engineered vaccinia virus particles. Studies of the resultant engineered viruses demonstrate that complement-inhibitory fusion proteins-encoding nucleic acids can be inserted into various vaccinia viral vectors, and the encoded fusion proteins caused to be expressed and displayed on the envelope of the engineered vaccinia virus.

As the person skilled in the art understands, by appropriate design of the fusion/chimeric nucleic acid sequences it is possible to insert the heterologous chimeric gene construct in any desired location within a viral vector. In these studies, chimeric gene constructs have been inserted into the thymidine kinase (TK) gene locus (J2R), thus simultaneously creating a vaccinia virus TK deletion mutant. Vaccinia virus TK deletion mutants may exhibit beneficial behaviours for disease therapy—particularly in regard to the treatment of proliferative disorders (such as cancers)—as they must rely on host cell TK activity for replication, which activity is significantly more prevalent in proliferating cells compared to quiescent cells. By inserting chimeric genes of the disclosure into the TK gene locus, the viral vector (unless otherwise further modified) will retain its wild type A13L and/or A27L gene and associated expression elements, such that engineered viruses in these aspects and embodiments may express a mixture of wild type envelope protein (i.e. A13 or A27) as well as a corresponding heterologous fusion protein comprising an A13 or A27 protein sequence fused to a complement regulatory protein sequence. In this way, beneficial traits of wild type virus replication, assembly and infectivity may, in some embodiments, be better maintained than comparative engineered viruses wherein all of the expressed A13 or A27 protein sequences are fused to a complement regulatory protein.

In some aspects and embodiments, the chimeric gene constructs are designed to insert the heterologous gene sequence into the corresponding envelope protein gene locus, such that A13-fusion protein encoding genes are inserted into and replace the A13L wild type gene, and A27-fusion protein encoding genes are inserted into and replace the A27L wild type gene. Whilst in such embodiments all of the respective wild type protein is removed from the resultant engineered virus particle, the present studies have demonstrated that such engineered virus particles—whether containing A13- or A27-fusion proteins are able to replicate, assemble and infect cells. Moreover, such engineered viruses may be beneficial, according to some aspects and embodiments, in displaying higher numbers/concentration of the complement regulatory protein on its surface and, thus, exhibiting stronger complement avoidance/resistance to inactivation when compared to engineered viruses that retain expression of a proportion of wild type A13 or A27, respectively, on its surface. As such, these two different approaches to the creation of engineered viral vectors offer alternative strategies for complement avoidance and the creation of optimal engineered viruses for any particular therapeutic use.

These studies further demonstrate that while the exact point of fusion of a complement regulatory protein sequence with an A13 or A27 protein sequence does not appear to critical to the efficacy of the resultant protein and corresponding engineered virus particle, the data shows that novel, viable complement regulatory fusion proteins can be produced by fusion of a complement regulatory protein sequence at (or close to) the C-terminus of an A13 protein; and novel, viable complement regulatory fusion proteins can also be produced by fusion of a complement regulatory protein sequence at (or close to) the N-terminus of an A27 protein.

Moreover, it has been established that various engineered virus particles can be produced to express a wide range of different fusion proteins on their surface (particularly on the surface of the MV particle). The fusion proteins may comprise any one of several different complement regulatory protein sequences (as disclosed herein) fused with a viral envelope protein sequence (particularly A13 and/or A27), and each such fusion protein can impart complement inhibitory/avoidance activity to the engineered virus particle.

Accordingly, the present disclose describes the production of a range of novel engineered viruses, as well as corresponding viral vectors, nucleic acid sequences and novel fusion proteins, which show promise as therapeutic agents. In particular, the therapeutic agents of this disclosure may have utility for the treatment of various proliferative disorders and cancers (as described above).

TABLE 4
Nucleic Acid and Peptide Sequences.
SEQUENCES

| SEQ | | |
|---|---|---|
| ID | | |
| NO: | Sequence Name | Sequence |
| 1 | Vaccinia virus A13 | MIGILLLIGICVAVTVAILYSMYNKIKNSQNPNPSPNLNSPPPEPKNTK |
| | (prt) | FVNNLEKDHISSLYNLVKSSV |
| 2 | CMLV130 | MIGILLLIGICVAVTVAILYAMYNKIKNSQNPNPSPNVNSPPSETRNTK |
| | (Camelpox virus | FVNNLEKDHISSLYNLVKSSA |
| | A13) (prt) | |
| 3 | VARVgp117 | MIGILLLIGICVAVTVAILYAMYNKIKNSQNPSPNVNLPPPETRNTRFV |
| | (Variola virus A13) | NNLEKDHISSLYNLVKSSV |
| | (prt) | |
| 4 | CPXV145 protein | MIGILLLIGICVAVTVAILAMYNKIKNSQNPNPSPNLNSPPPETRNTKF |
| | (Cowpox virus | VNNLEKDHISSLYNLVKSSA |
| | A13) (prt) | |
| 5 | Taterapox virus | MIGILLLIGICVAVTVAILYAMYNKIKNSQNPSPNVNSPPPETRNTKFV |
| | A13 (prt) | NNLEKDHISSLYNLVKSSA |
| 6 | A14L (Monkeypox | MIGILLLIGICVAVTVAILYTLYNKIKNPQNPNPSPNLNSPPPETRNTK |
| | virus Zaire-96-1-16 | FVNNLEKDHISSLYNLVKSSA |
| | A13) (prt) | |
| 7 | Volepox virus A13 | MIGTIILIVICVTITAAIIYALYNRTKNQQKONTNTPSPEPRNTKFVNN |
| | (prt) | LNKDHITSLYNLVKSSS |
| 8 | Akhmeta virus A13 | MIGILLLIGICVAVTVAILYALYNKIKNPQNPNPNPNPNLNSPPPETRN |
| | (prt) | TKEVNNLEKEHISSLYNLVKSSA |
| 9 | EVM116 | MIGILLLIGICVAVTVAILYALYNKIKSPQNPDPNPPPPEPRNTKFVNN |
| | (Ectromelia virus | LEKDHISSLYNLVKSSA |
| | A13) (prt) | |
| 10 | Orthopoxvirus | MIGILLLIGICVAVTVAILYALYNKIKNPQNPDPNPPPPEPRNTKFVNN |
| | Abatino virus A13 | LEKDHISSLYNLVKSSA |
| | (prt) | |
| 11 | Skunkpox virus | MIGTIILIVICVTITAAIIYALYNRTKSQQKPNPPAPTPEPRNTKFVNN |
| | A13 (prt) | LEKDHITSLYNLVKSSS |
| 12 | Raccoonpox virus | MIGTIILIIICVTITAAIIYALYNRTKNQQNQNPPAPSPEPRNTKFVNN |
| | A13 (prt) | LEKDHITSLYNLVKSSS |
| 13 | Yokapox virus A13 | MILSIILIGVCIVIVGGIIYAVYYRNKQRPNNSNNNKKTKYVNSLGKEH |
| | (prt) | IDSLYNLFKSSP |
| 14 | Murmansk | MIGNIILIVLCVVVVGGIIYALYHRTQKQPNPPPNNDRKTKYVNSLGKE |
| | poxvirus A13 (prt) | HISSLYNLFKSSS |
| 15 | NY_014 poxvirus | MIGNIILIVLCVVVVGGIIYALYHRTQKQPNPPPNNDMKTKYVNSLGKE |
| | A13 (prt) | HISSLYNLFKSSS |
| 16 | Yaba monkey | MIADIILVIICVAIVGLIVYGVYNKKSANLFNEKQEEYKIEDIKTSYVD |
| | tumor virus A13 | RLKPNHLNSFYKLESGQFN |
| | (prt) | |
| 17 | Vaccinia | MDGTLFPGDDDLAIPATEFFSTKADKKPEAKREAIVKADEDDNEETLKQ |
| | Copenhagen virus | RLTNLEKKITNVTTKFEQIEKCCKRNDEVLFRLENHAETLRAAMISLAK |
| | A27 (prt) | KIDVQTGRRPYE |
| 18 | Vaccinia virus A27 | MDGTLFPGDDDLAIPATEFFSTKAAKKPEAKREAIVKADEDDNEETLKQ |
| | (prt) | RLTNLEKKITNVTTKFEQIEKCCKRNDEVLFRLENHAETLRAAMISLAK |
| | | KIDVQTGRRPYE |
| 19 | CPXV162 | MDGTLFPGDDDLAIPATEFFSTKAAKKPEAKREAIVKAEGDDNEETLKQ |
| | (Cowpox virus | RLTNLEKKITNVTTKFEQIEKCCKRNDEVLFRLENHAETLRAAMISLAK |
| | A27) (prt) | KIDVQTGRRPYE |
| 20 | CMLV146 | MDGTLFPGDDDLAIPATEFFSTKAAKKPEAKREAIIKADGDDNEETLKQ |
| | (Camelpox virus | RLTNLEKKITNVTTKFEQIEKCCKRNDEVLFRLENHAETLRAAMISLAK |
| | A27) (prt) | KIDVQTGRRPYDNLTLLFN |
| 21 | VARVgp134 | MDGTLFPGDDDLAIPATEFFSTKAAKKPEAKREAIVKADGDNNEETLKQ |
| | (Variola virus A27) | RLTNLEKKITNVTTKFEQIEKCCKRNDDVLFRLENHAETLRAAMISLAK |
| | (prt) | KIDVQTGRRPYE |
| 22 | Orthopox Abatino | MDGTLFPGDDDLAIPATEFFSTKAAKKPEAKREAIVKADGDDNEETLKQ |
| | virus A27 (prt) | RLTNLEKKINNVTTKFEQIEKCCKRNDEVLFRLENHAETLRAAMISLAK |
| | | KIDVQTGRRTYE |
| 23 | Akhmeta virus A27 | MDGTLFPGDDDLAIPATEFESTKAAKKIEAKREVIVKADGDDSEETLKQ |
| | (prt) | RLTNLEKKITNVTTKFEQIEKCCKRNDEVLFRLENHAETLRAAMISLAK |
| | | KIDVQTGRRPYE |
| 24 | EVM129 | MDGTLFPGDDDLAIPATEFFSTKAAKKPEDKHEATVKADGDDNEETLKQ |
| | (Ectromelia virus | RLTNLEKKITNVTTKFEQIEKCCKRNDEVLFRLENHAETLRAAMISLAK |
| | A27) (prt) | KIDVQTGRRPYE |
| 25 | Taterapox virus | MDGTLFPGDDDLAIPATEFESTKAAKKPEAKREAIVKADGYDNEETLKQ |
| | A27 (prt) | RLTNLEKKITNVTIKFEQIEKCCKRNDEVLFRLENHAETLRAAMISLAK |
| | | KIDVQTGRHPYE |
| 26 | A29L Monkeypox | MDGTLFPGDDDLAIPATEFESTKAAKNPETKREAIVKAYGDDNEETLKQ |
| | virus A27 (prt) | RLTNLEKKITNITTKFEQIEKCCKHNDEVLFRLENHAETLRAAMISLAK |
| | | KIDVQTGRRPYE |
| 27 | Volepox virus A27 | MDGTLFPGDDDIAIPATEFFVNRSAKKPEESVKSKVSKQKRKAVVKADG |
| | (prt) | EDDPDEDDDDDEEEEDDETIKDRLTNLEKKITNVTTKFAQIEKCCKRND |
| | | EVLFRLENHAETLRAAMLALAKKIDVQTGRORYE |
| 28 | Raccoonpox virus | MEGTLFPGDDDIAIPATEFFVNKAAKKPEKPAKRKVVKADDAEEKADEE |
| | A27 (prt) | EDAEEDIKGRLTNLEKKITNVTTKFAQIEKCCKRNDEVLFRLENHAETL |
| | | RVAMLSLAKKIDIQTGRQRYE |
| 29 | Skunkpox virus | MDGTLFPGDDDMAIPATEFFVTRAAKKPEEPVKRKVVKNKNKHKVVKAD |
| | A27 (prt) | GEDDPDEDDEDDDDEEDDDAEETIKHRLTNLEKKITNVTTKFAQIEKCC |
| | | KRNDEVLFRLENHAETLRAAMLTLAKKIDVQTGRQRYE |
| 30 | VCP (prt) | SCTIPSRPINMKFKNSVETDANANYNIGDTIEYLCLPGYRKQKMGPIYA |
| | | KCTGTGWTLFNQCIKRRCPSPRDIDNGOLDIGGVDFGSSITYSCNSGYH |
| | | LIGESKSYCELGSTGSMVWNPEAPICESVKCQSPPSISNGRHNGYEDFY |
| | | TDGSVVTYSCNSGYSLIGNSGVLCSGGEWSDPPTCQIVKCPHPTISNGY |
| | | LSSGFKRSYSYNDNVDFKCKYGYKLSGSSSSTCSPGNTWKPELPKCVR |
| 31 | Modified VCP (prt) | SCTIPSRPINMKFKNSVETDANANYNIGDTIEYLCLPGYRKQKMGPIYA |
| | | KCTGTGWTLFNQCIKRRCPSPRDIDNGHLDIGGVDFGSSITYSCNSGYH |
| | | LIGESKSYCELGSTGSMVWNPKAPICESVKCQSPPSISNGRHNGYEDFY |
| | | TDGSVVTYSCNSGYSLIGNSGVLCSGGEWSDPPTCQIVKCPHPTISNGY |
| | | LSSGFKRSYSYNDNVDFKCKYGYKLSGSSSSTCSPGNTWKPELPKCVR |
| 32 | SPICE (Variola | LSCCTIPSRPINMTFKNSVETDANANYNIGDTIEYLCLPGYRKQKMGPI |
| | virus VCP) (prt) | YAKCTGTGWTLFNQCIKRRCPSPRDIDNGHLDIGGVDFGSSITYSCNSG |
| | | YYLIGEYKSYCKLGSTGSMVWNPKAPICESVKCQLPPSISNGRHNGYND |
| | | FYTDGSVVTYSCNSGYSLIGNSGVLCSGGEWSNPPTCQIVKCPHPTILN |
| | | GYLSSGFKRSYSYNDNVDFTCKYGYKLSGSSSSTCSPGNTWQPELPKCV |
| | | R |
| 33 | MOPICE | LSYCTIPSRPINMKFKNSVETDANYNIGDTIEYLCLPGYRKQKMGPIYA |
| | (Monkeypox virus | KCTGTGWTLFNQCIKRRCPSPRDIDNGOLDIGGVDFGSSITYSCNSGYH |
| | Zaire-96-1-16 | LIGESKSYCELGSTGSMVWNPEAPICESVKCQSPPSISNGRHNGYEDFY |
| | VCP) (prt) | IDGSIVTYSCNSGYSLIGNSGVMCSGGEWSNPPTCQIVKCPHPISNGKL |
| | | LAA |
| 34 | Linker (prt) | GGGGS |
| 35 | Linker (prt) | GGGGSGGGGS |
| 36 | Linker (prt) | GGGGSGGGGSGGGGS |
| 37 | A13L-VCP (nt) | atgattggtattcttttgttgatcggtatttgcgtagcagttaccgtcg |
| | | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaggaggaggtggatcctcctgtactattccgtcacg |
| | | acccattaatatgaaatttaagaatagtgtggagactgatgctaatgct |
| | | aattacaacataggagacactatagaatatctatgtctacctggataca |
| | | gaaagcaaaaaatgggacctatatatgctaaatgtacaggtactggatg |
| | | gacactctttaatcaatgtattaaacggagatgcccatcgccacgagat |
| | | atcgataatggccaacttgatattggtggagtagactttggctctagta |
| | | taacgtactcttgtaatagcggatatcatttgatcggtgaatctaaatc |
| | | gtattgtgaattaggatctactggatctatggtatggaatcccgaggca |
| | | cctatttgtgaatctgttaaatgccaatcccctccatctatatccaacg |
| | | gaagacataacggatacgaggatttctacaccgatgggagcgttgtaac |
| | | ttatagttgcaatagtggatattcgttgattggtaactctggtgtcctg |
| | | tgttcaggaggagaatggtccgatccacccacgtgtcagattgttaaat |
| | | gtccacatcctacaatatcaaacggatacttgtctagcgggtttaaaag |
| | | atcatactcatacaacgacaatgtagactttaagtgcaagtacggatat |
| | | aaactatctggttcctcatcatctacttgctctccaggaaatacatgga |
| | | agccggaacttccaaaatgtgtacgc |
| 38 | A13L-VCPx2 (nt) | atgattggtattcttttgttgatcggtatttgcgtagcagttaccgtcg |
| | | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaGGCGGAGGTGGAAGTtcctgtactattccgtcacg |
| | | acccattaatatgaaatttaagaatagtgtggagactgatgctaatgct |
| | | aattacaacataggagacactatagaatatctatgtctacctggataca |
| | | gaaagcaaaaaatgggacctatatatgctaaatgtacaggtactggatg |
| | | gacactctttaatcaatgtattaaacggagatgcccatcgcctcgagat |
| | | atcgataatggccaacttgatattggtggagtagactttggctctagta |
| | | taacgtactcttgtaatagcggatatcatttgatcggtgaatctaaatc |
| | | gtattgtgaattaggatctactggatctatggtatggaatcccgaggca |
| | | cctatttgtgaatctgttaaatgccaatcccctccatctatatccaacg |
| | | gaagacataacggatacgaggatttCtaCaccgatgggagcgttgtaac |
| | | ttatagttgcaatagtggatattcgttgattggtaactctggtgtcctg |
| | | tgttcaggaggagaatggtccgatccacccacgtgtcagattgttaaat |
| | | gtccacatcctacaatatcaaacggatacttgtctagcgggtttaaaag |
| | | atcatactcatacaacgacaatgtagactttaagtgcaagtacggatat |
| | | aaactatctggttcctcatcatctacttgctctccaggaaatacatgga |
| | | agccggaacttccaaaatgtgtacgcGGCGGAGGTGGAAGTtcctgtac |
| | | tattccgtcacgacccattaatatgaaatttaagaatagtgtggagact |
| | | gatgctaatgctaattacaacataggagacactatagaatatctatgtc |
| | | tacctggatacagaaagcaaaaaatgggacctatatatgctaaatgtac |
| | | aggtactggatggacactctttaatcaatgtattaaacggagatgccca |
| | | tcgcctcgagatatcgataatggccaacttgatattggtggagtagact |
| | | ttggctctagtataacgtactcttgtaatagcggatatcatttgatcgg |
| | | tgaatctaaatcgtattgtgaattaggatctactggatctatggtatgg |
| | | aatcccgaggcacctatttgtgaatctgttaaatgccaatcccctccat |
| | | ctatatccaacggaagacataacggatacgaggatttCtaCaccgatgg |
| | | gagcgttgtaacttatagttgcaatagtggatattcgttgattggtaac |
| | | tctggtgtcctgtgttcaggaggagaatggtccgatccacccacgtgtc |
| | | agattgttaaatgtccacatcctacaatatcaaacggatacttgtctag |
| | | cgggtttaaaagatcatactcatacaacgacaatgtagactttaagtgc |
| | | aagtacggatataaactatctggttcctcatcatctacttgctctccag |
| | | gaaatacatggaagccggaacttccaaaatgtgtacgc |
| 39 | A13L-VCPx4 (nt) | atgattggtattcttttgttgatcggtatttgcgtagcagttaccgtcg |
| | | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaGGCGGAGGTGGAAGTtcctgtactattccgtcacg |
| | | acccattaatatgaaatttaagaatagtgtggagactgatgctaatgct |
| | | aattacaacataggagacactatagaatatctatgtctacctggataca |
| | | gaaagcaaaaaatgggacctatatatgctaaatgtacaggtactggatg |
| | | gacactctttaatcaatgtattaaacggagatgcccatcgcctcgagat |
| | | atcgataatggccaacttgatattggtggagtagactttggctctagta |
| | | taacgtactcttgtaatagcggatatcatttgatcggtgaatctaaatc |
| | | gtattgtgaattaggatctactggatctatggtatggaatcccgaggca |
| | | cctatttgtgaatctgttaaatgccaatcccctccatctatatccaacg |
| | | gaagacataacggatacgaggatttCtaCaccgatgggagcgttgtaac |
| | | ttatagttgcaatagtggatattcgttgattggtaactctggtgtcctg |
| | | tgttcaggaggagaatggtccgatccacccacgtgtcagattgttaaat |
| | | gtccacatcctacaatatcaaacggatacttgtctagcgggtttaaaag |
| | | atcatactcatacaacgacaatgtagactttaagtgcaagtacggatat |
| | | aaactatctggttcctcatcatctacttgctctccaggaaatacatgga |
| | | agccggaacttccaaaatgtgtacgcGGCGGAGGTGGAAGTtcctgtac |
| | | tattccgtcacgacccattaatatgaaatttaagaatagtgtggagact |
| | | gatgctaatgctaattacaacataggagacactatagaatatctatgtc |
| | | tacctggatacagaaagcaaaaaatgggacctatatatgctaaatgtac |
| | | aggtactggatggacactctttaatcaatgtattaaacggagatgccca |
| | | tcgcctcgagatatcgataatggccaacttgatattggtggagtagact |
| | | ttggctctagtataacgtactcttgtaatagcggatatcatttgatcgg |
| | | tgaatctaaatcgtattgtgaattaggatctactggatctatggtatgg |
| | | aatcccgaggcacctatttgtgaatctgttaaatgccaatcccctccat |
| | | ctatatccaacggaagacataacggatacgaggatttCtaCaccgatgg |
| | | gagcgttgtaacttatagttgcaatagtggatattcgttgattggtaac |
| | | tctggtgtcctgtgttcaggaggagaatggtccgatccacccacgtgtc |
| | | agattgttaaatgtccacatcctacaatatcaaacggatacttgtctag |
| | | cgggtttaaaagatcatactcatacaacgacaatgtagactttaagtgc |
| | | aagtacggatataaactatctggttcctcatcatctacttgctctccag |
| | | gaaatacatggaagccggaacttccaaaatgtgtacgcggcggaggtgg |
| | | aagttcctgtactattccgtcacgacccattaatatgaaatttaagaat |
| | | agtgtggagactgatgctaatgctaattacaacataggagacactatag |
| | | aatatctatgtctacctggatacagaaagcaaaaaatgggacctatata |
| | | tgctaaatgtacaggtactggatggacactctttaatcaatgtattaaa |
| | | cggagatgcccatcgcctcgagatatcgataatggccaacttgatattg |
| | | gtggagtagactttggctctagtataacgtactcttgtaatagcggata |
| | | tcatttgatcggtgaatctaaatcgtattgtgaattaggatctactgga |
| | | tctatggtatggaatcccgaggcacctatttgtgaatctgttaaatgcc |
| | | aatcccctccatctatatccaacggaagacataacggatacgaggattt |
| | | ctacaccgatgggagcgttgtaacttatagttgcaatagtggatattcg |
| | | ttgattggtaactctggtgtcctgtgttcaggaggagaatggtccgatc |
| | | cacccacgtgtcagattgttaaatgtccacatcctacaatatcaaacgg |
| | | atacttgtctagcgggtttaaaagatcatactcatacaacgacaatgta |
| | | gactttaagtgcaagtacggatataaactatctggttcctcatcatcta |
| | | cttgctctccaggaaatacatggaagccggaacttccaaaatgtgtacg |
| | | cggcggaggtggaagttcctgtactattccgtcacgacccattaatatg |
| | | aaatttaagaatagtgtggagactgatgctaatgctaattacaacatag |
| | | gagacactatagaatatctatgtctacctggatacagaaagcaaaaaat |
| | | gggacctatatatgctaaatgtacaggtactggatggacactctttaat |
| | | caatgtattaaacggagatgcccatcgcctcgagatatcgataatggcc |
| | | aacttgatattggtggagtagactttggctctagtataacgtactcttg |
| | | taatagcggatatcatttgatcggtgaatctaaatcgtattgtgaatta |
| | | ggatctactggatctatggtatggaatcccgaggcacctatttgtgaat |
| | | ctgttaaatgccaatcccctccatctatatccaacggaagacataacgg |
| | | atacgaggatttctacaccgatgggagcgttgtaacttatagttgcaat |
| | | agtggatattcgttgattggtaactctggtgtcctgtgttcaggaggag |
| | | aatggtccgatccacccacgtgtcagattgttaaatgtccacatcctac |
| | | aatatcaaacggatacttgtctagcgggtttaaaagatcatactcatac |
| | | aacgacaatgtagactttaagtgcaagtacggatataaactatctggtt |
| | | cctcatcatctacttgctctccaggaaatacatggaagccggaacttcc |
| | | aaaatgtgtacgc |
| 40 | A13L-VCPmut(nt) | atgattggtattcttttgttgatcggtatttgcgtagcagttaccgtcg |
| | | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaggaggaggtggatcctcctgtactattccgtcacg |
| | | acccattaatatgaaatttaagaatagtgtggagactgatgctaatgct |
| | | aattacaacataggagacactatagaatatctatgtctacctggataca |
| | | gaaagcaaaaaatgggacctatatatgctaaatgtacaggtactggatg |
| | | gacactctttaatcaatgtattaaacggagatgcccatcgccacgagat |
| | | atcgataatggccaccttgatattggtggagtagactttggctctagta |
| | | taacgtactcttgtaatagcggatatcatttgatcggtgaatctaaatc |
| | | gtattgtgaattaggatctactggatctatggtatggaatcccaaggca |
| | | cctatttgtgaatctgttaaatgccaatcccctccatctatatccaacg |
| | | gaagacataacggatacgaggatttctacaccgatgggagcgttgtaac |
| | | ttatagttgcaatagtggatattcgttgattggtaactctggtgtcctg |
| | | tgttcaggaggagaatggtccgatccacccacgtgtcagattgttaaat |
| | | gtccacatcctacaatatcaaacggatacttgtctagcgggtttaaaag |
| | | atcatactcatacaacgacaatgtagactttaagtgcaagtacggatat |
| | | aaactatctggttcctcatcatctacttgctctccaggaaatacatgga |
| | | agccggaacttccaaaatgtgtacgc |
| 41 | A13L-CD55₍₃₅₋₂₈₄₎ | atgattggtattcttttgttgatcggtatttgcgtagcagttaccgtcg |
| | (nt) | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaggaggaggtggatccgactgtggccttcccccaga |
| | | tgtacctaatgcccagccagctttggaaggccgtacaagttttcccgag |
| | | gatactgtaataacgtacaaatgtgaagaaagctttgtgaaaattcctg |
| | | gcgagaaggactcagtgatctgccttaagggcagtcaatggtcagatat |
| | | tgaagagttctgcaatcgtagctgcgaggtgccaacaaggctaaattct |
| | | gcatccctcaaacagccttatatcactcagaattattttccagtcggta |
| | | ctgttgtggaatatgagtgccgtccaggttacagaagagaaccttctct |
| | | atcaccaaaactaacttgccttcagaatttaaaatggtccacagcagtc |
| | | gaattttgtaaaaagaaatcatgccctaatccgggagaaatacgaaatg |
| | | gtcagattgatgtaccaggtggcatattatttggtgcaaccatctcctt |
| | | ctcatgtaacacagggtacaaattatttggctcgacttctagtttttgt |
| | | cttatttcaggcagctctgtccagtggagtgacccgttgccagagtgca |
| | | gagaaatttattgtccagcaccaccacaaattgacaatggaataattca |
| | | aggggaacgtgaccattatggatatagacagtctgtaacgtatgcatgt |
| | | aataaaggattcaccatgattggagagcactctatttattgtactgtga |
| | | ataatgatgaaggagagtggagtggcccaccacctgaatgcaga |
| 42 | A13L-CD35₍₄₂₋₁₅₈₄₎ | atgattggtattcttttgttgatcggtatttgcgtagcagttaccgtcg |
| | (nt) | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaGGCGGAGGTGGAAGTGGAGGCGGTGGATCCGGTGG |
| | | AGGAGGCAGCcaatgcaatgccccagaatggcttccatttgccaggcct |
| | | accaacctaactgatgaatttgagtttcccattgggacatatctgaact |
| | | atgaatgccgccctggttattccggaagaccgttttctatcatctgcct |
| | | aaaaaactcagtctggactggtgctaaggacaggtgcagacgtaaatca |
| | | tgtcgtaatcctccagatcctgtgaatggcatggtgcatgtgatcaaag |
| | | gcatccagttcggatcccaaattaaatattcttgtactaaaggataccg |
| | | actcattggttcctcgtctgccacatgcatcatctcaggtgatactgtc |
| | | atttgggataatgaaacacctatttgtgacagaattccttgtgggctac |
| | | cccccaccatcaccaatggagatttcattagcaccaacagagagaattt |
| | | tcactatggatcagtggtgacctaccgctgcaatcctggaagcggaggg |
| | | agaaaggtgtttgagcttgtgggtgagccctccatatactgcaccagca |
| | | atgacgatcaagtgggcatctggagcggccccgcccctcagtgcattat |
| | | acctaacaaatgcacgcctccaaatgtggaaaatggaatattggtatct |
| | | gacaacagaagcttattttccttaaatgaagttgtggagtttaggtgtc |
| | | agcctggctttgtcatgaaaggaccccgccgtgtgaagtgccaggccct |
| | | gaacaaatgggagccggagctaccaagctgctccagggtatgtcagcca |
| | | cctccagatgtcctgcatgctgagcgtacccaaagggacaaggacaact |
| | | tttcacctgggcaggaagtgttctacagctgtgagcccggctacgacct |
| | | cagaggggctgcgtctatgcgctgcacaccccagggagactggagccct |
| | | gcagcccccacatgtgaagtgaaatcctgtgatgacttcatgggccaac |
| | | ttcttaatggccgtgtgctatttccagtaaatctccagcttggagcaaa |
| | | agtggattttgtttgtgatgaaggatttcaattaaaaggcagctctgct |
| | | agttactgtgtcttggctggaatggaaagcctttggaatagcagtgttc |
| | | cagtgtgtgaacaaatcttttgtccaagtcctccagttattcctaatgg |
| | | gagacacacaggaaaacctctggaagtctttccctttgggaaaacagta |
| | | aattacacatgcgacccccacccagacagagggacgagcttcgacctca |
| | | ttggagagagcaccatccgctgcacaagtgaccctcaagggaatggggt |
| | | ttggagcagccctgcccctcgctgtggaattctgggtcactgtcaagcc |
| | | ccagatcattttctgtttgccaagttgaaaacccaaaccaatgcatctg |
| | | actttcccattgggacatctttaaagtacgaatgccgtcctgagtacta |
| | | cgggaggccattctctatcacatgtctagataacctggtctggtcaagt |
| | | cccaaagatgtctgtaaacgtaaatcatgtaaaactcctccagatccag |
| | | tgaatggcatggtgcatgtgatcacagacatccaggttggatccagaat |
| | | caactattcttgtactacagggcaccgactcattggtcactcatctgct |
| | | gaatgtatcctctcgggcaatgctgcccattggagcacgaagccgccaa |
| | | tttgtcaacgaattccttgtgggctaccccccaccatcgccaatggaga |
| | | tttcattagcaccaacagagagaattttcactatggatcagtggtgacc |
| | | taccgctgcaatcctggaagcggagggagaaaggtgtttgagcttgtgg |
| | | gtgagccctccatatactgcaccagcaatgacgatcaagtgggcatctg |
| | | gagcggcccggcccctcagtgcattatacctaacaaatgcacgcctcca |
| | | aatgtggaaaatggaatattggtatctgacaacagaagcttattttcct |
| | | taaatgaagttgtggagtttaggtgtcagcctggctttgtcatgaaagg |
| | | accccgccgtgtgaagtgccaggccctgaacaaatgggagccggagcta |
| | | ccaagctgctccagggtatgtcagccacctccagatgtcctgcatgctg |
| | | agcgtacccaaagggacaaggacaacttttcacccgggcaggaagtgtt |
| | | ctacagctgtgagcccggctacgacctcagaggggctgcgtctatgcgc |
| | | tgcacaccccagggagactggagccctgcagcccccacatgtgaagtga |
| | | aatcctgtgatgacttcatgggccaacttcttaatggccgtgtgctatt |
| | | tccagtaaatctccagcttggagcaaaagtggattttgtttgtgatgaa |
| | | ggatttcaattaaaaggcagctctgctagttactgtgtcttggctggaa |
| | | tggaaagcctttggaatagcagtgttccagtgtgtgaacaaatcttttg |
| | | tccaagtcctccagttattcctaatgggagacacacaggaaaacctctg |
| | | gaagtctttccctttgggaaaacagtaaattacacatgcgacccccacc |
| | | cagacagagggacgagcttcgacctcattggagagagcaccatccgctg |
| | | cacaagtgaccctcaagggaatggggtttggagcagccctgcccctcgc |
| | | tgtggaattctgggtcactgtcaagccccagatcattttctgtttgcca |
| | | agttgaaaacccaaaccaatgcatctgactttcccattgggacatcttt |
| | | aaagtacgaatgccgtcctgagtactacgggaggccattctctatcaca |
| | | tgtctagataacctggtctggtcaagtcccaaagatgtctgtaaacgta |
| | | aatcatgtaaaactcctccagatccagtgaatggcatggtgcatgtgat |
| | | cacagacatccaggttggatccagaatcaactattcttgtactacaggg |
| | | caccgactcattggtcactcatctgctgaatgtatcctctcgggcaatg |
| | | ctgcccattggagcacgaagccgccaatttgtcaacgaattccttgtgg |
| | | gctaccccccaccatcgccaatggagatttcattagcaccaacagagag |
| | | aattttcactatggatcagtggtgacctaccgctgcaatcctggaagcg |
| | | gagggagaaaggtgtttgagcttgtgggtgagccctccatatactgcac |
| | | cagcaatgacgatcaagtgggcatctggagcggcccggcccctcagtgc |
| | | attatacctaacaaatgcacgcctccaaatgtggaaaatggaatattgg |
| | | tatctgacaacagaagcttattttccttaaatgaagttgtggagtttag |
| | | gtgtcagcctggctttgtcatgaaaggaccccgccgtgtgaagtgccag |
| | | gccctgaacaaatgggagccggagctaccaagctgctccagggtatgtc |
| | | agccacctccagatgtcctgcatgctgagcgtacccaaagggacaagga |
| | | caacttttcacccgggcaggaagtgttctacagctgtgagcccggctat |
| | | gacctcagaggggctgcgtctatgcgctgcacaccccagggagactgga |
| | | gccctgcagcccccacatgtgaagtgaaatcctgtgatgacttcatggg |
| | | ccaacttcttaatggccgtgtgctatttccagtaaatctccagcttgga |
| | | gcaaaagtggattttgtttgtgatgaaggatttcaattaaaaggcagct |
| | | ctgctagttattgtgtcttggctggaatggaaagcctttggaatagcag |
| | | tgttccagtgtgtgaacaaatcttttgtccaagtcctccagttattcct |
| | | aatgggagacacacaggaaaacctctggaagtctttccctttggaaaag |
| | | cagtaaattacacatgcgacccccacccagacagagggacgagcttcga |
| | | cctcattggagagagcaccatccgctgcacaagtgaccctcaagggaat |
| | | ggggtttggagcagccctgcccctcgctgtggaattctgggtcactgtc |
| | | aagccccagatcattttctgtttgccaagttgaaaacccaaaccaatgc |
| | | atctgactttcccattgggacatctttaaagtacgaatgccgtcctgag |
| | | tactacgggaggccattctctatcacatgtctagataacctggtctggt |
| | | caagtcccaaagatgtctgtaaacgtaaatcatgtaaaactcctccaga |
| | | tccagtgaatggcatggtgcatgtgatcacagacatccaggttggatcc |
| | | agaatcaactattcttgtactacagggcaccgactcattggtcactcat |
| | | ctgctgaatgtatcctctcaggcaatactgcccattggagcacgaagcc |
| | | gccaatttgtcaacgaattccttgtgggctacccccaaccatcgccaat |
| | | ggagatttcattagcaccaacagagagaattttcactatggatcagtgg |
| | | tgacctaccgctgcaatcttggaagcagagggagaaaggtgtttgagct |
| | | tgtgggtgagccctccatatactgcaccagcaatgacgatcaagtgggc |
| | | atctggagcggccccgcccctcagtgcattata |
| 43 | A13L-CCPH₍₂₁₋₂₆₆₎ | atgattggtattcttttgttgatcggtatttgcgtagcagttaccgtcg |
| | (nt) | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaggaggaggtggatccttaagctgtcctacacgtaa |
| | | ccagtatgtttctgtcaaatatgtgaatctaactaactattcaggcccg |
| | | tatccaaacgggacaacgctacacgtgacatgccgtgaaggatatgcaa |
| | | aaagaccagtacaaactgttacatgcgtcaatggtaactggactgtacc |
| | | taaaaagtgtcagaaaaagaaatgttctacaccgcaagatcttttaaat |
| | | ggaagatatactgtaactggtaatttatattacggttcagttatcactt |
| | | atacttgtaattcaggctacagcttaattggaagcacaacatcagcttg |
| | | tttacttaaacgaggtggtcgtgttgactggactccacgacctccaatt |
| | | tgtgacattaaaaaatgtaaacctcctccacaaatagctaatgggactc |
| | | acactaatgtcaaagatttctatacttatttagatacagttacgtactc |
| | | atgcaatgacgaaacaaagttaactttaacaggcccttcatcgaaactt |
| | | tgttcagaaactggctcatgggtacctaatggagaaactaagtgtgaat |
| | | ttatattttgtaaactacctcaagttgcgaatgcgtacgttgaagttag |
| | | aaagtcagctacgagcatgcaatatttgcatataaatgttaaatgttat |
| | | aaaggatttatgctatatggagaaactcctaatacgtgtaaccatggag |
| | | tatggtctccagctattcctgaatgtatgaag |
| 44 | A13L-ORF4₍₂₂₋₂₆₈₎ | atgattggtattcttttgttgatcggtatttgcgtagcagttaccgtcg |
| | (nt) | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaggcggaggtggaagtatgtgccaccatcttcctca |
| | | aatgcccacactaacatctcctaaatttacaccaccagtcaaaagtggc |
| | | accaccctgcagctccgctgtaggccagggttcacaccaggcgcagggg |
| | | ggcggaagacagtaacctgcttaggggataatatgtggacaaccgtgac |
| | | tccctgtaacagaaagaggtgcccacacgttacctttccaaccaatggg |
| | | ggcgctaattaccattttaaagacaatgataccagtcctagctatggta |
| | | ccgaggcagtgttttactgtgatcccggctacaatcttctgggagaaag |
| | | taaattattttgtgagctgcaggataataataaagtgggctggagtggg |
| | | gaatctcccatatgtgacataaaaaagtgtgcacctcccgaagtcaagt |
| | | ccccagcctatatactgagggccaaagatgtgtataacacccgagaggt |
| | | gaccagatttagttgtcctataaatctgaagctatatgggccaacccac |
| | | gcagtctgtgagggacccgggtggacaccatccaccagtcccttttgcc |
| | | tggaaattccctgtggccaacctacaattccgcatgccacaattcaatc |
| | | taagccaggggctcccccaggctctagagaacttgttgtcaattgcgat |
| | | ccaggttacatgcccctagatgggaccaccttgacatgcgaaggcagag |
| | | gtgtttggaaactgccccttcctgaatgtgttgcc |
| 45 | A13L-x-CD55₍₃₅₋₂₈₄₎ | atgattggtattcttttgttgatcggtatttgcgtagcagttaccgtcg |
| | (nt) | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtagactgtggccttcccccagatgtacctaatgccca |
| | | gccagctttggaaggccgtacaagttttcccgaggatactgtaataacg |
| | | tacaaatgtgaagaaagctttgtgaaaattcctggcgagaaggactcag |
| | | tgatctgccttaagggcagtcaatggtcagatattgaagagttctgcaa |
| | | tcgtagctgcgaggtgccaacaaggctaaattctgcatccctcaaacag |
| | | ccttatatcactcagaattattttccagtcggtactgttgtggaatatg |
| | | agtgccgtccaggttacagaagagaaccttctctatcaccaaaactaac |
| | | ttgccttcagaatttaaaatggtccacagcagtcgaattttgtaaaaag |
| | | aaatcatgccctaatccgggagaaatacgaaatggtcagattgatgtac |
| | | caggtggcatattatttggtgcaaccatctccttctcatgtaacacagg |
| | | gtacaaattatttggctcgacttctagtttttgtcttatttcaggcagc |
| | | tctgtccagtggagtgacccgttgccagagtgcagagaaatttattgtc |
| | | cagcaccaccacaaattgacaatggaataattcaaggggaacgtgacca |
| | | ttatggatatagacagtctgtaacgtatgcatgtaataaaggattcacc |
| | | atgattggagagcactctatttattgtactgtgaataatgatgaaggag |
| | | agtggagtggcccaccacctgaatgcaga |
| 46 | A13L-CD55₍₃₅₋₂₈₄₎-V5 | atgattggtattcttttgttgatcggtatttgcgtagcagttaccgtcg |
| | (nt) | ccatcctatactcgatgtataataagatcaagaactcacaaaatccgaa |
| | | tccaagtccgaatttaaattcgcctcctccagaaccaaaaaataccaag |
| | | tttgtaaataatctggaaaaggatcatattagttcattgtataatctag |
| | | ttaaatcttctgtaggaggaggtggatccgactgtggccttcccccaga |
| | | tgtacctaatgcccagccagctttggaaggccgtacaagttttcccgag |
| | | gatactgtaataacgtacaaatgtgaagaaagctttgtgaaaattcctg |
| | | gcgagaaggactcagtgatctgccttaagggcagtcaatggtcagatat |
| | | tgaagagttctgcaatcgtagctgcgaggtgccaacaaggctaaattct |
| | | gcatccctcaaacagccttatatcactcagaattattttccagtcggta |
| | | ctgttgtggaatatgagtgccgtccaggttacagaagagaaccttctct |
| | | atcaccaaaactaacttgccttcagaatttaaaatggtccacagcagtc |
| | | gaattttgtaaaaagaaatcatgccctaatccgggagaaatacgaaatg |
| | | gtcagattgatgtaccaggtggcatattatttggtgcaaccatctcctt |
| | | ctcatgtaacacagggtacaaattatttggctcgacttctagtttttgt |
| | | cttatttcaggcagctctgtccagtggagtgacccgttgccagagtgca |
| | | gagaaatttattgtccagcaccaccacaaattgacaatggaataattca |
| | | aggggaacgtgaccattatggatatagacagtctgtaacgtatgcatgt |
| | | aataaaggattcaccatgattggagagcactctatttattgtactgtga |
| | | ataatgatgaaggagagtggagtggcccaccacctgaatgcagaggaac |
| | | cggtaagcctatccctaaccctctcctcggtctcgattctacg |
| 47 | A13-VCP (prt) | MIGILLLIGICVAVTVAILYSMYNKIKNSQNPNPSPNLNSPPPEPKNTK |
| | | FVNNLEKDHISSLYNLVKSSVGGGGSSCTIPSRPINMKFKNSVETDANA |
| | | NYNIGDTIEYLCLPGYRKQKMGPIYAKCTGTGWTLFNQCIKRRCPSPRD |
| | | IDNGQLDIGGVDFGSSITYSCNSGYHLIGESKSYCELGSTGSMVWNPEA |
| | | PICESVKCQSPPSISNGRHNGYEDFYTDGSVVTYSCNSGYSLIGNSGVL |
| | | CSGGEWSDPPTCQIVKCPHPTISNGYLSSGFKRSYSYNDNVDEKCKYGY |
| | | KLSGSSSSTCSPGNTWKPELPKCVR |
| 48 | A13-VCPx2 (prt) | MIGILLLIGICVAVTVAILYSMYNKIKNSQNPNPSPNLNSPPPEPKNTK |
| | | FVNNLEKDHISSLYNLVKSSVGGGGSSCTIPSRPINMKFKNSVETDANA |
| | | NYNIGDTIEYLCLPGYRKQKMGPIYAKCTGTGWTLENQCIKRRCPSPRD |
| | | IDNGQLDIGGVDFGSSITYSCNSGYHLIGESKSYCELGSTGSMVWNPEA |
| | | PICESVKCQSPPSISNGRHNGYEDFYTDGSVVTYSCNSGYSLIGNSGVL |
| | | CSGGEWSDPPTCQIVKCPHPTISNGYLSSGFKRSYSYNDNVDEKCKYGY |
| | | KLSGSSSSTCSPGNTWKPELPKCVRGGGGSSCTIPSRPINMKFKNSVET |
| | | DANANYNIGDTIEYLCLPGYRKQKMGPIYAKCTGTGWTLFNQCIKRRCP |
| | | SPRDIDNGOLDIGGVDFGSSITYSCNSGYHLIGESKSYCELGSTGSMVW |
| | | NPEAPICESVKCQSPPSISNGRHNGYEDFYTDGSVVTYSCNSGYSLIGN |
| | | SGVLCSGGEWSDPPTCQIVKCPHPTISNGYLSSGFKRSYSYNDNVDEKC |
| | | KYGYKLSGSSSSTCSPGNTWKPELPKCVR |
| 49 | A13-VCPx4 (prt) | MIGILLLIGICVAVTVAILYSMYNKIKNSQNPNPSPNLNSPPPEPKNTK |
| | | FVNNLEKDHISSLYNLVKSSVGGGGSSCTIPSRPINMKFKNSVETDANA |
| | | NYNIGDTIEYLCLPGYRKQKMGPIYAKCTGTGWTLFNQCIKRRCPSPRD |
| | | IDNGQLDIGGVDFGSSITYSCNSGYHLIGESKSYCELGSTGSMVWNPEA |
| | | PICESVKCQSPPSISNGRHNGYEDFYTDGSVVTYSCNSGYSLIGNSGVL |
| | | CSGGEWSDPPTCQIVKCPHPTISNGYLSSGFKRSYSYNDNVDFKCKYGY |
| | | KLSGSSSSTCSPGNTWKPELPKCVRGGGGSSCTIPSRPINMKFKNSVET |
| | | DANANYNIGDTIEYLCLPGYRKQKMGPIYAKCTGTGWTLENQCIKRRCP |
| | | SPRDIDNGOLDIGGVDFGSSITYSCNSGYHLIGESKSYCELGSTGSMVW |
| | | NPEAPICESVKCQSPPSISNGRHNGYEDFYTDGSVVTYSCNSGYSLIGN |
| | | SGVLCSGGEWSDPPTCQIVKCPHPTISNGYLSSGFKRSYSYNDNVDFKC |
| | | KYGYKLSGSSSSTCSPGNTWKPELPKCVRGGGGSSCTIPSRPINMKFKN |
| | | SVETDANANYNIGDTIEYLCLPGYRKQKMGPIYAKCTGTGWTLENQCIK |
| | | RRCPSPRDIDNGQLDIGGVDFGSSITYSCNSGYHLIGESKSYCELGSTG |
| | | SMVWNPEAPICESVKCQSPPSISNGRHNGYEDFYTDGSVVTYSCNSGYS |
| | | LIGNSGVLCSGGEWSDPPTCQIVKCPHPTISNGYLSSGFKRSYSYNDNV |
| | | DFKCKYGYKLSGSSSSTCSPGNTWKPELPKCVRGGGGSSCTIPSRPINM |
| | | KFKNSVETDANANYNIGDTIEYLCLPGYRKQKMGPIYAKCTGTGWTLFN |
| | | QCIKRRCPSPRDIDNGOLDIGGVDFGSSITYSCNSGYHLIGESKSYCEL |
| | | GSTGSMVWNPEAPICESVKCQSPPSISNGRHNGYEDFYTDGSVVTYSCN |
| | | SGYSLIGNSGVLCSGGEWSDPPTCQIVKCPHPTISNGYLSSGFKRSYSY |
| | | NDNVDFKCKYGYKLSGSSSSTCSPGNTWKPELPKCVR |
| 50 | A13-VCPmut (prt) | MIGILLLIGICVAVTVAILYSMYNKIKNSQNPNPSPNLNSPPPEPKNTK |
| | | FVNNLEKDHISSLYNLVKSSVGGGGSSCTIPSRPINMKFKNSVETDANA |
| | | NYNIGDTIEYLCLPGYRKQKMGPIYAKCTGTGWTLFNQCIKRRCPSPRD |
| | | IDNGQLDIGGVDFGSSITYSCNSGYHLIGESKSYCELGSTGSMVWNPEA |
| | | PICESVKCQSPPSISNGRHNGYEDFYTDGSVVTYSCNSGYSLIGNSGVL |
| | | CSGGEWSDPPTCQIVKCPHPTISNGYLSSGFKRSYSYNDNVDFKCKYGY |
| | | KLSGSSSSTCSPGNTWKPELPKCVR |
| 51 | A13-CD55₍₃₅₋₂₈₄₎ | MIGILLLIGICVAVTVAILYSMYNKIKNSQNPNPSPNLNSPPPEPKNTK |
| | (prt) | FVNNLEKDHISSLYNLVKSSVGGGGSDCGLPPDVPNAQPALEGRTSFPE |
| | | DTVITYKCEESFVKIPGEKDSVICLKGSQWSDIEEFCNRSCEVPTRLNS |
| | | ASLKQPYITQNYFPVGTVVEYECRPGYRREPSLSPKLTCLQNLKWSTAV |
| | | EFCKKKSCPNPGEIRNGQIDVPGGILFGATISFSCNTGYKLFGSTSSFC |
| | | LISGSSVQWSDPLPECREIYCPAPPQIDNGIIQGERDHYGYRQSVTYAC |
| | | NKGFTMIGEHSIYCTVNNDEGEWSGPPPECR |
| 52 | A13-CD35₍₄₂₋₁₅₈₄₎ | MIGILLLIGICVAVTVAILYSMYNKIKNSQNPNPSPNLNSPPPEPKNTK |
| | (prt) | FVNNLEKDHISSLYNLVKSSVGGGGSGGGGSGGGGSQCNAPEWLPFARP |
| | | TNLTDEFEFPIGTYLNYECRPGYSGRPFSIICLKNSVWTGAKDRCRRKS |
| | | CRNPPDPVNGMVHVIKGIQFGSQIKYSCTKGYRLIGSSSATCIISGDTV |
| | | IWDNETPICDRIPCGLPPTITNGDFISTNRENFHYGSVVTYRCNPGSGG |
| | | RKVFELVGEPSIYCTSNDDQVGIWSGPAPQCIIPNKCTPPNVENGILVS |
| | | DNRSLFSLNEVVEFRCQPGFVMKGPRRVKCQALNKWEPELPSCSRVCQP |
| | | PPDVLHAERTQRDKDNFSPGQEVFYSCEPGYDLRGAASMRCTPQGDWSP |
| | | AAPTCEVKSCDDFMGQLLNGRVLFPVNLQLGAKVDFVCDEGFQLKGSSA |
| | | SYCVLAGMESLWNSSVPVCEQIFCPSPPVIPNGRHTGKPLEVEPFGKTV |
| | | NYTCDPHPDRGTSEDLIGESTIRCTSDPQGNGVWSSPAPRCGILGHCQA |
| | | PDHFLFAKLKTQTNASDEPIGTSLKYECRPEYYGRPFSITCLDNLVWSS |
| | | PKDVCKRKSCKTPPDPVNGMVHVITDIQVGSRINYSCTTGHRLIGHSSA |
| | | ECILSGNAAHWSTKPPICQRIPCGLPPTIANGDFISTNRENFHYGSVVT |
| | | YRCNPGSGGRKVFELVGEPSIYCTSNDDQVGIWSGPAPQCIIPNKCTPP |
| | | NVENGILVSDNRSLESLNEVVEFRCQPGFVMKGPRRVKCQALNKWEPEL |
| | | PSCSRVCQPPPDVLHAERTQRDKDNESPGQEVFYSCEPGYDLRGAASMR |
| | | CTPQGDWSPAAPTCEVKSCDDEMGQLLNGRVLFPVNLQLGAKVDFVCDE |
| | | GFQLKGSSASYCVLAGMESLWNSSVPVCEQIFCPSPPVIPNGRHTGKPL |
| | | EVFPFGKTVNYTCDPHPDRGTSFDLIGESTIRCTSDPQGNGVWSSPAPR |
| | | CGILGHCQAPDHELFAKLKTQTNASDFPIGTSLKYECRPEYYGRPFSIT |
| | | CLDNLVWSSPKDVCKRKSCKTPPDPVNGMVHVITDIQVGSRINYSCTTG |
| | | HRLIGHSSAECILSGNAAHWSTKPPICQRIPCGLPPTIANGDFISTNRE |
| | | NFHYGSVVTYRCNPGSGGRKVFELVGEPSIYCTSNDDQVGIWSGPAPQC |
| | | IIPNKCTPPNVENGILVSDNRSLFSLNEVVEFRCQPGFVMKGPRRVKCQ |
| | | ALNKWEPELPSCSRVCQPPPDVLHAERTQRDKDNESPGQEVFYSCEPGY |
| | | DLRGAASMRCTPQGDWSPAAPTCEVKSCDDFMGQLLNGRVLFPVNLQLG |
| | | AKVDFVCDEGFQLKGSSASYCVLAGMESLWNSSVPVCEQIFCPSPPVIP |
| | | NGRHTGKPLEVEPFGKAVNYTCDPHPDRGTSEDLIGESTIRCTSDPQGN |
| | | GVWSSPAPRCGILGHCQAPDHELFAKLKTQTNASDEPIGTSLKYECRPE |
| | | YYGRPFSITCLDNLVWSSPKDVCKRKSCKTPPDPVNGMVHVITDIQVGS |
| | | RINYSCTTGHRLIGHSSAECILSGNTAHWSTKPPICQRIPCGLPPTIAN |
| | | GDFISTNRENFHYGSVVTYRCNLGSRGRKVFELVGEPSIYCTSNDDQVG |
| | | IWSGPAPQCII |
| 53 | A13-CCPH₍₂₁₋₂₆₆₎ | MIGILLLIGICVAVTVAILYSMYNKIKNSQNPNPSPNLNSPPPEPKNTK |
| | (prt) | FVNNLEKDHISSLYNLVKSSVGGGGSLSCPTRNQYVSVKYVNLTNYSGP |
| | | YPNGTTLHVTCREGYAKRPVQTVTCVNGNWTVPKKCQKKKCSTPQDLLN |
| | | GRYTVTGNLYYGSVITYTCNSGYSLIGSTTSACLLKRGGRVDWTPRPPI |
| | | CDIKKCKPPPQIANGTHTNVKDFYTYLDTVTYSCNDETKLTLTGPSSKL |
| | | CSETGSWVPNGETKCEFIFCKLPQVANAYVEVRKSATSMQYLHINVKCY |
| | | KGFMLYGETPNTCNHGVWSPAIPECMK |
| 54 | A13-ORF4₍₂₂₋₂₆₈₎ | MIGILLLIGICVAVTVAILYSMYNKIKNSQNPNPSPNLNSPPPEPKNTK |
| | (prt) | FVNNLEKDHISSLYNLVKSSVGGGGSMCHHLPQMPTLTSPKFTPPVKSG |
| | | TTLQLRCRPGFTPGAGGRKTVTCLGDNMWTTVTPCNRKRCPHVTFPTNG |
| | | GANYHFKDNDTSPSYGTEAVFYCDPGYNLLGESKLFCELQDNNKVGWSG |
| | | ESPICDIKKCAPPEVKSPAYILRAKDVYNTREVTRESCPINLKLYGPTH |
| | | AVCEGPGWTPSTSPFCLEIPCGQPTIPHATIQSKPGAPPGSRELVVNCD |
| | | PGYMPLDGTTLTCEGRGVWKLPLPECVA |
| 55 | A13L-x-CD55₍₃₅₋₂₈₄₎ | MIGILLLIGICVAVTVAILYSMYNKIKNSQNPNPSPNLNSPPPEPKNTK |
| | (prt) | FVNNLEKDHISSLYNLVKSSVDCGLPPDVPNAQPALEGRTSFPEDTVIT |
| | | YKCEESFVKIPGEKDSVICLKGSQWSDIEEFCNRSCEVPTRLNSASLKQ |
| | | PYITQNYFPVGTVVEYECRPGYRREPSLSPKLTCLQNLKWSTAVEFCKK |
| | | KSCPNPGEIRNGQIDVPGGILFGATISFSCNTGYKLFGSTSSFCLISGS |
| | | SVQWSDPLPECREIYCPAPPQIDNGIIQGERDHYGYRQSVTYACNKGFT |
| | | MIGEHSIYCTVNNDEGEWSGPPPECR |
| 56 | A13L-CD55₍₃₅₋₂₈₄₎-V5 | MIGILLLIGICVAVTVAILYSMYNKIKNSQNPNPSPNLNSPPPEPKNTK |
| | (prt) | FVNNLEKDHISSLYNLVKSSVGGGGSDCGLPPDVPNAQPALEGRTSFPE |
| | | DTVITYKCEESFVKIPGEKDSVICLKGSQWSDIEEFCNRSCEVPTRINS |
| | | ASLKQPYITQNYFPVGTVVEYECRPGYRREPSLSPKLTCLQNLKWSTAV |
| | | EFCKKKSCPNPGEIRNGQIDVPGGILFGATISFSCNTGYKLFGSTSSFC |
| | | LISGSSVQWSDPLPECREIYCPAPPQIDNGIIQGERDHYGYROSVTYAC |
| | | NKGFTMIGEHSIYCTVNNDEGEWSGPPPECRGTGKPIPNPLLGLDST |
| 57 | VCP-A27L (nt) | atgtcctgtactattccgtcacgacccattaatatgaaatttaagaata |
| | | gtgtggagactgatgctaatgctaattacaacataggagacactataga |
| | | atatctatgtctacctggatacagaaagcaaaaaatgggacctatatat |
| | | gctaaatgtacaggtactggatggacactctttaatcaatgtattaaac |
| | | ggagatgcccatcgccacgagatatcgataatggccaacttgatattgg |
| | | tggagtagactttggctctagtataacgtactcttgtaatagcggatat |
| | | catttgatcggtgaatctaaatcgtattgtgaattaggatctactggat |
| | | ctatggtatggaatcccgaggcacctatttgtgaatctgttaaatgcca |
| | | atcccctccatctatatccaacggaagacataacggatacgaggatttc |
| | | tacaccgatgggagcgttgtaacttatagttgcaatagtggatattcgt |
| | | tgattggtaactctggtgtcctgtgttcaggaggagaatggtccgatcc |
| | | acccacgtgtcagattgttaaatgtccacatcctacaatatcaaacgga |
| | | tacttgtctagcgggtttaaaagatcatactcatacaacgacaatgtag |
| | | actttaagtgcaagtacggatataaactatctggttcctcatcatctac |
| | | ttgctctccaggaaatacatggaagccggaacttccaaaatgtgtacgc |
| | | ggcggaggtggaagtggaggcggtggatccggtggaggaggcagcgacg |
| | | gaactcttttccccggagatgacgatcttgcaattccagcaactgaatt |
| | | tttttctacaaaggctgataaaaagccagaggctaaacgcgaagcaatt |
| | | gttaaagccgatgaagacgacaatgaggaaactctcaaacaacggctaa |
| | | ctaatttggaaaaaaagattactaatgtaacaacaaagtttgaacaaat |
| | | agaaaagtgttgtaaacgcaacgatgaagttctatttaggttggaaaat |
| | | cacgctgaaactctaagagcggctatgatatctctggctaaaaagattg |
| | | atgttcagactggacggcgtccatatgag |
| 58 | VCPmut-A27L (nt) | atgtcctgtactattccgtcacgacccattaatatgaaatttaagaata |
| | | gtgtggagactgatgctaatgctaattacaacataggagacactataga |
| | | atatctatgtctacctggatacagaaagcaaaaaatgggacctatatat |
| | | gctaaatgtacaggtactggatggacactctttaatcaatgtattaaac |
| | | ggagatgcccatcgccacgagatatcgataatggccaccttgatattgg |
| | | tggagtagactttggctctagtataacgtactcttgtaatagcggatat |
| | | catttgatcggtgaatctaaatcgtattgtgaattaggatctactggat |
| | | ctatggtatggaatcccaaggcacctatttgtgaatctgttaaatgcca |
| | | atcccctccatctatatccaacggaagacataacggatacgaggatttc |
| | | tacaccgatgggagcgttgtaacttatagttgcaatagtggatattcgt |
| | | tgattggtaactctggtgtcctgtgttcaggaggagaatggtccgatcc |
| | | acccacgtgtcagattgttaaatgtccacatcctacaatatcaaacgga |
| | | tacttgtctagcgggtttaaaagatcatactcatacaacgacaatgtag |
| | | actttaagtgcaagtacggatataaactatctggttcctcatcatctac |
| | | ttgctctccaggaaatacatggaagccggaacttccaaaatgtgtacgc |
| | | ggcggaggtggaagtggaggcggtggatccggtggaggaggcagcgacg |
| | | gaactcttttccccggagatgacgatcttgcaattccagcaactgaatt |
| | | tttttctacaaaggctgataaaaagccagaggctaaacgcgaagcaatt |
| | | gttaaagccgatgaagacgacaatgaggaaactctcaaacaacggctaa |
| | | ctaatttggaaaaaaagattactaatgtaacaacaaagtttgaacaaat |
| | | agaaaagtgttgtaaacgcaacgatgaagttctatttaggttggaaaat |
| | | cacgctgaaactctaagagcggctatgatatctctggctaaaaagattg |
| | | atgttcagactggacggcgtccatatgag |
| 59 | CD55₍₃₅₋₂₈₄₎-A27L | atggactgtggccttcccccagatgtacctaatgcccagccagctttgg |
| | (nt) | aaggccgtacaagttttcccgaggatactgtaataacgtacaaatgtga |
| | | agaaagctttgtgaaaattcctggcgagaaggactcagtgatctgcctt |
| | | aagggcagtcaatggtcagatattgaagagttctgcaatcgtagctgcg |
| | | aggtgccaacaaggctaaattctgcatccctcaaacagccttatatcac |
| | | tcagaattattttccagtcggtactgttgtggaatatgagtgccgtcca |
| | | ggttacagaagagaaccttctctatcaccaaaactaacttgccttcaga |
| | | atttaaaatggtccacagcagtcgaattttgtaaaaagaaatcatgccc |
| | | taatccgggagaaatacgaaatggtcagattgatgtaccaggtggcata |
| | | ttatttggtgcaaccatctccttctcatgtaacacagggtacaaattat |
| | | ttggctcgacttctagtttttgtcttatttcaggcagctctgtccagtg |
| | | gagtgacccgttgccagagtgcagagaaatttattgtccagcaccacca |
| | | caaattgacaatggaataattcaaggggaacgtgaccattatggatata |
| | | gacagtctgtaacgtatgcatgtaataaaggattcaccatgattggaga |
| | | gcactctatttattgtactgtgaataatgatgaaggagagtggagtggc |
| | | ccaccacctgaatgcagaggcggaggtggaagtggaggcggtggatccg |
| | | gtggaggaggcagcgacggaactcttttccccggagatgacgatcttgc |
| | | aattccagcaactgaatttttttctacaaaggctgataaaaagccagag |
| | | gctaaacgcgaagcaattgttaaagccgatgaagacgacaatgaggaaa |
| | | ctctcaaacaacggctaactaatttggaaaaaaagattactaatgtaac |
| | | aacaaagtttgaacaaatagaaaagtgttgtaaacgcaacgatgaagtt |
| | | ctatttaggttggaaaatcacgctgaaactctaagagcggctatgatat |
| | | ctctggctaaaaagattgatgttcagactggacggcgtccatatgag |
| 60 | CD35₍₄₂₋₁₅₈₄₎-A27L | atgcaatgcaatgccccagaatggcttccatttgccaggcctaccaacc |
| | (nt) | taactgatgaatttgagtttcccattgggacatatctgaactatgaatg |
| | | ccgccctggttattccggaagaccgttttctatcatctgcctaaaaaac |
| | | tcagtctggactggtgctaaggacaggtgcagacgtaaatcatgtcgta |
| | | atcctccagatcctgtgaatggcatggtgcatgtgatcaaaggcatcca |
| | | gttcggatcccaaattaaatattcttgtactaaaggataccgactcatt |
| | | ggttcctcgtctgccacatgcatcatctcaggtgatactgtcatttggg |
| | | ataatgaaacacctatttgtgacagaattccttgtgggctaccccccac |
| | | catcaccaatggagatttcattagcaccaacagagagaattttcactat |
| | | ggatcagtggtgacctaccgctgcaatcctggaagcggagggagaaagg |
| | | tgtttgagcttgtgggtgagccctccatatactgcaccagcaatgacga |
| | | tcaagtgggcatctggagcggccccgcccctcagtgcattatacctaac |
| | | aaatgcacgcctccaaatgtggaaaatggaatattggtatctgacaaca |
| | | gaagcttattttccttaaatgaagttgtggagtttaggtgtcagcctgg |
| | | ctttgtcatgaaaggaccccgccgtgtgaagtgccaggccctgaacaaa |
| | | tgggagccggagctaccaagctgctccagggtatgtcagccacctccag |
| | | atgtcctgcatgctgagcgtacccaaagggacaaggacaacttttcacc |
| | | tgggcaggaagtgttctacagctgtgagcccggctacgacctcagaggg |
| | | gctgcgtctatgcgctgcacaccccagggagactggagccctgcagccc |
| | | ccacatgtgaagtgaaatcctgtgatgacttcatgggccaacttcttaa |
| | | tggccgtgtgctatttccagtaaatctccagcttggagcaaaagtggat |
| | | tttgtttgtgatgaaggatttcaattaaaaggcagctctgctagttact |
| | | gtgtcttggctggaatggaaagcctttggaatagcagtgttccagtgtg |
| | | tgaacaaatcttttgtccaagtcctccagttattcctaatgggagacac |
| | | acaggaaaacctctggaagtctttccctttgggaaaacagtaaattaca |
| | | catgcgacccccacccagacagagggacgagcttcgacctcattggaga |
| | | gagcaccatccgctgcacaagtgaccctcaagggaatggggtttggagc |
| | | agccctgcccctcgctgtggaattctgggtcactgtcaagccccagatc |
| | | attttctgtttgccaagttgaaaacccaaaccaatgcatctgactttcc |
| | | cattgggacatctttaaagtacgaatgccgtcctgagtactacgggagg |
| | | ccattctctatcacatgtctagataacctggtctggtcaagtcccaaag |
| | | atgtctgtaaacgtaaatcatgtaaaactcctccagatccagtgaatgg |
| | | catggtgcatgtgatcacagacatccaggttggatccagaatcaactat |
| | | tcttgtactacagggcaccgactcattggtcactcatctgctgaatgta |
| | | tcctctcgggcaatgctgcccattggagcacgaagccgccaatttgtca |
| | | acgaattccttgtgggctaccccccaccatcgccaatggagatttcatt |
| | | agcaccaacagagagaattttcactatggatcagtggtgacctaccgct |
| | | gcaatcctggaagcggagggagaaaggtgtttgagcttgtgggtgagcc |
| | | ctccatatactgcaccagcaatgacgatcaagtgggcatctggagcggc |
| | | ccggcccctcagtgcattatacctaacaaatgcacgcctccaaatgtgg |
| | | aaaatggaatattggtatctgacaacagaagcttattttccttaaatga |
| | | agttgtggagtttaggtgtcagcctggctttgtcatgaaaggaccccgc |
| | | cgtgtgaagtgccaggccctgaacaaatgggagccggagctaccaagct |
| | | gctccagggtatgtcagccacctccagatgtcctgcatgctgagcgtac |
| | | ccaaagggacaaggacaacttttcacccgggcaggaagtgttctacagc |
| | | tgtgagcccggctacgacctcagaggggctgcgtctatgcgctgcacac |
| | | cccagggagactggagccctgcagcccccacatgtgaagtgaaatcctg |
| | | tgatgacttcatgggccaacttcttaatggccgtgtgctatttccagta |
| | | aatctccagcttggagcaaaagtggattttgtttgtgatgaaggatttc |
| | | aattaaaaggcagctctgctagttactgtgtcttggctggaatggaaag |
| | | cctttggaatagcagtgttccagtgtgtgaacaaatcttttgtccaagt |
| | | cctccagttattcctaatgggagacacacaggaaaacctctggaagtct |
| | | ttccctttgggaaaacagtaaattacacatgcgacccccacccagacag |
| | | agggacgagcttcgacctcattggagagagcaccatccgctgcacaagt |
| | | gaccctcaagggaatggggtttggagcagccctgcccctcgctgtggaa |
| | | ttctgggtcactgtcaagccccagatcattttctgtttgccaagttgaa |
| | | aacccaaaccaatgcatctgactttcccattgggacatctttaaagtac |
| | | gaatgccgtcctgagtactacgggaggccattctctatcacatgtctag |
| | | ataacctggtctggtcaagtcccaaagatgtctgtaaacgtaaatcatg |
| | | taaaactcctccagatccagtgaatggcatggtgcatgtgatcacagac |
| | | atccaggttggatccagaatcaactattcttgtactacagggcaccgac |
| | | tcattggtcactcatctgctgaatgtatcctctcgggcaatgctgccca |
| | | ttggagcacgaagccgccaatttgtcaacgaattccttgtgggctaccc |
| | | cccaccatcgccaatggagatttcattagcaccaacagagagaattttc |
| | | actatggatcagtggtgacctaccgctgcaatcctggaagcggagggag |
| | | aaaggtgtttgagcttgtgggtgagccctccatatactgcaccagcaat |
| | | gacgatcaagtgggcatctggagcggcccggcccctcagtgcattatac |
| | | ctaacaaatgcacgcctccaaatgtggaaaatggaatattggtatctga |
| | | caacagaagcttattttccttaaatgaagttgtggagtttaggtgtcag |
| | | cctggctttgtcatgaaaggaccccgccgtgtgaagtgccaggccctga |
| | | acaaatgggagccggagctaccaagctgctccagggtatgtcagccacc |
| | | tccagatgtcctgcatgctgagcgtacccaaagggacaaggacaacttt |
| | | tcacccgggcaggaagtgttctacagctgtgagcccggctatgacctca |
| | | gaggggctgcgtctatgcgctgcacaccccagggagactggagccctgc |
| | | agcccccacatgtgaagtgaaatcctgtgatgacttcatgggccaactt |
| | | cttaatggccgtgtgctatttccagtaaatctccagcttggagcaaaag |
| | | tggattttgtttgtgatgaaggatttcaattaaaaggcagctctgctag |
| | | ttattgtgtcttggctggaatggaaagcctttggaatagcagtgttcca |
| | | gtgtgtgaacaaatcttttgtccaagtcctccagttattcctaatggga |
| | | gacacacaggaaaacctctggaagtctttccctttggaaaagcagtaaa |
| | | ttacacatgcgacccccacccagacagagggacgagcttcgacctcatt |
| | | ggagagagcaccatccgctgcacaagtgaccctcaagggaatggggttt |
| | | ggagcagccctgcccctcgctgtggaattctgggtcactgtcaagcccc |
| | | agatcattttctgtttgccaagttgaaaacccaaaccaatgcatctgac |
| | | tttcccattgggacatctttaaagtacgaatgccgtcctgagtactacg |
| | | ggaggccattctctatcacatgtctagataacctggtctggtcaagtcc |
| | | caaagatgtctgtaaacgtaaatcatgtaaaactcctccagatccagtg |
| | | aatggcatggtgcatgtgatcacagacatccaggttggatccagaatca |
| | | actattcttgtactacagggcaccgactcattggtcactcatctgctga |
| | | atgtatcctctcaggcaatactgcccattggagcacgaagccgccaatt |
| | | tgtcaacgaattccttgtgggctacccccaaccatcgccaatggagatt |
| | | tcattagcaccaacagagagaattttcactatggatcagtggtgaccta |
| | | ccgctgcaatcttggaagcagagggagaaaggtgtttgagcttgtgggt |
| | | gagccctccatatactgcaccagcaatgacgatcaagtgggcatctgga |
| | | gcggccccgcccctcagtgcattataggcggaggtggaagtggaggcgg |
| | | tggatccggtggaggaggcagcgacggaactcttttccccggagatgac |
| | | gatcttgcaattccagcaactgaatttttttctacaaaggctgataaaa |
| | | agccagaggctaaacgcgaagcaattgttaaagccgatgaagacgacaa |
| | | tgaggaaactctcaaacaacggctaactaatttggaaaaaaagattact |
| | | aatgtaacaacaaagtttgaacaaatagaaaagtgttgtaaacgcaacg |
| | | atgaagttctatttaggttggaaaatcacgctgaaactctaagagcggc |
| | | tatgatatctctggctaaaaagattgatgttcagactggacggcgtcca |
| | | tatgag |
| 61 | CCPH₍₂₁₋₂₆₆₎-A27L | atgttaagctgtcctacacgtaaccagtatgtttctgtcaaatatgtga |
| | (nt) | atctaactaactattcaggcccgtatccaaacgggacaacgctacacgt |
| | | gacatgccgtgaaggatatgcaaaaagaccagtacaaactgttacatgc |
| | | gtcaatggtaactggactgtacctaaaaagtgtcagaaaaagaaatgtt |
| | | ctacaccgcaagatcttttaaatggaagatatactgtaactggtaattt |
| | | atattacggttcagttatcacttatacttgtaattcaggctacagctta |
| | | attggaagcacaacatcagcttgtttacttaaacgaggtggtcgtgttg |
| | | actggactccacgacctccaatttgtgacattaaaaaatgtaaacctcc |
| | | tccacaaatagctaatgggactcacactaatgtcaaagatttctatact |
| | | tatttagatacagttacgtactcatgcaatgacgaaacaaagttaactt |
| | | taacaggcccttcatcgaaactttgttcagaaactggctcatgggtacc |
| | | taatggagaaactaagtgtgaatttatattttgtaaactacctcaagtt |
| | | gcgaatgcgtacgttgaagttagaaagtcagctacgagcatgcaatatt |
| | | tgcatataaatgttaaatgttataaaggatttatgctatatggagaaac |
| | | tcctaatacgtgtaaccatggagtatggtctccagctattcctgaatgt |
| | | atgaagggcggaggtggaagtggaggcggtggatccggtggaggaggca |
| | | gcgacggaactcttttccccggagatgacgatcttgcaattccagcaac |
| | | tgaatttttttctacaaaggctgataaaaagccagaggctaaacgcgaa |
| | | gcaattgttaaagccgatgaagacgacaatgaggaaactctcaaacaac |
| | | ggctaactaatttggaaaaaaagattactaatgtaacaacaaagtttga |
| | | acaaatagaaaagtgttgtaaacgcaacgatgaagttctatttaggttg |
| | | gaaaatcacgctgaaactctaagagcggctatgatatctctggctaaaa |
| | | agattgatgttcagactggacggcgtccatatgag |
| 62 | ORF4₍₂₂₋₂₆₈₎-A27L | atgtgccaccatcttcctcaaatgcccacactaacatctcctaaattta |
| | (nt) | caccaccagtcaaaagtggcaccaccctgcagctccgctgtaggccagg |
| | | gttcacaccaggcgcaggggggcggaagacagtaacctgcttaggggat |
| | | aatatgtggacaaccgtgactccctgtaacagaaagaggtgcccacacg |
| | | ttacctttccaaccaatgggggcgctaattaccattttaaagacaatga |
| | | taccagtcctagctatggtaccgaggcagtgttttactgtgatcccggc |
| | | tacaatcttctgggagaaagtaaattattttgtgagctgcaggataata |
| | | ataaagtgggctggagtggggaatctcccatatgtgacataaaaaagtg |
| | | tgcacctcccgaagtcaagtccccagcctatatactgagggccaaagat |
| | | gtgtataacacccgagaggtgaccagatttagttgtcctataaatctga |
| | | agctatatgggccaacccacgcagtctgtgagggacccgggtggacacc |
| | | atccaccagtcccttttgcctggaaattccctgtggccaacctacaatt |
| | | ccgcatgccacaattcaatctaagccaggggctcccccaggctctagag |
| | | aacttgttgtcaattgcgatccaggttacatgcccctagatgggaccac |
| | | cttgacatgcgaaggcagaggtgtttggaaactgccccttcctgaatgt |
| | | gttgccggcggaggtggaagtggaggcggtggatccggtggaggaggca |
| | | gcgacggaactcttttccccggagatgacgatcttgcaattccagcaac |
| | | tgaatttttttctacaaaggctgataaaaagccagaggctaaacgcgaa |
| | | gcaattgttaaagccgatgaagacgacaatgaggaaactctcaaacaac |
| | | ggctaactaatttggaaaaaaagattactaatgtaacaacaaagtttga |
| | | acaaatagaaaagtgttgtaaacgcaacgatgaagttctatttaggttg |
| | | gaaaatcacgctgaaactctaagagcggctatgatatctctggctaaaa |
| | | agattgatgttcagactggacggcgtccatatgag |
| 63 | VCP-A27L (prt) | MSCTIPSRPINMKFKNSVETDANANYNIGDTIEYLCLPGYRKQKMGPIY |
| | | AKCTGTGWTLFNQCIKRRCPSPRDIDNGQLDIGGVDEGSSITYSCNSGY |
| | | HLIGESKSYCELGSTGSMVWNPEAPICESVKCQSPPSISNGRHNGYEDF |
| | | YTDGSVVTYSCNSGYSLIGNSGVLCSGGEWSDPPTCQIVKCPHPTISNG |
| | | YLSSGFKRSYSYNDNVDFKCKYGYKLSGSSSSTCSPGNTWKPELPKCVR |
| | | GGGGSGGGGSGGGGSDGTLFPGDDDLAIPATEFFSTKADKKPEAKREAI |
| | | VKADEDDNEETLKQRLTNLEKKITNVTTKFEQIEKCCKRNDEVLFRLEN |
| | | HAETLRAAMISLAKKIDVQTGRRPYE |
| 64 | VCPmut-A27L | MSCTIPSRPINMKFKNSVETDANANYNIGDTIEYLCLPGYRKQKMGPIY |
| | (prt) | AKCTGTGWTLFNQCIKRRCPSPRDIDNGHLDIGGVDEGSSITYSCNSGY |
| | | HLIGESKSYCELGSTGSMVWNPKAPICESVKCQSPPSISNGRHNGYEDF |
| | | YTDGSVVTYSCNSGYSLIGNSGVLCSGGEWSDPPTCQIVKCPHPTISNG |
| | | YLSSGFKRSYSYNDNVDFKCKYGYKLSGSSSSTCSPGNTWKPELPKCVR |
| | | GGGGSGGGGSGGGGSDGTLFPGDDDLAIPATEFFSTKADKKPEAKREAI |
| | | VKADEDDNEETLKQRLTNLEKKITNVTTKFEQIEKCCKRNDEVLFRLEN |
| | | HAETLRAAMISLAKKIDVQTGRRPYE |
| 65 | CD55₍₃₅₋₂₈₄₎-A27L | MDCGLPPDVPNAQPALEGRTSFPEDTVITYKCEESFVKIPGEKDSVICL |
| | (prt) | KGSQWSDIEEFCNRSCEVPTRLNSASLKQPYITQNYFPVGTVVEYECRP |
| | | GYRREPSLSPKLTCLQNLKWSTAVEFCKKKSCPNPGEIRNGQIDVPGGI |
| | | LFGATISFSCNTGYKLFGSTSSFCLISGSSVOWSDPLPECREIYCPAPP |
| | | QIDNGIIQGERDHYGYRQSVTYACNKGFTMIGEHSIYCTVNNDEGEWSG |
| | | PPPECRGGGGSGGGGSGGGGSDGTLFPGDDDLAIPATEFFSTKADKKPE |
| | | AKREAIVKADEDDNEETLKQRLTNLEKKITNVTTKFEQIEKCCKRNDEV |
| | | LFRLENHAETLRAAMISLAKKIDVQTGRRPYE |
| 66 | CD35₍₄₂₋₁₅₈₄₎-A27L | MQCNAPEWLPFARPTNLTDEFEFPIGTYLNYECRPGYSGRPFSIICLKN |
| | (prt) | SVWTGAKDRCRRKSCRNPPDPVNGMVHVIKGIQFGSQIKYSCTKGYRLI |
| | | GSSSATCIISGDTVIWDNETPICDRIPCGLPPTITNGDFISTNRENFHY |
| | | GSVVTYRCNPGSGGRKVFELVGEPSIYCTSNDDQVGIWSGPAPQCIIPN |
| | | KCTPPNVENGILVSDNRSLESLNEVVEFRCQPGFVMKGPRRVKCQALNK |
| | | WEPELPSCSRVCQPPPDVLHAERTQRDKDNESPGQEVFYSCEPGYDLRG |
| | | AASMRCTPQGDWSPAAPTCEVKSCDDFMGQLLNGRVLFPVNLOLGAKVD |
| | | FVCDEGFQLKGSSASYCVLAGMESLWNSSVPVCEQIFCPSPPVIPNGRH |
| | | TGKPLEVFPFGKTVNYTCDPHPDRGTSFDLIGESTIRCTSDPQGNGVWS |
| | | SPAPRCGILGHCQAPDHELFAKLKTQTNASDEPIGTSLKYECRPEYYGR |
| | | PFSITCLDNLVWSSPKDVCKRKSCKTPPDPVNGMVHVITDIQVGSRINY |
| | | SCTTGHRLIGHSSAECILSGNAAHWSTKPPICQRIPCGLPPTIANGDFI |
| | | STNRENFHYGSVVTYRCNPGSGGRKVFELVGEPSIYCTSNDDQVGIWSG |
| | | PAPQCIIPNKCTPPNVENGILVSDNRSLESLNEVVEFRCQPGFVMKGPR |
| | | RVKCQALNKWEPELPSCSRVCQPPPDVLHAERTQRDKDNESPGQEVFYS |
| | | CEPGYDLRGAASMRCTPQGDWSPAAPTCEVKSCDDFMGQLLNGRVLFPV |
| | | NLQLGAKVDFVCDEGFQLKGSSASYCVLAGMESLWNSSVPVCEQIFCPS |
| | | PPVIPNGRHTGKPLEVFPFGKTVNYTCDPHPDRGTSEDLIGESTIRCTS |
| | | DPQGNGVWSSPAPRCGILGHCQAPDHELFAKLKTQTNASDEPIGTSLKY |
| | | ECRPEYYGRPFSITCLDNLVWSSPKDVCKRKSCKTPPDPVNGMVHVITD |
| | | IQVGSRINYSCTTGHRLIGHSSAECILSGNAAHWSTKPPICQRIPCGLP |
| | | PTIANGDFISTNRENFHYGSVVTYRCNPGSGGRKVFELVGEPSIYCTSN |
| | | DDQVGIWSGPAPQCIIPNKCTPPNVENGILVSDNRSLESLNEVVEFRCQ |
| | | PGFVMKGPRRVKCQALNKWEPELPSCSRVCQPPPDVLHAERTQRDKDNF |
| | | SPGQEVFYSCEPGYDLRGAASMRCTPQGDWSPAAPTCEVKSCDDEMGQL |
| | | LNGRVLFPVNLQLGAKVDFVCDEGFQLKGSSASYCVLAGMESLWNSSVP |
| | | VCEQIFCPSPPVIPNGRHTGKPLEVFPFGKAVNYTCDPHPDRGTSEDLI |
| | | GESTIRCTSDPQGNGVWSSPAPRCGILGHCQAPDHELFAKLKTQTNASD |
| | | FPIGTSLKYECRPEYYGRPFSITCLDNLVWSSPKDVCKRKSCKTPPDPV |
| | | NGMVHVITDIQVGSRINYSCTTGHRLIGHSSAECILSGNTAHWSTKPPI |
| | | CQRIPCGLPPTIANGDFISTNRENFHYGSVVTYRCNLGSRGRKVFELVG |
| | | EPSIYCTSNDDQVGIWSGPAPQCIIGGGGSGGGGSGGGGSDGTLFPGDD |
| | | DLAIPATEFESTKADKKPEAKREAIVKADEDDNEETLKQRLTNLEKKIT |
| | | NVTTKFEQIEKCCKRNDEVLERLENHAETLRAAMISLAKKIDVQTGRRP |
| | | YE |
| 67 | CCPH₍₂₁₋₂₆₆₎-A27L | MLSCPTRNQYVSVKYVNLTNYSGPYPNGTTLHVTCREGYAKRPVQTVTC |
| | (prt) | VNGNWTVPKKCQKKKCSTPQDLLNGRYTVTGNLYYGSVITYTCNSGYSL |
| | | IGSTTSACLLKRGGRVDWTPRPPICDIKKCKPPPQIANGTHTNVKDFYT |
| | | YLDTVTYSCNDETKLTLTGPSSKLCSETGSWVPNGETKCEFIFCKLPQV |
| | | ANAYVEVRKSATSMQYLHINVKCYKGFMLYGETPNTCNHGVWSPAIPEC |
| | | MKGGGGSGGGGSGGGGSDGTLFPGDDDLAIPATEFFSTKADKKPEAKRE |
| | | AIVKADEDDNEETLKQRLTNLEKKITNVTTKFEQIEKCCKRNDEVLERL |
| | | ENHAETLRAAMISLAKKIDVQTGRRPYE |
| 68 | ORF4₍₂₂₋₂₆₈₎-A27L | MCHHLPQMPTLTSPKFTPPVKSGTTLQLRCRPGFTPGAGGRKTVTCLGD |
| | (prt) | NMWTTVTPCNRKRCPHVTFPTNGGANYHFKDNDTSPSYGTEAVFYCDPG |
| | | YNLLGESKLFCELQDNNKVGWSGESPICDIKKCAPPEVKSPAYILRAKD |
| | | VYNTREVTRFSCPINLKLYGPTHAVCEGPGWTPSTSPFCLEIPCGQPTI |
| | | PHATIQSKPGAPPGSRELVVNCDPGYMPLDGTTLTCEGRGVWKLPLPEC |
| | | VAGGGGSGGGGSGGGGSDGTLFPGDDDLAIPATEFFSTKADKKPEAKRE |
| | | AIVKADEDDNEETLKQRLTNLEKKITNVTTKFEQIEKCCKRNDEVLERL |
| | | ENHAETLRAAMISLAKKIDVQTGRRPYE |
| 69 | V5 tag (nt) | ggtaagcctatccctaaccctctcctcggtctcgattctacg |
| 70 | V5 tag (prt) | GKPIPNPLLGLDST |
| 71 | CD55₍₃₅₋₂₈₄₎ | DCGLPPDVPNAQPALEGRTSFPEDTVITYKCEESFVKIPGEKDSVICLK |
| | | GSQWSDIEEFCNRSCEVPTRLNSASLKQPYITQNYFPVGTVVEYECRPG |
| | | YRREPSLSPKLTCLQNLKWSTAVEFCKKKSCPNPGEIRNGQIDVPGGIL |
| | | FGATISFSCNTGYKLFGSTSSFCLISGSSVOWSDPLPECREIYCPAPPQ |
| | | IDNGIIQGERDHYGYRQSVTYACNKGFTMIGEHSIYCTVNNDEGEWSGP |
| | | PPECR |
| 72 | CD35₍₄₂₋₁₅₈₄₎ | QCNAPEWLPFARPTNLTDEFEFPIGTYLNYECRPGYSGRPESIICLKNS |
| | (prt) | VWTGAKDRCRRKSCRNPPDPVNGMVHVIKGIQFGSQIKYSCTKGYRLIG |
| | | SSSATCIISGDTVIWDNETPICDRIPCGLPPTITNGDFISTNRENFHYG |
| | | SVVTYRCNPGSGGRKVFELVGEPSIYCTSNDDQVGIWSGPAPQCIIPNK |
| | | CTPPNVENGILVSDNRSLESLNEVVEFRCQPGFVMKGPRRVKCQALNKW |
| | | EPELPSCSRVCQPPPDVLHAERTQRDKDNESPGQEVFYSCEPGYDLRGA |
| | | ASMRCTPQGDWSPAAPTCEVKSCDDEMGQLLNGRVLFPVNLQLGAKVDE |
| | | VCDEGFQLKGSSASYCVLAGMESLWNSSVPVCEQIFCPSPPVIPNGRHT |
| | | GKPLEVFPFGKTVNYTCDPHPDRGTSFDLIGESTIRCTSDPQGNGVWSS |
| | | PAPRCGILGHCQAPDHELFAKLKTQTNASDEPIGTSLKYECRPEYYGRP |
| | | FSITCLDNLVWSSPKDVCKRKSCKTPPDPVNGMVHVITDIQVGSRINYS |
| | | CTTGHRLIGHSSAECILSGNAAHWSTKPPICQRIPCGLPPTIANGDFIS |
| | | TNRENFHYGSVVTYRCNPGSGGRKVFELVGEPSIYCTSNDDQVGIWSGP |
| | | APQCIIPNKCTPPNVENGILVSDNRSLESLNEVVEFRCQPGFVMKGPRR |
| | | VKCQALNKWEPELPSCSRVCQPPPDVLHAERTQRDKDNESPGQEVFYSC |
| | | EPGYDLRGAASMRCTPQGDWSPAAPTCEVKSCDDEMGQLLNGRVLFPVN |
| | | LQLGAKVDFVCDEGFQLKGSSASYCVLAGMESLWNSSVPVCEQIFCPSP |
| | | PVIPNGRHTGKPLEVFPFGKTVNYTCDPHPDRGTSEDLIGESTIRCTSD |
| | | PQGNGVWSSPAPRCGILGHCQAPDHFLFAKLKTQTNASDFPIGTSLKYE |
| | | CRPEYYGRPFSITCLDNLVWSSPKDVCKRKSCKTPPDPVNGMVHVITDI |
| | | QVGSRINYSCTTGHRLIGHSSAECILSGNAAHWSTKPPICQRIPCGLPP |
| | | TIANGDFISTNRENFHYGSVVTYRCNPGSGGRKVFELVGEPSIYCTSND |
| | | DQVGIWSGPAPQCIIPNKCTPPNVENGILVSDNRSLESLNEVVEFRCQP |
| | | GFVMKGPRRVKCQALNKWEPELPSCSRVCQPPPDVLHAERTQRDKDNES |
| | | PGQEVFYSCEPGYDLRGAASMRCTPQGDWSPAAPTCEVKSCDDEMGQLL |
| | | NGRVLFPVNLQLGAKVDFVCDEGFQLKGSSASYCVLAGMESLWNSSVPV |
| | | CEQIFCPSPPVIPNGRHTGKPLEVFPFGKAVNYTCDPHPDRGTSEDLIG |
| | | ESTIRCTSDPQGNGVWSSPAPRCGILGHCQAPDHELFAKLKTQTNASDE |
| | | PIGTSLKYECRPEYYGRPFSITCLDNLVWSSPKDVCKRKSCKTPPDPVN |
| | | GMVHVITDIQVGSRINYSCTTGHRLIGHSSAECILSGNTAHWSTKPPIC |
| | | QRIPCGLPPTIANGDFISTNRENFHYGSVVTYRCNLGSRGRKVFELVGE |
| | | PSIYCTSNDDQVGIWSGPAPQCII |
| 73 | CCPH₍₂₁₋₂₆₆₎ | LSCPTRNQYVSVKYVNLTNYSGPYPNGTTLHVTCREGYAKRPVQTVTCV |
| | (prt) | NGNWTVPKKCQKKKCSTPQDLLNGRYTVTGNLYYGSVITYTCNSGYSLI |
| | | GSTTSACLLKRGGRVDWTPRPPICDIKKCKPPPQIANGTHTNVKDFYTY |
| | | LDTVTYSCNDETKLTLTGPSSKLCSETGSWVPNGETKCEFIFCKLPQVA |
| | | NAYVEVRKSATSMQYLHINVKCYKGFMLYGETPNTCNHGVWSPAIPECM |
| | | K |
| 74 | ORF4₍₂₂₋₂₆₈₎ | MCHHLPQMPTLTSPKFTPPVKSGTTLQLRCRPGFTPGAGGRKTVTCLGD |
| | (prt) | NMWTTVTPCNRKRCPHVTFPTNGGANYHFKDNDTSPSYGTEAVFYCDPG |
| | | YNLLGESKLFCELQDNNKVGWSGESPICDIKKCAPPEVKSPAYILRAKD |
| | | VYNTREVTRESCPINLKLYGPTHAVCEGPGWTPSTSPFCLEIPCGQPTI |
| | | PHATIQSKPGAPPGSRELVVNCDPGYMPLDGTTLTCEGRGVWKLPLPEC |
| | | VA |
| 75 | EMICE-full | MKVESVTFLTLLGIGCVLSCCTIPSRPINMKFKNNVGTDDTYKVGDTIE |
| | (EVM017, | YVCLPGYRKQKMGSIYVKCTGNGWTLFNQCIKRRCPSPRDIDNGOLDIG |
| | Ectromelia virus) | GTDFGSSITYSCNSGYQLIGESKSYCELGSTGSMVWNPEAPICESVKCQ |
| | (prt) | SPPSISNGRHNGYEDFYTEGSVVTYSCNSGYSLVGNSGVLCSGGEWSDP |
| | | PTCQIVKCPHPTISNGYLSSGFKRSYSYNDNVDFKCKYGYKLSGSSSST |
| | | CSPENTWQPKLPKCVRE |
| 76 | EMICE- | CCTIPSRPINMKFKNNVGTDDTYKVGDTIEYVCLPGYRKQKMGSIYVKC |
| | truncated | TGNGWTLFNQCIKRRCPSPRDIDNGQLDIGGTDFGSSITYSCNSGYQLI |
| | (EVM017, | GESKSYCELGSTGSMVWNPEAPICESVKCQSPPSISNGRHNGYEDFYTE |
| | Ectromelia virus) | GSVVTYSCNSGYSLVGNSGVLCSGGEWSDPPTCQIVKCPHPTISNGYLS |
| | (prt) | SGFKRSYSYNDNVDFKCKYGYKLSGSSSSTCSPENTWQPKLPKCVRE |
| 77 | IMP-full | MKVESVTFLTLLGIGCVLSCCPIPSRPITMKFKGTVDSHYNIGDTIEYL |
| | (CPXV034 protein, | CLPGYRKQKMGPIYAKCTGTGWTLFNQCIKRRCPSPRDIDNGQLDIGGV |
| | Cowpox virus) | DFGSSITYSCNSGYQLIGESKSYCELGSTGSMVWNPEAPICESVKCQSP |
| | (prt) | PSISNGRHNGYEDFYTDGSVVTYSCNSGYSLIGNSAVLCSGGEWSDPPT |
| | | CQIVKCPHPTISNGYLSSGFKRSYSYNDNVDEKCKYGYKLSGSSSSTCS |
| | | PGNTWKPELPKCVR |
| 78 | IMP-truncated | CCPIPSRPITMKFKGTVDSHYNIGDTIEYLCLPGYRKQKMGPIYAKCTG |
| | (CPXV034 protein, | TGWTLFNQCIKRRCPSPRDIDNGOLDIGGVDEGSSITYSCNSGYQLIGE |
| | Cowpox virus) | SKSYCELGSTGSMVWNPEAPICESVKCQSPPSISNGRHNGYEDFYTDGS |
| | (prt) | VVTYSCNSGYSLIGNSAVLCSGGEWSDPPTCQIVKCPHPTISNGYLSSG |
| | | FKRSYSYNDNVDFKCKYGYKLSGSSSSTCSPGNTWKPELPKCVR |

### CLAUSES

Additional expressions of the inventive concept are set out in each of the following numbered clauses.

C1. An isolated nucleic acid encoding a fusion polypeptide, the fusion polypeptide comprising a vaccinia virus envelope protein or part thereof and at least one complement regulatory protein or functional fragment thereof.

C2. The isolated nucleic acid according to Clause C1, wherein the vaccinia virus envelope protein is selected from A13 and A27 or part thereof.

C3. The isolated nucleic acid according to Clause C1 or C2, wherein the vaccinia virus envelope protein is A13.

C4. The isolated nucleic acid according to any preceding clause, wherein:
(i) the vaccinia virus envelope protein is not H3, D8 and/or A27, or part thereof; and/or
(ii) the fusion polypeptide is not a fusion of the vaccinia virus envelope protein H3, D8 or A27 with a complement regulatory protein selected from CD55, CD59, CD46, CD35, factor H and C4-binding protein; and/or
(iii) the fusion polypeptide does not comprise a complement regulatory protein selected from CD55, CD59, CD46, CD35, factor H and C4-binding protein attached to the N-terminus of A27.

C5. The isolated nucleic acid according to any preceding clause, wherein the vaccinia virus envelope protein is an envelope protein from a vaccinia mature virion (MV).

C6. The isolated nucleic acid according to any preceding clause, wherein the at least one complement regulatory protein is selected from one or more of the group consisting of:
(i) CD35, CD55, CD59, CD46, CR1, Factor H, VCP, MOPICE, SPICE, CCPH, C4-binding protein, CD35, Kaposi-sarcoma associated herpesvirus Kaposica/KCP, Herpesvirus saimiri (HVS) and HVS-CD59, Rhesus rhadinovirus RCP-H and RCP-1, murine gammaherpesvirus 68 (yHV-68) RCA, Influenzavirus M1, EMICE and IMP, as well as modified sequences thereof, or functional fragments thereof.

C7. The isolated nucleic acid according to any preceding clause, wherein the at least one complement regulatory protein is selected from one or more of the group consisting of: CD35 CD55, VCP, mutated VCP, SPICE, CCPH and ORF4 or functional fragments thereof.

C8. The isolated nucleic acid according to any preceding clause, wherein the at least one complement regulatory protein or functional fragment thereof is fused to a vaccinia virus MV envelope protein transmembrane region.

C9. The isolated nucleic acid according to Clause 8, wherein the vaccinia virus envelope protein transmembrane region is not H3 or D8.

C10. The isolated nucleic acid according to any preceding clause, wherein the vaccinia virus envelope protein or part thereof is fused to the at least one complement regulatory protein or functional fragment thereof via a peptide linkage.

C11. The isolated nucleic acid according to any preceding clause, wherein the peptide linkage comprises one or more repeats of the sequence (Gly₄Ser); optionally, wherein the peptide linkage comprises (Gly₄Ser), (Gly₄Ser)₂ or (Gly₄Ser)₃.

C12. The isolated nucleic acid according to any preceding clause, wherein: the vaccinia virus envelope protein A13:
(i) is selected from an A13 protein sequence from Vaccinia Copenhagen virus, Camelpox virus, Variola virus, Cowpox virus, Taterapox virus, Monkeypox virus Zaire-96-I-16, Volepox virus, Akhmeta virus, Ectromelia virus, Orthopoxvirus Abatino virus, Skunkpox virus, Raccoonpox virus, Yokapox virus, Murmansk poxvirus, NY_014 poxvirus, and Yaba monkey tumor virus or part thereof; or
(ii) comprises at least amino acids 2 to 21 of a sequence selected from one of SEQ ID NOs: 1 to 16, or a sequence having at least about 90%, at least about 95%, at least about 98% or at least about 99% sequence identity thereto.

C13. The isolated nucleic acid according to any preceding clause, wherein the vaccinia virus envelope protein A13L comprises a sequence selected from:
(i) amino acids 2 to 23 of a sequence selected from one of SEQ ID NOs: 1 to 16;
(ii) amino acids 2 to 25 of a sequence selected from one of SEQ ID NOs: 1 to 16;
(iii) amino acids 2 to 30 of a sequence selected from one of SEQ ID NOs: 1 to 16;
(iv) amino acids 2 to 40 of a sequence selected from one of SEQ ID NOs: 1 to 16;
(v) amino acids 2 to 50 of a sequence selected from one of SEQ ID NOs: 1 to 16;
(vi) amino acids 2 to 60 of a sequence selected from one of SEQ ID NOs: 1 to 16;
(vii) amino acids 2 to 70 of a sequence selected from one of SEQ ID NOs: 1 to 16;
(viii) amino acids 1 to 21 of a sequence selected from one of SEQ ID NOs: 1 to 16; or
(ix) one of SEQ ID NOs: 1 to 16;
   or a sequence having at least about 90%, at least about 95%, at least about 98% or at least about 99% sequence identity thereto.

C14. The isolated nucleic acid according to any preceding clause, wherein:
(i) the at least one complement regulatory protein or functional fragment thereof is joined at or towards the C-terminus of the vaccinia virus envelope protein or part thereof, optionally wherein the vaccinia virus envelope protein is A13; or
(ii) the at least one complement regulatory protein or functional fragment thereof is joined at or towards the N-terminus of the vaccinia virus envelope protein or part thereof, optionally wherein the vaccinia virus envelope protein is A27.

C15. The isolated nucleic acid according to any preceding clause, wherein the at least one complement regulatory protein comprises two, three or four complement regulatory proteins or functional fragments thereof arranged in series; optionally wherein the two, three or four complement regulatory proteins are attached to each other via flexible linker peptides, such as GGGGS or multiple repeats thereof.

C16. The isolated nucleic acid according to Clause C15, wherein the at least one complement regulatory protein comprises two VCP or four VCP proteins or functional fragments thereof arranged in series.

C17. The isolated nucleic acid according to any preceding clause, wherein the fusion polypeptide is selected from: A13 fused to VCP, A13 fused to two VCP proteins arranged in tandem, A13 fused to four VCP proteins arranged in series, A13 fused to a mutant VCP, A13 fused to CD55, A13 fused to CD35, A13 fused to CCPH, or A13 fused to ORF4 or functional fragments thereof;
optionally, wherein the mutant VCP comprises SEQ ID NO: 31; wherein CD55 comprises amino acids 35 to 284 of CD55 (e.g. SEQ ID NO: 71), wherein the CD35 comprises amino acids 42 to 1584 of CD35 (e.g. SEQ ID NO: 72), wherein the CCPH comprises amino acids 21 to 266 of CCPH (e.g. SEQ ID NO: 73), wherein the ORF4 comprises amino acids 22 to 268 of ORF4 (e.g. SEQ ID NO: 74);
optionally wherein the VCP protein is a poxvirus complement control protein or modified poxvirus complement control protein selected from SPICE (SEQ ID NO: 32), MOPICE (SEQ ID NO: 33), EMICE (SEQ ID NOs: 75, 76—particularly SEQ ID NO: 76) or IMP (SEQ ID NOs: 77, 78—particularly SEQ ID NO: 78).

C18. The isolated nucleic acid according to Clause C18, wherein the one or more complement regulatory proteins or functional fragments thereof is fused to the C-terminus of A13.

C19. The isolated nucleic acid according to Clause C17 or Clause C18, which has a sequence selected from any one of SEQ ID NOs: 37 to 46, or a sequence having at least about 80% at least about 90%, at least about 95%, at least about 98% or at least about 99% sequence identity thereto; or which encodes a fusion polypeptide having an amino acid sequence selected from any one of SEQ ID NOs: 47 to 56, or a sequence having at least about 90%, at least about 95%, at least about 98% or at least about 99% sequence identity thereto.

C20. The isolated nucleic acid according to Clause C1 or Clause C2 or any of Clauses C5 to C16 when dependent on Clause C1 or C2, wherein the vaccinia virus envelope protein A27 or part thereof:
(i) is selected from an A27 protein sequence from Vaccinia Copenhagen virus, Vaccinia virus, Cowpox virus, Camelpox virus, Variola virus, Orthopox Abatino virus, Akhmeta virus, Ectromelia virus, Taterapox virus, Monkeypox virus, Volepox virus, Raccoonpox virus, Skunkpox virus or part thereof;
(ii) comprises at least 90 consecutive amino acids of a sequence selected from one of SEQ ID NOs: 17 to 29, or a sequence having at least about 90%, at least about 95%, at least about 98% or at least about 99% sequence identity thereto.

C21. The isolated nucleic acid according to Clause C20, wherein the vaccinia virus envelope protein A27 comprises a sequence selected from one of SEQ ID NOs: 17 to 29, or a sequence having at least about 90%, at least about 95%, at least about 98% or at least about 99% sequence identity thereto.

C22. The isolated nucleic acid according to Clause C20 or Clause C21, wherein the fusion polypeptide is selected from: A27 fused to VCP, A27 fused to a mutant VCP, A27 fused to CD55, A27 fused to CD35, A27 fused to CCPH or A27 fused to ORF4 or functional fragments thereof;
optionally, wherein the mutant VCP comprises SEQ ID NO: 31; wherein CD55 comprises amino acids 35 to 284 of CD55 (e.g. SEQ ID NO: 71), wherein the CD35 comprises amino acids 42 to 1584 of CD35 (e.g. SEQ ID NO: 72), wherein the CCPH comprises amino acids 21 to 266 of CCPH (e.g. SEQ ID NO: 73) or wherein the ORF4 comprises amino acids 22 to 268 of ORF4 (e.g. SEQ ID NO: 74);
optionally wherein the VCP protein is a poxvirus complement control protein or modified poxvirus complement control protein selected from SPICE (SEQ ID NO: 32), MOPICE (SEQ ID NO: 33), EMICE (SEQ ID NOs: 75, 76—particularly SEQ ID NO: 76) or IMP (SEQ ID NOs: 77, 78—particularly SEQ ID NO: 78).

C23. The isolated nucleic acid according to Clause C22, wherein the one or more complement regulatory protein or functional fragment thereof is fused to the N-terminus of A27.

C24. The isolated nucleic acid according to Clause C22 or Clause C23, which has a sequence selected from any one of SEQ ID NOs: 57 to 62 or a sequence having at least about 80%, at least about 90%, at least about 95%, at least about 98% or at least about 99% sequence identity thereto; or which encodes a fusion polypeptide having an amino acid sequence selected from any one of SEQ ID NOs: 63 to 68 or a sequence having at least about 90%, at least about 95%, at least about 98% or at least about 99% sequence identity thereto.

C25. The isolated nucleic acid according to any of Clauses C1 to C24, which comprises an engineered vaccinia virus genome or vector.

C26. An engineered vaccinia virus vector comprising the nucleic acid of any of Clauses C1 to C25.

C27. The engineered vaccinia virus vector according to Clause C26, wherein the nucleic acid encoding a fusion polypeptide comprising a vaccinia virus envelope protein or part thereof and at least one complement regulatory protein or functional fragment thereof is inserted into the vector at a locus outside the corresponding wild-type vaccinia virus envelope protein locus.

C28. The engineered vaccinia virus vector according to Clause C26 or Clause C27, wherein the vaccinia virus vector comprises a deletion of or inactive thymidine kinase (TK) gene.

C29. The engineered vaccinia virus vector according to Clause 28, wherein the nucleic acid encoding the fusion polypeptide is inserted into the locus of the TK gene; optionally wherein the TK gene is deleted or inactivated.

C30. The engineered vaccinia virus vector according to Clauses 26 or Clause 27, wherein the nucleic acid encoding the fusion polypeptide is inserted into the locus of the corresponding envelope protein gene; optionally wherein the corresponding envelope protein gene is deleted or inactivated.

C31. The engineered vaccinia virus vector according to Clause C30, wherein:
(i) the fusion polypeptide comprises A13 or part thereof and the corresponding envelope protein gene locus is the A13L gene locus; or
(ii) the fusion polypeptide comprises A27 or part thereof and the corresponding envelope protein gene locus is the A27L gene locus.

C32. The engineered vaccinia virus vector according to any of Clauses C26 to C31 or the isolated nucleic acid according to any of Clauses C1 to C25, wherein the nucleic acid encoding the fusion polypeptide comprises a vaccinia virus early or late transcription promoter sequence and wherein the early or late transcription promoter sequence is operably linked to the nucleic acid encoding the fusion polypeptide.

C33. The engineered vaccinia virus vector according to any of Clauses C26 to C31 or the isolated nucleic acid according to any of Clauses C1 to C25, wherein the nucleic acid encoding the fusion polypeptide is operably linked to the native promoter for the corresponding vaccinia virus envelope protein.

C34. The engineered vaccinia virus vector according to any of Clauses C26 to C33 or the isolated nucleic acid according to Clause C25, which comprises one or more deletion of or inactive vaccinia virus gene selected from the group: C2L, C1L, N1L, N2L, M1 L, M2L, K1 L, K2L, K3L, K4L, K5L, K6L, K7R, J2R, F1L, F2L, F3L, B14R, B15R, B16R, B17L, B18R, B19R, B20R, K ORF A, K ORF B, B ORF E, B ORF F, B ORF G, B21R, B22R, B23R, B24R, B25R, B26R, B27R, B28R, and B29R.

C35. The engineered vaccinia virus vector according to any of Clauses C26 to C34 or the isolated nucleic acid according to Clause C25, wherein the engineered vaccinia virus vector comprises a nucleic acid sequence encoding:
(i) a cytokine, optionally wherein the cytokine is GM-CSF;
(ii) a pro-drug converting enzyme, optionally wherein the pro-drug converting enzyme is cytosine deaminase (CD);
(iii) a theranostic payload, optionally wherein the theranostic payload is a sodium iodide symporter (NIS);
(iv) an affinity reagent, optionally wherein the affinity reagent is a bispecific antibody;
(v) an imaging or reporter agent, optionally wherein the imaging or reporter agent is luciferase, Renilla luciferase-GFP fusion protein, β-galactosidase, β-glucuronidase, or green fluorescent protein.

C36. The engineered vaccinia virus vector according to any of Clauses C26 to C35 or the isolated nucleic acid according to Clause C25, wherein the engineered vaccinia virus vector comprises a nucleic acid sequence encoding a cytokine; optionally wherein the cytokine is GM-CSF.

C37. The engineered vaccinia virus vector according to any of Clauses C26 to C36 or the isolated nucleic acid according to Clause C25, which comprises an engineered vaccinia virus genome selected from: Abatino macacapox virus, Akhmeta virus, Camelpox virus 903, Camelpox virus CMG, Camelpox virus CMS, Camelpox virus CP1, Camelpox virus CP5, Camelpox virus M-96, Cowpox virus (Brighton Red), Cowpox virus (strain GRI-90), Cowpox virus (strain Hamburg-1985), Cowpox virus (strain Turkmenia-1974), Elephantpox virus, Belo Horizonte virus, Ectromelia virus ERPV, Ectromelia virus Moscow, Ectromelia virus Naval, Ectromelia virus WH, Callithrix jacchus orthopoxvirus, Monkeypox virus (strain Sierra Leone 70-0266), Monkeypox virus (strain Zaire 77-0666), Monkeypox virus Zaire-96-I-16, Raccoonpox virus, Skunkpox virus, Taterapox virus, Aracatuba virus, Buffalopox virus, Cantagalo virus, Guarani P1 virus, Guarani P2 virus, Horsepox virus, Modified Vaccinia Ankara virus, Rabbitpox virus, Rabbitpox virus Utrecht, SPAN 232 virus, Vaccinia Virus Acambis 3000 MVA, Vaccinia virus Ankara, Vaccinia virus Copenhagen, Vaccinia virus Dairen I, Vaccinia virus GLV-1h68, Vaccinia virus IHD-J, Vaccinia virus L-IPV, Vaccinia virus LC16M8, Vaccinia virus LC16MO, Vaccinia virus Lister, Vaccinia virus LIVP, Vaccinia virus Mariana, Vaccinia virus Tashkent, Vaccinia virus Tian Tan, Vaccinia virus WAU86/88-1, Vaccinia virus Western Reserve, Vaccinia virus WR, Vaccinia virus WR 65-16, Vaccinia virus Wyeth, Variola major virus, Variola minor virus, Variola virus human/India/Ind3/1967, Volepox virus, Alaskapox virus, Cetacean poxvirus 1, Dolphin poxvirus 1, Cetacean poxvirus 2, Cowpox-Vaccinia virus, Feline poxvirus ITA1_PG, Feline poxvirus ITA2_BC, Orthopoxvirus GCP2010, Orthopoxvirus GCP2013, Orthopoxvirus NY99014/1999, Orthopoxvirus 0H08/2008, Orthopoxvirus Tena Dona, Orthopoxvirus VPXV_CA85, Orthopoxvirus WA01960/2001, Steller sea lion poxvirus, Orthopoxvirus sp.

C38. The engineered vaccinia virus vector according to any of Clauses C26 to C37 or the isolated nucleic acid according to Clause C25, which comprises an engineered vaccinia virus genome selected from:
(i) Copenhagen, Western Reserve, Wyeth, Lister or Modified Vaccinia Ankara strain;
(ii) Copenhagen or Western Reserve strain; or
(iii) Copenhagen strain.

C39. An engineered vaccinia virus virion derived from the engineered vaccinia virus vector or isolated nucleic acid of any of Clauses C25 to C38.

C40. A modified vaccinia virus virion comprising a nucleic acid according to any of Clauses C1 to C38.

C41. A pharmaceutical composition comprising the engineered vaccinia virus vector according to any of Clauses C26 to C38, the isolated nucleic acid according to any of Clauses C1 to C25 and C32 to C38, the engineered vaccinia virus vector according to any of Clauses C26 to C38, the engineered vaccinia virus virion according to Clause C39, or the modified vaccinia virus virion according to Clause C40 and a pharmaceutically acceptable carrier.

C42. The pharmaceutical composition according to Clause C41, which is formulated for systemic or local or regional administration.

C43. The pharmaceutical composition according to Clause C41 or Clause C42, which comprises a modified or engineered vaccinia virus virion and is formulated to have a unit dose of:
(i) between about 1×10³ and about 1×10¹⁵ pfu per ml;
(ii) between about 1×10⁴ and about 1×10¹⁴ pfu per ml; or
(iii) between about 1×10⁶ and about 1×10¹² pfu per ml.

C44. An engineered vaccinia virus vector according to any of Clauses C26 to C38, an isolated nucleic acid according to any of Clauses C1 to C25 and C32 to C38, an engineered or modified vaccinia virus virion according to Clause C39 or Clause C40, or a pharmaceutical composition according to any of Clauses C41 to C43 for use in a method of treating cancer and/or proliferative diseases or disorders in a subject.

C45. A method for treating cancer and/or proliferative diseases or disorders in a mammalian subject, the method comprising administering to the subject a therapeutically effective amount of the engineered vaccinia virus vector according to any of Clauses C26 to C38, the isolated nucleic acid according to any of Clauses C1 to C25 and C32 to C38, the engineered or modified vaccinia virus virion according to Clause C39 or Clause C40, or the pharmaceutical composition of any of Clauses C41 to C43.

C46. The engineered vaccinia virus vector, isolated nucleic acid, engineered or modified vaccinia virus virion or pharmaceutical composition for use according to Clause C44, or the method according to Clause C45, wherein the cancer and/or proliferative diseases or disorders is selected from: lung cancers (e.g. lung adenocarcinomas), cervical cancer, breast cancer, cardiac cancer, colon cancer, prostrate cancer, brain glioblastoma, pancreatic cancer, leukemia (e.g. acute monocytic leukemia), lymphoma, kidney cancer, colorectal cancer, bladder cancer, testicular cancer, gastrointestinal cancer, liver cancer (e.g. hepatocarcinoma), and/or glioblastoma. The invention may also be useful in the treatment of one or more of skin cancer (e.g. melanoma), head and/or neck cancer, gallbladder cancer, uterine cancer, stomach cancer, tyroid cancer, laryngeal cancer, lip and/or oral cancer, throat cancer, ocular cancer and bone cancer.

C47. The engineered vaccinia virus vector, isolated nucleic acid, engineered or modified vaccinia virus virion or pharmaceutical composition for use according to Clause C44, or the method according to Clause C45, wherein the cancer and/or proliferative diseases or disorders is selected from any one or more of the group consisting of hepatocarcinoma (HC), acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, wilms tumor and other childhood kidney tumors, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi sarcoma, rhabdomyosarcoma, Sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

C48. The engineered vaccinia virus vector, isolated nucleic acid, engineered or modified vaccinia virus virion or pharmaceutical composition for use according to any of Clauses C44, C46 or C47, or the method according to any of Clauses C45 to C47, wherein the subject is selected from a human, a non-human primate, a cow, a sheep, a pig, a dog, a cat, a rabbit, a bat, a mouse or a rat; optionally wherein the subject is a human, a non-human primate, a rabbit, or a mouse; optionally wherein the subject is a human.

C49. The engineered vaccinia virus vector, isolated nucleic acid, engineered or modified vaccinia virus virion or pharmaceutical composition for use according to any Clauses C44 or C46 to C48, or the method according to any of Clauses C45 to C48, wherein the vaccinia virus vector, isolated nucleic acid, engineered or modified vaccinia virus virion or pharmaceutical composition is administered to the subject systemically or locally; optionally, wherein administration is by a route selected from intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage or oral.

C50. The engineered vaccinia virus vector, isolated nucleic acid, engineered or modified vaccinia virus virion or pharmaceutical composition for use according to any of Clauses C44 or C46 to C49, or the method according to any of Clauses C45 to C49, wherein the vaccinia virus vector, isolated nucleic acid, engineered or modified vaccinia virus virion or pharmaceutical composition is administered in combination with one or more additional therapeutic agent or therapy, and wherein administration of the one or more additional therapeutic agent or therapy is simultaneous, separate or sequential to the administration of the vaccinia virus vector, isolated nucleic acid, engineered or modified vaccinia virus virion or pharmaceutical composition.

C51. The engineered vaccinia virus vector, isolated nucleic acid, engineered or modified vaccinia virus virion or pharmaceutical composition for use according to Clause C50, or the method according to Clause C50, wherein the one or more additional therapeutic agent or therapy comprises chemotherapy, radiation therapy, oncolytic viral therapy, an immunomodulatory protein, an anti-cancer agent, or any combination thereof.

C52. The engineered vaccinia virus vector, isolated nucleic acid, engineered or modified vaccinia virus virion, or pharmaceutical composition for use, or the method according to Clause C51, wherein the anti-cancer agent is selected from one or more chemotherapeutic agent, radiotherapeutic agent, cytokine, immune checkpoint inhibitor, anti-angiogenic agent, apoptosis-inducing agent, anti-cancer antibody and/or anti-cyclin-dependent kinase agent.

C53. The engineered vaccinia virus vector, isolated nucleic acid, engineered or modified vaccinia virus virion, or pharmaceutical composition for use, or the method according to Clause C51 or Clause C52, wherein the therapy is selected from one or more of chemotherapy, biological therapy, radiotherapy, immunotherapy, hormone therapy, anti-vascular therapy, cryotherapy, toxin therapy and/or surgery or combinations thereof.

C54. The engineered vaccinia virus vector, isolated nucleic acid, engineered or modified vaccinia virus virion or pharmaceutical composition for use according to any of Clauses C44 or C46 to C53, or the method according to any of Clauses C45 to C53, wherein the cancer is a primary cancer, a secondary cancer or a metastasis.

C55. The engineered vaccinia virus vector, isolated nucleic acid, engineered or modified vaccinia virus virion or pharmaceutical composition for use according to any of Clauses C44 or C46 to C54, or the method according to any of Clauses C45 to C54, wherein the method reduces the size of a tumour or the rate or proliferation of tumour cells in the subject by between about 10% and about 100%, by between about 20% and about 90%, by between about 30% and about 80%, or between about 40% and about 70%.

C56. The engineered or modified vaccinia virus virion for use according to any of Clauses C44 or C46 to C55, or method comprising an engineered or modified vaccinia virus virion according to any of Clauses C45 to C55, wherein the engineered or modified vaccinia virus virion is administered to the subject at a dose of:
(i) between about 1×10³ and about 1×10¹⁵ pfu per kg;
(ii) between about 1×10⁴ and about 1×10¹⁴ pfu per kg; or
(iii) between about 1×10⁶ and about 1×10¹² pfu per kg.

C57. A kit comprising one or more first active agent selected from: an engineered vaccinia virus vector according to any of Clauses C26 to C38, an isolated nucleic acid according to any of Clauses C1 to C25 and C32 to C38, an engineered or modified vaccinia virus virion according to Clause C39 or Clause C40, or a pharmaceutical composition according to any of Clauses C41 to C43, and a container housing the first active agent; and optionally, one or more of:
one or more second active agent selected from the group consisting of an anti-cancer agent, an immunomodulatory agent, or any combinations thereof, that may be administered to a subject in combination with the one or more first active agent;
one or more device or apparatus for administering the at least one first and optional second active agents; and
instructions for use of the kit.

C58. An isolated polypeptide encoded by the nucleic acid of any of Clauses C1 to C25.

C59. The isolated polypeptide according to Clause A58, which has a selected from any one of SEQ NOs: 47 to 56 or 63 to 68, or a sequence having at least about 90%, at least about 95%, at least about 98% or at least about 99% sequence identity thereto.

1. An isolated nucleic acid encoding a fusion polypeptide, the fusion polypeptide comprising a vaccinia virus envelope protein or part thereof and at least one complement regulatory protein or functional fragment thereof.

2. The isolated nucleic acid according to claim 1, wherein the vaccinia virus envelope protein is selected from A13 and A27 or part thereof.

3. The isolated nucleic acid according to claim 1 or claim 2, wherein the vaccinia virus envelope protein is A13.

4. The isolated nucleic acid according to any preceding claim, wherein the vaccinia virus envelope protein is an envelope protein from a vaccinia mature virion (MV).

5. The isolated nucleic acid according to any preceding claim, wherein the at least one complement regulatory protein is selected from one or more of the group consisting of:
(i) CD35, CD55, CD59, CD46, CR1, Factor H, VCP, MOPICE, SPICE, CCPH, C4-binding protein, CD35, Kaposi-sarcoma associated herpesvirus Kaposica/KCP, Herpesvirus saimiri (HVS) and HVS-CD59, Rhesus rhadinovirus RCP-H and RCP-1, murine gammaherpesvirus 68 (yHV-68) RCA, Influenzavirus M1, EMICE and IMP, as well as modified sequences thereof, or functional fragments thereof.

6. The isolated nucleic acid according to any preceding claim, wherein the at least one complement regulatory protein is selected from one or more of the group consisting of: CD35 CD55, VCP, mutated VCP, SPICE, CCPH and ORF4 or functional fragments thereof.

7. The isolated nucleic acid according to any preceding claim, wherein the at least one complement regulatory protein or functional fragment thereof is fused to a vaccinia virus MV envelope protein transmembrane region.

8. The isolated nucleic acid according to claim 7, wherein the vaccinia virus envelope protein transmembrane region is not H3 or 08.

9. The isolated nucleic acid according to any preceding claim, wherein: the vaccinia virus envelope protein A13:
(i) is selected from an A13 protein sequence from Vaccinia Copenhagen virus, Camelpox virus, Variola virus, Cowpox virus, Taterapox virus, Monkeypox virus Zaire-96-I-16, Volepox virus, Akhmeta virus, Ectromelia virus, Orthopoxvirus Abatino virus, Skunkpox virus, Raccoonpox virus, Yokapox virus, Murmansk poxvirus, NY_014 poxvirus, and Yaba monkey tumor virus or part thereof; or
(ii) comprises at least amino acids 2 to 21 of a sequence selected from one of SEQ ID NOs: 1 to 16, or a sequence having at least about 90%, at least about 95%, at least about 98% or at least about 99% sequence identity thereto.

10. The isolated nucleic acid according to any preceding claim, wherein the fusion polypeptide is selected from: A13 fused to VCP, A13 fused to two VCP proteins arranged in tandem, A13 fused to four VCP proteins arranged in series, A13 fused to a mutant VCP, A13 fused to CD55, A13 fused to CD35, A13 fused to CCPH, or A13 fused to ORF4 or functional fragments thereof;
optionally, wherein the mutant VCP comprises SEQ ID NO: 31; wherein CD55 comprises amino acids 35 to 284 of CD55 (e.g. SEQ ID NO: 71), wherein the CD35 comprises amino acids 42 to 1584 of CD35 (e.g. SEQ ID NO: 72), wherein the CCPH comprises amino acids 21 to 266 of CCPH (e.g. SEQ ID NO: 73) or wherein the ORF4 comprises amino acids 22 to 268 of ORF4 (e.g. SEQ ID NO: 74);
optionally wherein the VCP protein is a poxvirus complement control protein or modified poxvirus complement control protein selected from SPICE (SEQ ID NO: 32), MOPICE (SEQ ID NO: 33), EMICE (SEQ ID NOs: 75, 76—particularly SEQ ID NO: 76) or IMP (SEQ ID NOs: 77, 78—particularly SEQ ID NO: 78).

11. The isolated nucleic acid according to any preceding claim, wherein the one or more complement regulatory proteins or functional fragments thereof is fused to the C-terminus of A13.

12. The isolated nucleic acid according to claim 10 or claim 11, which has a sequence selected from any one of SEQ ID NOs: 37 to 46, or a sequence having at least about 80% at least about 90%, at least about 95%, at least about 98% or at least about 99% sequence identity thereto; or which encodes a fusion polypeptide having an amino acid sequence selected from any one of SEQ ID NOs: 47 to 56, or a sequence having at least about 90%, at least about 95%, at least about 98% or at least about 99% sequence identity thereto.

13. The isolated nucleic acid according to claim 1 or claim 2 or any of claims 4 to 8 when dependent on claim 1 or claim 2, wherein the fusion polypeptide is selected from: A27 fused to VCP, A27 fused to a mutant VCP, A27 fused to CD55, A27 fused to CD35, A27 fused to CCPH or A27 fused to ORF4 or functional fragments thereof;
optionally, wherein the mutant VCP comprises SEQ ID NO: 31; wherein CD55 comprises amino acids 35 to 284 of CD55 (e.g. SEQ ID NO: 71), wherein the CD35 comprises amino acids 42 to 1584 of CD35 (e.g. SEQ ID NO: 72), wherein the CCPH comprises amino acids 21 to 266 of CCPH (e.g. SEQ ID NO: 73) or wherein the ORF4 comprises amino acids 22 to 268 of ORF4 (e.g. SEQ ID NO: 74);
optionally wherein the VCP protein is a poxvirus complement control protein or modified poxvirus complement control protein selected from SPICE (SEQ ID NO: 32), MOPICE (SEQ ID NO: 33), EMICE (SEQ ID NOs: 75, 76—particularly SEQ ID NO: 76) or IMP (SEQ ID NOs: 77, 78—particularly SEQ ID NO: 78).

14. The isolated nucleic acid according to claim 13, wherein the one or more complement regulatory protein or functional fragment thereof is fused to the N-terminus of A27.

15. The isolated nucleic acid according to claim 13 or claim 14, which has a sequence selected from any one of SEQ ID NOs: 57 to 62 or a sequence having at least about 80%, at least about 90%, at least about 95%, at least about 98% or at least about 99% sequence identity thereto; or which encodes a fusion polypeptide having an amino acid sequence selected from any one of SEQ IN NOs: 63 to 68 or a sequence having at least about 90%, at least about 95%, at least about 98% or at least about 99% sequence identity thereto.

16. An engineered vaccinia virus vector comprising the nucleic acid of any of claims 1 to 15.

17. The engineered vaccinia virus vector according to claim 16, wherein the nucleic acid encoding a fusion polypeptide comprising a vaccinia virus envelope protein or part thereof and at least one complement regulatory protein or functional fragment thereof is inserted into the vector at a locus outside the corresponding wild-type vaccinia virus envelope protein locus.

18. The engineered vaccinia virus vector according to claim 16 or claim 17, wherein the vaccinia virus vector comprises a deletion of or inactive thymidine kinase (TK) gene.

19. The engineered vaccinia virus vector according to claim 18, wherein the nucleic acid encoding the fusion polypeptide is inserted into the locus of the TK gene; optionally wherein the TK gene is deleted or inactivated.

20. The engineered vaccinia virus vector according to any of claims 16 to 19 or the isolated nucleic acid according to any of claims 1 to 15, wherein the nucleic acid encoding the fusion polypeptide is operably linked to the native promoter for the corresponding vaccinia virus envelope protein.

21. The engineered vaccinia virus vector according to any of claims 16 to 20 or the isolated nucleic acid according to any of claims 1 to 15, which comprises an engineered vaccinia virus genome selected from:
(i) Copenhagen, Western Reserve, Wyeth, Lister or Modified Vaccinia Ankara strain;
(ii) Copenhagen or Western Reserve strain; or
(iii) Copenhagen strain.

22. A modified vaccinia virus virion comprising a nucleic acid according to any of claims 1 to 21.

23. A pharmaceutical composition comprising the engineered vaccinia virus vector according to any of claims 16 to 21, the isolated nucleic acid according to any of claims 1 to 15, claim 20 or claim 21, or the modified vaccinia virus virion according to claim 22 and a pharmaceutically acceptable carrier.

24. The pharmaceutical composition according to claim 23, which is formulated for systemic or local or regional administration.

25. The pharmaceutical composition according to claim 23 or claim 24, which comprises a modified vaccinia virus virion and is formulated to have a unit dose of:
(i) between about 1×10³ and about 1×10¹⁵ pfu per ml;
(ii) between about 1×10⁴ and about 1×10⁴ pfu per ml; or
(iii) between about 1×10⁶ and about 1×10¹² pfu per ml.

26. The engineered vaccinia virus vector according to any of claims 16 to 21, an isolated nucleic acid according to any of claims 1 to 15, claim 20 or claim 21, the modified vaccinia virus virion according to claim 22, or a pharmaceutical composition according to any of claims 23 to 25 for use in a method of treating cancer and/or proliferative diseases or disorders in a subject.

27. A method for treating cancer and/or proliferative diseases or disorders in a mammalian subject, the method comprising administering to the subject a therapeutically effective amount of the engineered vaccinia virus vector according to any of claims 16 to 21, an isolated nucleic acid according to any of claims 1 to 15, claim 20 or claim 21, the modified vaccinia virus virion according to claim 22, or a pharmaceutical composition according to any of claims 23 to 25.

28. The engineered vaccinia virus vector, isolated nucleic acid, modified vaccinia virus virion or pharmaceutical composition for use according to claim 26, or the method according to claim 27, wherein the cancer and/or proliferative diseases or disorders is selected from: lung cancers (e.g. lung adenocarcinomas), cervical cancer, breast cancer, cardiac cancer, colon cancer, prostrate cancer, brain glioblastoma, pancreatic cancer, leukemia (e.g. acute monocytic leukemia), lymphoma, kidney cancer, colorectal cancer, bladder cancer, testicular cancer, gastrointestinal cancer, liver cancer (e.g. hepatocarcinoma), and/or glioblastoma. The invention may also be useful in the treatment of one or more of skin cancer (e.g. melanoma), head and/or neck cancer, gallbladder cancer, uterine cancer, stomach cancer, tyroid cancer, laryngeal cancer, lip and/or oral cancer, throat cancer, ocular cancer and bone cancer.

29. The engineered vaccinia virus vector, isolated nucleic acid, modified vaccinia virus virion or pharmaceutical composition for use according to claim 26 or claim 28, or the method according to claim 27 or claim 28, wherein the vaccinia virus vector, isolated nucleic acid, engineered or modified vaccinia virus virion or pharmaceutical composition is administered to the subject systemically or locally; optionally, wherein administration is by a route selected from intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage or oral.

30. The engineered vaccinia virus vector, isolated nucleic acid, modified vaccinia virus virion or pharmaceutical composition for use according to any of claims 26, 28 or 29, or the method according to any of claims 27 to 29, wherein the vaccinia virus vector, isolated nucleic acid, engineered or modified vaccinia virus virion or pharmaceutical composition is administered in combination with one or more additional therapeutic agent or therapy, and wherein administration of the one or more additional therapeutic agent or therapy is simultaneous, separate or sequential to the administration of the vaccinia virus vector, isolated nucleic acid, engineered or modified vaccinia virus virion or pharmaceutical composition.

31. The engineered vaccinia virus vector, isolated nucleic acid, modified vaccinia virus virion or pharmaceutical composition for use according to any of claim 26 or 28 to 30, or the method according to any of claims 27 to 30, wherein the cancer is a primary cancer, a secondary cancer or a metastasis.

32. The modified vaccinia virus virion for use according to any of claim 26 or 28 to 31, or method comprising a modified vaccinia virus virion according to any of claims 27 to 31, wherein the modified vaccinia virus virion is administered to the subject at a dose of:
(i) between about 1×10³ and about 1×10¹⁵ pfu per kg;
(ii) between about 1×10⁴ and about 1×10⁴ pfu per kg; or
(iii) between about 1×10⁶ and about 1×10¹² pfu per kg.


SEQUENCE LISTING (78 sequences)

SEQ ID NO: 1 - protein, 70 aa, Vaccinia virus
MIGILLLIGICVAVTVAILYSMYNKIKNSQNPNPSPNLNSPPPEPKNTKFVNNLEKDHISSLYNLVKSSV

SEQ ID NO: 2 - protein, 70 aa, Camelpox virus
MIGILLLIGICVAVTVAILYAMYNKIKNSQNPNPSPNVNSPPSETRNTKFVNNLEKDHISSLYNLVKSSA

SEQ ID NO: 3 - protein, 68 aa, Variola virus
MIGILLLIGICVAVTVAILYAMYNKIKNSQNPSPNVNLPPPETRNTRFVNNLEKDHISSLYNLVKSSV

SEQ ID NO: 4 - protein, 69 aa, Cowpox virus
MIGILLLIGICVAVTVAILAMYNKIKNSQNPNPSPNLNSPPPETRNTKFVNNLEKDHISSLYNLVKSSA

SEQ ID NO: 5 - protein, 68 aa, Taterapox virus
MIGILLLIGICVAVTVAILYAMYNKIKNSQNPSPNVNSPPPETRNTKFVNNLEKDHISSLYNLVKSSA

SEQ ID NO: 6 - protein, 70 aa, Monkeypox virus
MIGILLLIGICVAVTVAILYTLYNKIKNPQNPNPSPNLNSPPPETRNTKFVNNLEKDHISSLYNLVKSSA

SEQ ID NO: 7 - protein, 66 aa, Volepox virus
MIGTIILIVICVTITAAIIYALYNRTKNQQKQNTNTPSPEPRNTKFVNNLNKDHITSLYNLVKSSS

SEQ ID NO: 8 - protein, 72 aa, Akhmeta virus
MIGILLLIGICVAVTVAILYALYNKIKNPQNPNPNPNPNLNSPPPETRNTKFVNNLEKEHISSLYNLVKSSA

SEQ ID NO: 9 - protein, 66 aa, Ectromelia virus
MIGILLLIGICVAVTVAILYALYNKIKSPQNPDPNPPPPEPRNTKFVNNLEKDHISSLYNLVKSSA

SEQ ID NO: 10 - protein, 66 aa, Orthopoxvirus Abatino virus
MIGILLLIGICVAVTVAILYALYNKIKNPQNPDPNPPPPEPRNTKFVNNLEKDHISSLYNLVKSSA

SEQ ID NO: 11 - protein, 66 aa, Skunkpox virus
MIGTIILIVICVTITAAIIYALYNRTKSQQKPNPPAPTPEPRNTKFVNNLEKDHITSLYNLVKSSS

SEQ ID NO: 12 - protein, 66 aa, Raccoon virus
MIGTIILIIICVTITAAIIYALYNRTKNQQNQNPPAPSPEPRNTKFVNNLEKDHITSLYNLVKSSS

SEQ ID NO: 13 - protein, 61 aa, Yokapox virus
MILSIILIGVCIVIVGGIIYAVYYRNKQRPNNSNNNKKTKYVNSLGKEHIDSLYNLFKSSP

SEQ ID NO: 14 - protein, 62 aa, Murmansk virus
MIGNIILIVLCVVVVGGIIYALYHRTQKQPNPPPNNDRKTKYVNSLGKEHISSLYNLFKSSS

SEQ ID NO: 15 - protein, 62 aa, NY_014 poxvirus
MIGNIILIVLCVVVVGGIIYALYHRTQKQPNPPPNNDMKTKYVNSLGKEHISSLYNLFKSSS

SEQ ID NO: 16 - protein, 68 aa, Yaba monkey tumor virus
MIADIILVIICVAIVGLIVYGVYNKKSANLFNEKQEEYKIEDIKTSYVDRLKPNHLNSFYKLFSGQFN

SEQ ID NO: 17 - protein, 110 aa, Vaccinia Copenhagen virus
MDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 18 - protein, 110 aa, Vaccinia virus
MDGTLFPGDDDLAIPATEFFSTKAAKKPEAKREAIVKADEDDNEETLKQRLTNLEKKITNVTTKFEQIEKCCKRNDEVLFRLENHAETLRAAMISLAKKIDVQTGRRPYE

SEQ ID NO: 19 - protein, 110 aa, Cowpox virus
MDGTLFPGDDDLAIPATEFFSTKAAKKPEAKREAIVKAEGDDNEETLKQRLTNLEKKITNVTTKFEQIEKCCKRNDEVLFRLENHAETLRAAMISLAKKIDVQTGRRPYE

SEQ ID NO: 20 - protein, 117 aa, Camelpox virus
MDGTLFPGDDDLAIPATEFFSTKAAKKPEAKREAIIKADGDDNEETLKQRLTNLEKKITNVTTKFEQIEKCCKRNDEVLFRLENHAETLRAAMISLAKKIDVQTGRRPYDNLTLLFN

SEQ ID NO: 21 - protein, 110 aa, Variola virus
MDGTLFPGDDDLAIPATEFFSTKAAKKPEAKREAIVKADGDNNEETLKQRLTNLEKKITNVTTKFEQIEKCCKRNDDVLFRLENHAETLRAAMISLAKKIDVQTGRRPYE

SEQ ID NO: 22 - protein, 110 aa, Orthopoxvirus Abatino virus
MDGTLFPGDDDLAIPATEFFSTKAAKKPEAKREAIVKADGDDNEETLKQRLTNLEKKINNVTTKFEQIEKCCKRNDEVLFRLENHAETLRAAMISLAKKIDVQTGRRTYE

SEQ ID NO: 23 - protein, 110 aa, Akhmeta virus
MDGTLFPGDDDLAIPATEFFSTKAAKKIEAKREVIVKADGDDSEETLKQRLTNLEKKITNVTTKFEQIEKCCKRNDEVLFRLENHAETLRAAMISLAKKIDVQTGRRPYE

SEQ ID NO: 24 - protein, 110 aa, Ectromelia virus
MDGTLFPGDDDLAIPATEFFSTKAAKKPEDKHEATVKADGDDNEETLKQRLTNLEKKITNVTTKFEQIEKCCKRNDEVLFRLENHAETLRAAMISLAKKIDVQTGRRPYE

SEQ ID NO: 25 - protein, 110 aa, Taterapox virus
MDGTLFPGDDDLAIPATEFFSTKAAKKPEAKREAIVKADGYDNEETLKQRLTNLEKKITNVTIKFEQIEKCCKRNDEVLFRLENHAETLRAAMISLAKKIDVQTGRHPYE

SEQ ID NO: 26 - protein, 110 aa, Monkeypox virus
MDGTLFPGDDDLAIPATEFFSTKAAKNPETKREAIVKAYGDDNEETLKQRLTNLEKKITNITTKFEQIEKCCKHNDEVLFRLENHAETLRAAMISLAKKIDVQTGRRPYE

SEQ ID NO: 27 - protein, 132 aa, Volepox virus
MDGTLFPGDDDIAIPATEFFVNRSAKKPEESVKSKVSKQKRKAVVKADGEDDPDEDDDDDEEEEDDETIKDRLTNLEKKITNVTTKFAQIEKCCKRNDEVLFRLENHAETLRAAMLALAKKIDVQTGRQRYE

SEQ ID NO: 28 - protein, 119 aa, Raccoon virus
MEGTLFPGDDDIAIPATEFFVNKAAKKPEKPAKRKVVKADDAEEKADEEEDAEEDIKGRLTNLEKKITNVTTKFAQIEKCCKRNDEVLFRLENHAETLRVAMLSLAKKIDIQTGRQRYE

SEQ ID NO: 29 - protein, 136 aa, Skunkpox virus
MDGTLFPGDDDMAIPATEFFVTRAAKKPEEPVKRKVVKNKNKHKVVKADGEDDPDEDDEDDDDEEDDDAEETIKHRLTNLEKKITNVTTKFAQIEKCCKRNDEVLFRLENHAETLRAAMLTLAKKIDVQTGRQRYE

SEQ ID NO: 30 - protein, 244 aa, Vaccinia virus
SCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 31 - protein, 244 aa, Vaccinia virus
SCTIPSRPINMKFKNSVETDANANYNIGDTIEYLCLPGYRKQKMGPIYAKCTGTGWTLFNQCIKRRCPSPRDIDNGHLDIGGVDFGSSITYSCNSGYHLIGESKSYCELGSTGSMVWNPKAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 32 - protein, 246 aa, synthetic construct
LSCCTIPSRPINMTFKNSVETDANANYNIGDTIEYLCLPGYRKQKMGPIYAKCTGTGWTLFNQCIKRRCPSPRDIDNGHLDIGGVDFGSSITYSCNSGYYLIGEYKSYCKLGSTGSMVWNPKAPICESVKCQLPPSISNGRHNGYNDFYTDGSVVTYSCNSGYSLIGNSGVLCSGGEWSNPPTCQIVKCPHPTILNGYLSSGFKRSYSYNDNVDFTCKYGYKLSGSSSSTCSPGNTWQPELPKCVR

SEQ ID NO: 33 - protein, 199 aa, synthetic construct
LSYCTIPSRPINMKFKNSVETDANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYIDGSIVTYSCNSGYSLIGNSGVMCSGGEWSNPPTCQIVKCPHPISNGKLLAA

SEQ ID NO: 34 - protein, 5 aa, synthetic construct
GGGGS

SEQ ID NO: 35 - protein, 10 aa, synthetic construct
GGGGSGGGGS

SEQ ID NO: 36 - protein, 15 aa, synthetic construct
GGGGSGGGGSGGGGS

SEQ ID NO: 37 - DNA, 957 bp, synthetic construct
  source 1..957: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggaggaggtggatcctcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgccacgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgc

SEQ ID NO: 38 - DNA, 1704 bp, synthetic construct
  source 1..1704: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggcggaggtggaagttcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgcggcggaggtggaagttcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgc

SEQ ID NO: 39 - DNA, 3198 bp, synthetic construct
  source 1..3198: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggcggaggtggaagttcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgcggcggaggtggaagttcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgcggcggaggtggaagttcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgcggcggaggtggaagttcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgcctcgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgc

SEQ ID NO: 40 - DNA, 957 bp, synthetic construct
  source 1..957: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggaggaggtggatcctcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgccacgagatatcgataatggccaccttgatattggtggagtagactttggctctagtataacgtactcttgtaatagcggatatcatttgatcggtgaatctaaatcgtattgtgaattaggatctactggatctatggtatggaatcccaaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgc

SEQ ID NO: 41 - DNA, 975 bp, synthetic construct
  source 1..975: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggaggaggtggatccgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcaga

SEQ ID NO: 42 - DNA, 4884 bp, synthetic construct
  source 1..4884: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggcggaggtggaagtggaggcggtggatccggtggaggaggcagccaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattata

SEQ ID NO: 43 - DNA, 963 bp, synthetic construct
  source 1..963: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggaggaggtggatccttaagctgtcctacacgtaaccagtatgtttctgtcaaatatgtgaatctaactaactattcaggcccgtatccaaacgggacaacgctacacgtgacatgccgtgaaggatatgcaaaaagaccagtacaaactgttacatgcgtcaatggtaactggactgtacctaaaaagtgtcagaaaaagaaatgttctacaccgcaagatcttttaaatggaagatatactgtaactggtaatttatattacggttcagttatcacttatacttgtaattcaggctacagcttaattggaagcacaacatcagcttgtttacttaaacgaggtggtcgtgttgactggactccacgacctccaatttgtgacattaaaaaatgtaaacctcctccacaaatagctaatgggactcacactaatgtcaaagatttctatacttatttagatacagttacgtactcatgcaatgacgaaacaaagttaactttaacaggcccttcatcgaaactttgttcagaaactggctcatgggtacctaatggagaaactaagtgtgaatttatattttgtaaactacctcaagttgcgaatgcgtacgttgaagttagaaagtcagctacgagcatgcaatatttgcatataaatgttaaatgttataaaggatttatgctatatggagaaactcctaatacgtgtaaccatggagtatggtctccagctattcctgaatgtatgaag

SEQ ID NO: 44 - DNA, 966 bp, synthetic construct
  source 1..966: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggcggaggtggaagtatgtgccaccatcttcctcaaatgcccacactaacatctcctaaatttacaccaccagtcaaaagtggcaccaccctgcagctccgctgtaggccagggttcacaccaggcgcaggggggcggaagacagtaacctgcttaggggataatatgtggacaaccgtgactccctgtaacagaaagaggtgcccacacgttacctttccaaccaatgggggcgctaattaccattttaaagacaatgataccagtcctagctatggtaccgaggcagtgttttactgtgatcccggctacaatcttctgggagaaagtaaattattttgtgagctgcaggataataataaagtgggctggagtggggaatctcccatatgtgacataaaaaagtgtgcacctcccgaagtcaagtccccagcctatatactgagggccaaagatgtgtataacacccgagaggtgaccagatttagttgtcctataaatctgaagctatatgggccaacccacgcagtctgtgagggacccgggtggacaccatccaccagtcccttttgcctggaaattccctgtggccaacctacaattccgcatgccacaattcaatctaagccaggggctcccccaggctctagagaacttgttgtcaattgcgatccaggttacatgcccctagatgggaccaccttgacatgcgaaggcagaggtgtttggaaactgccccttcctgaatgtgttgcc

SEQ ID NO: 45 - DNA, 960 bp, synthetic construct
  source 1..960: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtagactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcaga

SEQ ID NO: 46 - DNA, 1023 bp, synthetic construct
  source 1..1023: mol_type = other DNA
atgattggtattcttttgttgatcggtatttgcgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtaggaggaggtggatccgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaccggtaagcctatccctaaccctctcctcggtctcgattctacg

SEQ ID NO: 47 - protein, 319 aa, synthetic construct
MIGILLLIGICVAVTVAILYSMYNKIKNSQNPNPSPNLNSPPPEPKNTKFVNNLEKDHISSLYNLVKSSVGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 48 - protein, 568 aa, synthetic construct
MIGILLLIGICVAVTVAILYSMYNKIKNSQNPNPSPNLNSPPPEPKNTKFVNNLEKDHISSLYNLVKSSVGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVRGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 49 - protein, 1066 aa, synthetic construct
MIGILLLIGICVAVTVAILYSMYNKIKNSQNPNPSPNLNSPPPEPKNTKFVNNLEKDHISSLYNLVKSSVGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVRGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVRGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVRGGGGSSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 50 - protein, 319 aa, synthetic construct; identical to SEQ ID NO: 47

SEQ ID NO: 51 - protein, 325 aa, synthetic construct
MIGILLLIGICVAVTVAILYSMYNKIKNSQNPNPSPNLNSPPPEPKNTKFVNNLEKDHISSLYNLVKSSVGGGGSDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECR

SEQ ID NO: 52 - protein, 1628 aa, synthetic construct
MIGILLLIGICVAVTVAILYSMYNKIKNSQNPNPSPNLNSPPPEPKNTKFVNNLEKDHISSLYNLVKSSVGGGGSGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 53 - protein, 321 aa, synthetic construct
MIGILLLIGICVAVTVAILYSMYNKIKNSQNPNPSPNLNSPPPEPKNTKFVNNLEKDHISSLYNLVKSSVGGGGSLSCPTRNQYVSVKYVNLTNYSGPYPNGTTLHVTCREGYAKRPVQTVTCVNGNWTVPKKCQKKKCSTPQDLLNGRYTVTGNLYYGSVITYTCNSGYSLIGSTTSACLLKRGGRVDWTPRPPICDIKKCKPPPQIANGTHTNVKDFYTYLDTVTYSCNDETKLTLTGPSSKLCSETGSWVPNGETKCEFIFCKLPQVANAYVEVRKSATSMQYLHINVKCYKGFMLYGETPNTCNHGVWSPAIPECMK

SEQ ID NO: 54 - protein, 322 aa, synthetic construct
MIGILLLIGICVAVTVAILYSMYNKIKNSQNPNPSPNLNSPPPEPKNTKFVNNLEKDHISSLYNLVKSSVGGGGSMCHHLPQMPTLTSPKFTPPVKSGTTLQLRCRPGFTPGAGGRKTVTCLGDNMWTTVTPCNRKRCPHVTFPTNGGANYHFKDNDTSPSYGTEAVFYCDPGYNLLGESKLFCELQDNNKVGWSGESPICDIKKCAPPEVKSPAYILRAKDVYNTREVTRFSCPINLKLYGPTHAVCEGPGWTPSTSPFCLEIPCGQPTIPHATIQSKPGAPPGSRELVVNCDPGYMPLDGTTLTCEGRGVWKLPLPECVA

SEQ ID NO: 55 - protein, 320 aa, synthetic construct
MIGILLLIGICVAVTVAILYSMYNKIKNSQNPNPSPNLNSPPPEPKNTKFVNNLEKDHISSLYNLVKSSVDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECR

SEQ ID NO: 56 - protein, 341 aa, synthetic construct
MIGILLLIGICVAVTVAILYSMYNKIKNSQNPNPSPNLNSPPPEPKNTKFVNNLEKDHISSLYNLVKSSVGGGGSDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGTGKPIPNPLLGLDST

SEQ ID NO: 57 - DNA, 1107 bp, synthetic construct
  source 1..1107: mol_type = other DNA
atgtcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgccacgagatatcgataatggccaacttgatattggtggagtagactttggctctagtataacgtactcttgtaatagcggatatcatttgatcggtgaatctaaatcgtattgtgaattaggatctactggatctatggtatggaatcccgaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgcggcggaggtggaagtggaggcggtggatccggtggaggaggcagcgacggaactcttttccccggagatgacgatcttgcaattccagcaactgaatttttttctacaaaggctgataaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgag

SEQ ID NO: 58 - DNA, 1107 bp, synthetic construct
  source 1..1107: mol_type = other DNA
atgtcctgtactattccgtcacgacccattaatatgaaatttaagaatagtgtggagactgatgctaatgctaattacaacataggagacactatagaatatctatgtctacctggatacagaaagcaaaaaatgggacctatatatgctaaatgtacaggtactggatggacactctttaatcaatgtattaaacggagatgcccatcgccacgagatatcgataatggccaccttgatattggtggagtagactttggctctagtataacgtactcttgtaatagcggatatcatttgatcggtgaatctaaatcgtattgtgaattaggatctactggatctatggtatggaatcccaaggcacctatttgtgaatctgttaaatgccaatcccctccatctatatccaacggaagacataacggatacgaggatttctacaccgatgggagcgttgtaacttatagttgcaatagtggatattcgttgattggtaactctggtgtcctgtgttcaggaggagaatggtccgatccacccacgtgtcagattgttaaatgtccacatcctacaatatcaaacggatacttgtctagcgggtttaaaagatcatactcatacaacgacaatgtagactttaagtgcaagtacggatataaactatctggttcctcatcatctacttgctctccaggaaatacatggaagccggaacttccaaaatgtgtacgcggcggaggtggaagtggaggcggtggatccggtggaggaggcagcgacggaactcttttccccggagatgacgatcttgcaattccagcaactgaatttttttctacaaaggctgataaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgag

SEQ ID NO: 59 - DNA, 1125 bp, synthetic construct
  source 1..1125: mol_type = other DNA
atggactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggcggaggtggaagtggaggcggtggatccggtggaggaggcagcgacggaactcttttccccggagatgacgatcttgcaattccagcaactgaatttttttctacaaaggctgataaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgag

SEQ ID NO: 60 - DNA, 5004 bp, synthetic construct
  source 1..5004: mol_type = other DNA
atgcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattataggcggaggtggaagtggaggcggtggatccggtggaggaggcagcgacggaactcttttccccggagatgacgatcttgcaattccagcaactgaatttttttctacaaaggctgataaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgag

SEQ ID NO: 61 - DNA, 1113 bp, synthetic construct
  source 1..1113: mol_type = other DNA
atgttaagctgtcctacacgtaaccagtatgtttctgtcaaatatgtgaatctaactaactattcaggcccgtatccaaacgggacaacgctacacgtgacatgccgtgaaggatatgcaaaaagaccagtacaaactgttacatgcgtcaatggtaactggactgtacctaaaaagtgtcagaaaaagaaatgttctacaccgcaagatcttttaaatggaagatatactgtaactggtaatttatattacggttcagttatcacttatacttgtaattcaggctacagcttaattggaagcacaacatcagcttgtttacttaaacgaggtggtcgtgttgactggactccacgacctccaatttgtgacattaaaaaatgtaaacctcctccacaaatagctaatgggactcacactaatgtcaaagatttctatacttatttagatacagttacgtactcatgcaatgacgaaacaaagttaactttaacaggcccttcatcgaaactttgttcagaaactggctcatgggtacctaatggagaaactaagtgtgaatttatattttgtaaactacctcaagttgcgaatgcgtacgttgaagttagaaagtcagctacgagcatgcaatatttgcatataaatgttaaatgttataaaggatttatgctatatggagaaactcctaatacgtgtaaccatggagtatggtctccagctattcctgaatgtatgaagggcggaggtggaagtggaggcggtggatccggtggaggaggcagcgacggaactcttttccccggagatgacgatcttgcaattccagcaactgaatttttttctacaaaggctgataaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgag

SEQ ID NO: 62 - DNA, 1113 bp, synthetic construct
  source 1..1113: mol_type = other DNA
atgtgccaccatcttcctcaaatgcccacactaacatctcctaaatttacaccaccagtcaaaagtggcaccaccctgcagctccgctgtaggccagggttcacaccaggcgcaggggggcggaagacagtaacctgcttaggggataatatgtggacaaccgtgactccctgtaacagaaagaggtgcccacacgttacctttccaaccaatgggggcgctaattaccattttaaagacaatgataccagtcctagctatggtaccgaggcagtgttttactgtgatcccggctacaatcttctgggagaaagtaaattattttgtgagctgcaggataataataaagtgggctggagtggggaatctcccatatgtgacataaaaaagtgtgcacctcccgaagtcaagtccccagcctatatactgagggccaaagatgtgtataacacccgagaggtgaccagatttagttgtcctataaatctgaagctatatgggccaacccacgcagtctgtgagggacccgggtggacaccatccaccagtcccttttgcctggaaattccctgtggccaacctacaattccgcatgccacaattcaatctaagccaggggctcccccaggctctagagaacttgttgtcaattgcgatccaggttacatgcccctagatgggaccaccttgacatgcgaaggcagaggtgtttggaaactgccccttcctgaatgtgttgccggcggaggtggaagtggaggcggtggatccggtggaggaggcagcgacggaactcttttccccggagatgacgatcttgcaattccagcaactgaatttttttctacaaaggctgataaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgag

SEQ ID NO: 63 - protein, 369 aa, synthetic construct
MSCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVRGGGGSGGGGSGGGGSDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 64 - protein, 369 aa, synthetic construct
MSCTIPSRPINMKFKNSVETDANANYNIGDTIEYLCLPGYRKQKMGPIYAKCTGTGWTLFNQCIKRRCPSPRDIDNGHLDIGGVDFGSSITYSCNSGYHLIGESKSYCELGSTGSMVWNPKAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVRGGGGSGGGGSGGGGSDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 65 - protein, 375 aa, synthetic construct
MDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGGGGSGGGGSGGGGSDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 66 - protein, 1668 aa, synthetic construct
MQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIGGGGSGGGGSGGGGSDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 67 - protein, 371 aa, synthetic construct
MLSCPTRNQYVSVKYVNLTNYSGPYPNGTTLHVTCREGYAKRPVQTVTCVNGNWTVPKKCQKKKCSTPQDLLNGRYTVTGNLYYGSVITYTCNSGYSLIGSTTSACLLKRGGRVDWTPRPPICDIKKCKPPPQIANGTHTNVKDFYTYLDTVTYSCNDETKLTLTGPSSKLCSETGSWVPNGETKCEFIFCKLPQVANAYVEVRKSATSMQYLHINVKCYKGFMLYGETPNTCNHGVWSPAIPECMKGGGGSGGGGSGGGGSDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 68 - protein, 371 aa, synthetic construct
MCHHLPQMPTLTSPKFTPPVKSGTTLQLRCRPGFTPGAGGRKTVTCLGDNMWTTVTPCNRKRCPHVTFPTNGGANYHFKDNDTSPSYGTEAVFYCDPGYNLLGESKLFCELQDNNKVGWSGESPICDIKKCAPPEVKSPAYILRAKDVYNTREVTRFSCPINLKLYGPTHAVCEGPGWTPSTSPFCLEIPCGQPTIPHATIQSKPGAPPGSRELVVNCDPGYMPLDGTTLTCEGRGVWKLPLPECVAGGGGSGGGGSGGGGSDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 69 - DNA, 42 bp, synthetic construct
  source 1..42: mol_type = other DNA
ggtaagcctatccctaaccctctcctcggtctcgattctacg

SEQ ID NO: 70 - protein, 14 aa, synthetic construct
GKPIPNPLLGLDST

SEQ ID NO: 71 - protein, 250 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECR

SEQ ID NO: 72 - protein, 1543 aa, Homo sapiens
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 73 - protein, 246 aa, Ateline gammaherpesvirus 3
LSCPTRNQYVSVKYVNLTNYSGPYPNGTTLHVTCREGYAKRPVQTVTCVNGNWTVPKKCQKKKCSTPQDLLNGRYTVTGNLYYGSVITYTCNSGYSLIGSTTSACLLKRGGRVDWTPRPPICDIKKCKPPPQIANGTHTNVKDFYTYLDTVTYSCNDETKLTLTGPSSKLCSETGSWVPNGETKCEFIFCKLPQVANAYVEVRKSATSMQYLHINVKCYKGFMLYGETPNTCNHGVWSPAIPECMK

SEQ ID NO: 74 - protein, 247 aa, Retroperitoneal fibromatosis-associated herpesvirus
MCHHLPQMPTLTSPKFTPPVKSGTTLQLRCRPGFTPGAGGRKTVTCLGDNMWTTVTPCNRKRCPHVTFPTNGGANYHFKDNDTSPSYGTEAVFYCDPGYNLLGESKLFCELQDNNKVGWSGESPICDIKKCAPPEVKSPAYILRAKDVYNTREVTRFSCPINLKLYGPTHAVCEGPGWTPSTSPFCLEIPCGQPTIPHATIQSKPGAPPGSRELVVNCDPGYMPLDGTTLTCEGRGVWKLPLPECVA

SEQ ID NO: 75 - protein, 262 aa, Ectromelia virus
MKVESVTFLTLLGIGCVLSCCTIPSRPINMKFKNNVGTDDTYKVGDTIEYVCLPGYRKQKMGSIYVKCTGNGWTLFNQCIKRRCPSPRDIDNGQLDIGGTDFGSSITYSCNSGYQLIGESKSYCELGSTGSMVWNPEAPICESVKCQSPPSISNGRHNGYEDFYTEGSVVTYSCNSGYSLVGNSGVLCSGGEWSDPPTCQIVKCPHPTISNGYLSSGFKRSYSYNDNVDFKCKYGYKLSGSSSSTCSPENTWQPKLPKCVRE

SEQ ID NO: 76 - protein, 243 aa, Ectromelia virus
CCTIPSRPINMKFKNNVGTDDTYKVGDTIEYVCLPGYRKQKMGSIYVKCTGNGWTLFNQCIKRRCPSPRDIDNGQLDIGGTDFGSSITYSCNSGYQLIGESKSYCELGSTGSMVWNPEAPICESVKCQSPPSISNGRHNGYEDFYTEGSVVTYSCNSGYSLVGNSGVLCSGGEWSDPPTCQIVKCPHPTISNGYLSSGFKRSYSYNDNVDFKCKYGYKLSGSSSSTCSPENTWQPKLPKCVRE

SEQ ID NO: 77 - protein, 259 aa, Cowpox virus
MKVESVTFLTLLGIGCVLSCCPIPSRPITMKFKGTVDSHYNIGDTIEYLCLPGYRKQKMGPIYAKCTGTGWTLFNQCIKRRCPSPRDIDNGQLDIGGVDFGSSITYSCNSGYQLIGESKSYCELGSTGSMVWNPEAPICESVKCQSPPSISNGRHNGYEDFYTDGSVVTYSCNSGYSLIGNSAVLCSGGEWSDPPTCQIVKCPHPTISNGYLSSGFKRSYSYNDNVDFKCKYGYKLSGSSSSTCSPGNTWKPELPKCVR

SEQ ID NO: 78 - protein, 240 aa, Cowpox virus
CCPIPSRPITMKFKGTVDSHYNIGDTIEYLCLPGYRKQKMGPIYAKCTGTGWTLFNQCIKRRCPSPRDIDNGQLDIGGVDFGSSITYSCNSGYQLIGESKSYCELGSTGSMVWNPEAPICESVKCQSPPSISNGRHNGYEDFYTDGSVVTYSCNSGYSLIGNSAVLCSGGEWSDPPTCQIVKCPHPTISNGYLSSGFKRSYSYNDNVDFKCKYGYKLSGSSSSTCSPGNTWKPELPKCVR